SEQUENCE LISTING (123 sequences)

SEQ ID NO: 1 - RNA, 23 nt, synthetic construct
  source 1..23: mol_type = other RNA
cgccatggtgatgtaccccaggg

SEQ ID NO: 2 - DNA, 20 bp, synthetic construct
  source 1..20: mol_type = other DNA
agccatgatgatgcaccaca

SEQ ID NO: 3 - DNA, 20 bp, synthetic construct
  source 1..20: mol_type = other DNA
ccccatggtgatggacagca

SEQ ID NO: 4 - DNA, 20 bp, synthetic construct
  source 1..20: mol_type = other DNA
ccccatggtgatggacagca

SEQ ID NO: 5 - DNA, 20 bp, synthetic construct
  source 1..20: mol_type = other DNA
ccccatggtgatggacagca

SEQ ID NO: 6 - DNA, 20 bp, synthetic construct
  source 1..20: mol_type = other DNA
ccccaaggtgatgttcacca

SEQ ID NO: 7 - DNA, 20 bp, synthetic construct
  source 1..20: mol_type = other DNA
cgccatgattatgtccctca

SEQ ID NO: 8 - DNA, 20 bp, synthetic construct
  source 1..20: mol_type = other DNA
caccatggagatcaacccca

SEQ ID NO: 9 - DNA, 20 bp, synthetic construct
  source 1..20: mol_type = other DNA
caccatggtgatgtacacag

SEQ ID NO: 10 - DNA, 20 bp, synthetic construct
  source 1..20: mol_type = other DNA
ccccatggtgatggatgcca

SEQ ID NO: 11 - DNA, 20 bp, synthetic construct
  source 1..20: mol_type = other DNA
agccatggtgaggtccccga

SEQ ID NO: 12 - DNA, 20 bp, synthetic construct
  source 1..20: mol_type = other DNA
ggccatgtggatgaacccca

SEQ ID NO: 13 - DNA, 20 bp, synthetic construct
  source 1..20: mol_type = other DNA
tgccatggggatgcatccca

SEQ ID NO: 14 - DNA, 20 bp, synthetic construct
  source 1..20: mol_type = other DNA
ccccatggttatctacccaa

SEQ ID NO: 15 - DNA, 20 bp, synthetic construct
  source 1..20: mol_type = other DNA
cgccttgctgatggtcccca

SEQ ID NO: 16 - DNA, 20 bp, synthetic construct
  source 1..20: mol_type = other DNA
ccccacggtgatgagcccca

SEQ ID NO: 17 - DNA, 20 bp, synthetic construct
  source 1..20: mol_type = other DNA
caccatggtggtgttctcca

SEQ ID NO: 18 - DNA, 20 bp, synthetic construct
  source 1..20: mol_type = other DNA
ctccatggtgaagagcccca

SEQ ID NO: 19 - DNA, 20 bp, synthetic construct
  source 1..20: mol_type = other DNA
ctccattgagatgtatccca

SEQ ID NO: 20 - DNA, 20 bp, synthetic construct
  source 1..20: mol_type = other DNA
ctccaaggtgatgtcaccca

SEQ ID NO: 21 - DNA, 20 bp, synthetic construct
  source 1..20: mol_type = other DNA
ctccatgatgctgtccccca

SEQ ID NO: 22 - RNA, 23 nt, synthetic construct
  source 1..23: mol_type = other RNA
ctgcccacactgagcacggacgg

SEQ ID NO: 23 - DNA, 20 bp, synthetic construct
  source 1..20: mol_type = other DNA
atgcgcacactgagcaggga

SEQ ID NO: 24 - DNA, 20 bp, synthetic construct
  source 1..20: mol_type = other DNA
ctgtcgacactgagcaagga

SEQ ID NO: 25 - DNA, 20 bp, synthetic construct
  source 1..20: mol_type = other DNA
ctgcccacactgacaaccga

SEQ ID NO: 26 - DNA, 20 bp, synthetic construct
  source 1..20: mol_type = other DNA
ctgcacactctgagaacgga

SEQ ID NO: 27 - DNA, 20 bp, synthetic construct
  source 1..20: mol_type = other DNA
atgcccacactgaccacggc

SEQ ID NO: 28 - DNA, 20 bp, synthetic construct
  source 1..20: mol_type = other DNA
gagaccacactgagcacgga

SEQ ID NO: 29 - DNA, 20 bp, synthetic construct
  source 1..20: mol_type = other DNA
ctgccaacactgagtacggt

SEQ ID NO: 30 - DNA, 20 bp, synthetic construct
  source 1..20: mol_type = other DNA
cagcccacactgagccagga

SEQ ID NO: 31 - DNA, 20 bp, synthetic construct
  source 1..20: mol_type = other DNA
ctccccacactgagtactga

SEQ ID NO: 32 - DNA, 20 bp, synthetic construct
  source 1..20: mol_type = other DNA
ctgcccacccagagcacaga

SEQ ID NO: 33 - DNA, 20 bp, synthetic construct
  source 1..20: mol_type = other DNA
ctgcacactctgagaacgga

SEQ ID NO: 34 - DNA, 20 bp, synthetic construct
  source 1..20: mol_type = other DNA
ctgcccacacacagcaggga

SEQ ID NO: 35 - DNA, 20 bp, synthetic construct
  source 1..20: mol_type = other DNA
ctgcccaccttgaacacaga

SEQ ID NO: 36 - DNA, 20 bp, synthetic construct
  source 1..20: mol_type = other DNA
cttcccacactgaagaggga

SEQ ID NO: 37 - DNA, 20 bp, synthetic construct
  source 1..20: mol_type = other DNA
ctgcacatactgagcatgta

SEQ ID NO: 38 - DNA, 20 bp, synthetic construct
  source 1..20: mol_type = other DNA
ctgcccccacagggcatgga

SEQ ID NO: 39 - DNA, 20 bp, synthetic construct
  source 1..20: mol_type = other DNA
ctgaccacactcaggatgga

SEQ ID NO: 40 - DNA, 20 bp, synthetic construct
  source 1..20: mol_type = other DNA
gtggccacactgtgcacaga

SEQ ID NO: 41 - RNA, 23 nt, synthetic construct
  source 1..23: mol_type = other RNA
  modified_base 21: mod_base = OTHER; note = thymine
cagtccgtccgtgctcagtgtgg

SEQ ID NO: 42 - DNA, 20 bp, synthetic construct
  source 1..20: mol_type = other DNA
cagtccttcctttctcagtg

SEQ ID NO: 43 - DNA, 20 bp, synthetic construct
  source 1..20: mol_type = other DNA
aagtccgtgtgtgctcagtg

SEQ ID NO: 44 - DNA, 20 bp, synthetic construct
  source 1..20: mol_type = other DNA
cgccccgtccgtgctcagtg

SEQ ID NO: 45 - DNA, 20 bp, synthetic construct
  source 1..20: mol_type = other DNA
cagtccgtcagagctctgtg

SEQ ID NO: 46 - DNA, 20 bp, synthetic construct
  source 1..20: mol_type = other DNA
cagtccgtccatccacaggg

SEQ ID NO: 47 - DNA, 20 bp, synthetic construct
  source 1..20: mol_type = other DNA
cagtcccacagtgcacagtg

SEQ ID NO: 48 - DNA, 20 bp, synthetic construct
  source 1..20: mol_type = other DNA
ctgtcagtccttgatcagtg

SEQ ID NO: 49 - DNA, 20 bp, synthetic construct
  source 1..20: mol_type = other DNA
cagtctttccttgctcagag

SEQ ID NO: 50 - DNA, 20 bp, synthetic construct
  source 1..20: mol_type = other DNA
gagtccgtccgtgatcacag

SEQ ID NO: 51 - DNA, 20 bp, synthetic construct
  source 1..20: mol_type = other DNA
catgcagtccgtgctcagta

SEQ ID NO: 52 - DNA, 20 bp, synthetic construct
  source 1..20: mol_type = other DNA
aagcccatctgtgctcagtg

SEQ ID NO: 53 - DNA, 20 bp, synthetic construct
  source 1..20: mol_type = other DNA
cactccgtcctttgtcagtg

SEQ ID NO: 54 - DNA, 20 bp, synthetic construct
  source 1..20: mol_type = other DNA
cagtcagtccctggacagtg

SEQ ID NO: 55 - DNA, 20 bp, synthetic construct
  source 1..20: mol_type = other DNA
cagtcagtctctggtcagtg

SEQ ID NO: 56 - DNA, 20 bp, synthetic construct
  source 1..20: mol_type = other DNA
cagtgcgtctcagctcagtg

SEQ ID NO: 57 - DNA, 20 bp, synthetic construct
  source 1..20: mol_type = other DNA
ccgtccgtcccagctcagcg

SEQ ID NO: 58 - DNA, 20 bp, synthetic construct
  source 1..20: mol_type = other DNA
cattccttctgtgctcagtt

SEQ ID NO: 59 - DNA, 20 bp, synthetic construct
  source 1..20: mol_type = other DNA
cagtcagtcccagcccagtg

SEQ ID NO: 60 - DNA, 20 bp, synthetic construct
  source 1..20: mol_type = other DNA
cagtccctcggccctcagtg

SEQ ID NO: 61 - RNA, 23 nt, synthetic construct
  source 1..23: mol_type = other RNA
tagggaacagcaatattgcgggg

SEQ ID NO: 62 - DNA, 20 bp, synthetic construct
  source 1..20: mol_type = other DNA
tagggatcagcactattggg

SEQ ID NO: 63 - DNA, 20 bp, synthetic construct
  source 1..20: mol_type = other DNA
tagggaacagcaatattata

SEQ ID NO: 64 - DNA, 20 bp, synthetic construct
  source 1..20: mol_type = other DNA
tagtgaacagcaattttgtg

SEQ ID NO: 65 - DNA, 20 bp, synthetic construct
  source 1..20: mol_type = other DNA
tagggaacagcactattagg

SEQ ID NO: 66 - DNA, 20 bp, synthetic construct
  source 1..20: mol_type = other DNA
taggaaacagtaatattgct

SEQ ID NO: 67 - DNA, 20 bp, synthetic construct
  source 1..20: mol_type = other DNA
tagagaacatcagtattgcg

SEQ ID NO: 68 - DNA, 20 bp, synthetic construct
  source 1..20: mol_type = other DNA
tagggaacagcactattagg

SEQ ID NO: 69 - DNA, 20 bp, synthetic construct
  source 1..20: mol_type = other DNA
tagggaacaggaatcttggg

SEQ ID NO: 70 - DNA, 20 bp, synthetic construct
  source 1..20: mol_type = other DNA
tagggaatagcactattggg

SEQ ID NO: 71 - DNA, 20 bp, synthetic construct
  source 1..20: mol_type = other DNA
cagggaacagcactattgca

SEQ ID NO: 72 - DNA, 20 bp, synthetic construct
  source 1..20: mol_type = other DNA
tagggaacggcactattggg

SEQ ID NO: 73 - DNA, 20 bp, synthetic construct
  source 1..20: mol_type = other DNA
taaggaaaagcaatattgag

SEQ ID NO: 74 - DNA, 20 bp, synthetic construct
  source 1..20: mol_type = other DNA
tagggagcggcactattggg

SEQ ID NO: 75 - DNA, 20 bp, synthetic construct
  source 1..20: mol_type = other DNA
tagggaacggcactattagg

SEQ ID NO: 76 - DNA, 20 bp, synthetic construct
  source 1..20: mol_type = other DNA
tggggaacagcactattaag

SEQ ID NO: 77 - DNA, 20 bp, synthetic construct
  source 1..20: mol_type = other DNA
tagggaatagcactattagg

SEQ ID NO: 78 - DNA, 20 bp, synthetic construct
  source 1..20: mol_type = other DNA
tagggagtagcactattggg

SEQ ID NO: 79 - DNA, 20 bp, synthetic construct
  source 1..20: mol_type = other DNA
cagggagcagcactattggg

SEQ ID NO: 80 - DNA, 20 bp, synthetic construct
  source 1..20: mol_type = other DNA
tagggaatagcactattaag

SEQ ID NO: 81 - DNA, 20 bp, synthetic construct
  source 1..20: mol_type = other DNA
tagggaatggcagtattgag

SEQ ID NO: 82 - RNA, 23 nt, synthetic construct
  source 1..23: mol_type = other RNA
  modified_base 21: mod_base = OTHER; note = thymine
gtgcaaggcgctgtgacttgtgg

SEQ ID NO: 83 - DNA, 20 bp, synthetic construct
  source 1..20: mol_type = other DNA
gtgccaggctctgtgacctg

SEQ ID NO: 84 - DNA, 20 bp, synthetic construct
  source 1..20: mol_type = other DNA
gtggaaggcgctgtgggttg

SEQ ID NO: 85 - DNA, 20 bp, synthetic construct
  source 1..20: mol_type = other DNA
gtgcctggctctgtgacttg

SEQ ID NO: 86 - DNA, 20 bp, synthetic construct
  source 1..20: mol_type = other DNA
gtggaaggcacagtgacttg

SEQ ID NO: 87 - DNA, 20 bp, synthetic construct
  source 1..20: mol_type = other DNA
gtgctaggggctgtgactcg

SEQ ID NO: 88 - DNA, 20 bp, synthetic construct
  source 1..20: mol_type = other DNA
tggcaaggcgctgtgacttc

SEQ ID NO: 89 - DNA, 20 bp, synthetic construct
  source 1..20: mol_type = other DNA
ttgcatggcgctgagacttg

SEQ ID NO: 90 - DNA, 20 bp, synthetic construct
  source 1..20: mol_type = other DNA
gggcaagtcgctgttacttg

SEQ ID NO: 91 - DNA, 20 bp, synthetic construct
  source 1..20: mol_type = other DNA
gtgccaggctctgtgacgtg

SEQ ID NO: 92 - DNA, 20 bp, synthetic construct
  source 1..20: mol_type = other DNA
gtgcaggccgctgtggcttg

SEQ ID NO: 93 - DNA, 20 bp, synthetic construct
  source 1..20: mol_type = other DNA
gaggaaggcgctgtgactgg

SEQ ID NO: 94 - DNA, 20 bp, synthetic construct
  source 1..20: mol_type = other DNA
gtgccaagtgctgtgacctg

SEQ ID NO: 95 - DNA, 20 bp, synthetic construct
  source 1..20: mol_type = other DNA
atgccaggcgctgtgaatta

SEQ ID NO: 96 - DNA, 20 bp, synthetic construct
  source 1..20: mol_type = other DNA
gtgcagggagcagtggcttg

SEQ ID NO: 97 - DNA, 20 bp, synthetic construct
  source 1..20: mol_type = other DNA
gtgtcaggctctgtgacttt

SEQ ID NO: 98 - DNA, 20 bp, synthetic construct
  source 1..20: mol_type = other DNA
gtgcaaggcgctgggggtag

SEQ ID NO: 99 - DNA, 20 bp, synthetic construct
  source 1..20: mol_type = other DNA
gagcaagctgctgagacttg

SEQ ID NO: 100 - DNA, 20 bp, synthetic construct
  source 1..20: mol_type = other DNA
gtggaaagcactgggacttg

SEQ ID NO: 101 - DNA, 20 bp, synthetic construct
  source 1..20: mol_type = other DNA
gtgccaggcactgtgcctgg

SEQ ID NO: 102 - DNA, 20 bp, synthetic construct
  source 1..20: mol_type = other DNA
gtgcaaggtgctgggactat

SEQ ID NO: 103 - RNA, 23 nt, synthetic construct
  source 1..23: mol_type = other RNA
tttgccccacacatatgccatgg

SEQ ID NO: 104 - DNA, 20 bp, synthetic construct
  source 1..20: mol_type = other DNA
tttaccccacgcatatgcct

SEQ ID NO: 105 - DNA, 20 bp, synthetic construct
  source 1..20: mol_type = other DNA
tctgccccacacacatccca

SEQ ID NO: 106 - DNA, 20 bp, synthetic construct
  source 1..20: mol_type = other DNA
tttgccacacagatatgcct

SEQ ID NO: 107 - DNA, 20 bp, synthetic construct
  source 1..20: mol_type = other DNA
tttccgccacacagatgcca

SEQ ID NO: 108 - DNA, 20 bp, synthetic construct
  source 1..20: mol_type = other DNA
ttttgcccccacatatgcca

SEQ ID NO: 109 - DNA, 20 bp, synthetic construct
  source 1..20: mol_type = other DNA
tttgccacccacatatacca

SEQ ID NO: 110 - DNA, 20 bp, synthetic construct
  source 1..20: mol_type = other DNA
attgtcccacacataagcca

SEQ ID NO: 111 - DNA, 20 bp, synthetic construct
  source 1..20: mol_type = other DNA
cttcccccactcatatgcca

SEQ ID NO: 112 - DNA, 20 bp, synthetic construct
  source 1..20: mol_type = other DNA
tttgacacacacagatgcca

SEQ ID NO: 113 - DNA, 20 bp, synthetic construct
  source 1..20: mol_type = other DNA
tttgcactacacacatgcca

SEQ ID NO: 114 - DNA, 20 bp, synthetic construct
  source 1..20: mol_type = other DNA
gttgccccacaccaatgcta

SEQ ID NO: 115 - DNA, 20 bp, synthetic construct
  source 1..20: mol_type = other DNA
tttcctccatacatatacca

SEQ ID NO: 116 - DNA, 20 bp, synthetic construct
  source 1..20: mol_type = other DNA
ttttccacacacatgtacca

SEQ ID NO: 117 - DNA, 20 bp, synthetic construct
  source 1..20: mol_type = other DNA
ttttccccacaaatatccct

SEQ ID NO: 118 - DNA, 20 bp, synthetic construct
  source 1..20: mol_type = other DNA
attgccccacagatgtccca

SEQ ID NO: 119 - DNA, 20 bp, synthetic construct
  source 1..20: mol_type = other DNA
tttgccccacagagatgctt

SEQ ID NO: 120 - DNA, 20 bp, synthetic construct
  source 1..20: mol_type = other DNA
tttgacccacccacatgtca

SEQ ID NO: 121 - DNA, 20 bp, synthetic construct
  source 1..20: mol_type = other DNA
ctggccccagacatatgaca

SEQ ID NO: 122 - DNA, 20 bp, synthetic construct
  source 1..20: mol_type = other DNA
tttgccacagtcataggcca

SEQ ID NO: 123 - DNA, 20 bp, synthetic construct
  source 1..20: mol_type = other DNA
tttcccccacccatatccta

(19) United States
(12) Patent Application Publication
(10) Pub. No.: US 2024/0350669 A1
(43) Pub. Date: Oct. 24, 2024

(54) INHIBITION OF KYNURENINE SYNTHESIS AND/OR SIGNALING TO TREAT LEUKEMIA AND MYELODYSPLASIA

(71) Applicant: THE TRUSTEES OF COLUMBIA UNIVERSITY IN THE CITY OF NEW YORK, New York, NY (US)

(72) Inventors: Stavroula KOUSTENI, New York, NY (US); Marta GALÁN-DÍEZ, New York, NY (US)

(73) Assignee: THE TRUSTEES OF COLUMBIA UNIVERSITY IN THE CITY OF NEW YORK, New York, NY (US)

(21) Appl. No.: 18/762,799

(22) Filed: Jul. 3, 2024

Related U.S. Application Data
(63) Continuation of application No. PCT/US23/60230, filed on Jan. 6, 2023.
(60) Provisional application No. 63/297,390, filed on Jan. 7, 2022.

(51) Int. Cl. A61K 48/00; A61K 31/4245; A61P 35/02; C07K 16/18; C12N 9/22; C12N 15/11; C12N 15/113

(52) U.S. Cl. CPC A61K 48/005; A61K 31/4245; A61P 35/02; C07K 16/18; C12N 9/22; C12N 15/111; C12N 15/1138; C12N 2310/14; C12N 2310/20; C12Y 113/11052

(57) ABSTRACT

Methods and compositions for treating leukemia involving administering a therapeutically effective amount of an inhibitor of indoleamine 2,3 dioxygenase (IDO1). The leukemia may be is acute myeloid leukemia or acute lymphoid leukemia. The inhibitor can be a small molecule such as indiximod, epacadostat, BMS-986205, navoximod, PF-0684003, KHK2455 or LY3381916 or epacadostat. The inhibitor can be used alone or in conjunctions with other chemotherapeutic agents. IDO1 can also be inhibited using a CRISP-CAS system. The inhibitor can be administered orally, intravenously, intramuscularly, topically, arterially, or subcutaneously.

### CROSS REFERENCE TO RELATED APPLICATIONS

The present application is a Continuation of PCT/US23/060230, filed Jan. 6, 2023, which claims priority to U.S. provisional patent application No. 63/297,390, filed Jan. 7, 2022, both of which are hereby incorporated by reference in their entireties.

### GOVERNMENT SUPPORT

This invention was made with government support under AR054447 and HL130937 awarded by the National Institutes of Health. The government has certain rights in the invention.

### SEQUENCE LISTING

The application contains a sequence listing which has been submitted electronically in XML format and is hereby incorporated by reference in its entirety. Said XML copy, created on Jan. 6, 2023, is named 01001_009983-WO0_ST26.xml and is 110,394 bytes in size.

### FIELD OF THE DISCLOSURE

The present disclosure relates to the treatment of leukemia and myelodysplasia via the inhibition of kynurenine synthesis and/or signaling.

### BACKGROUND OF THE DISCLOSURE

Hematological malignancies have long been thought to be exclusively driven by genetic or epigenetic mutations within hematopoietic cells. Besides these classical mechanisms, demonstrated in animal models and human cells, there is increasing evidence that the bone marrow (BM) microenvironment or niche plays a role in the pathogenesis, maintenance and resistance to treatment of malignant clones. Accordingly, the niche can enable immune evasion and activation of survival and differentiation pathways favoring malignant-cell maintenance, defense against oxidative stress and protection from chemotherapy.

As mentioned above, recent studies indicate that the tumor microenvironment plays an important role in disease development. For example, osteoblasts are cells important for the formation of new bone and have been found to exert a tumor-suppressive role in AML, elucidating a potential mechanism for therapeutic targeting and development.

Acute myeloid leukemia (AML), a heterogeneous clonal hematopoietic neoplasm and one of the most common hematological malignancies of the elderly, remains recalcitrant to targeted therapies due to the emergence of pre-existent or de novo therapy-resistant leukemic clones. Against this backdrop, cell non-autonomous contributions of the niche to disease development, propagation and maintenance may hold promise for the development of new treatment approaches that focus on the niche which sustains AML. Particularly among niches, alterations in the osteoblastic compartment can lead to myelodysplastic syndrome (MDS) and AML in mice, and are associated with myeloproliferative neoplasms, MDS and AML in patients. In addition, osteoblasts can exert a tumor-suppressor role in myeloid disorders or can be remodeled by dysplastic cells to reinforce leukemia. Osteoblast numbers are decreased in MDS and AML patients and their ablation increases leukemia burden, whereas maintaining the osteoblastic pool, reduces tumor burden and prolongs survival. However, the mechanisms that mediate the leukemia cell-osteoblast communication, the molecular events that affect leukemia outcome, and the question whether this crosstalk could be harnessed for a therapeutic purpose remain largely unexplored.

AML progression requires the presence of serotonin receptor-1b (HTR1B) in osteoblasts and is driven by AML-secreted kynurenine, which acts as an oncometabolite and HTR1B ligand. AML cells utilize kynurenine to induce a pro-inflammatory state in osteoblasts which, through the acute-phase protein serum amyloid A (SAA), acts in a positive feedback-loop on leukemia cells by increasing expression of indoleamine 2,3-dioxygenase (IDO1), a rate-limiting enzyme for kynurenine synthesis, thereby enabling AML progression.

Rather than target the tumor cells directly, there is a need for therapies which target other causes of AML, such as the tumor microenvironment (Krevvata M, et al., Inhibition of leukemia cell engraftment and disease progression in mice by osteoblasts. Blood. 2014 October; 124 (18): pp. 2834-46). Specifically, osteoblasts can be targeted to inhibit leukemia engraftment and disease progression (Krevvata M, et al., Inhibition of leukemia cell engraftment and disease progression in mice by osteoblasts. Blood. 2014 October; 124 (18): pp. 2834-46).

Consequently, there is a need to develop inhibitors of kynurenine synthesis such as IDO1 to treat leukemias as well as other myelodysplastic syndromes.

### SUMMARY OF THE DISCLOSURE

The invention provides for a methods and compositions for treating leukemia comprising administering a therapeutically effective amount of an inhibitor of indoleamine 2,3 dioxygenase (IDO1) to a mammal in need thereof. In some embodiments, the mammal is a human. The leukemia may be is acute myeloid leukemia or acute lymphoid leukemia. The inhibitor comprises indiximod, epacadostat, BMS-986205, navoximod, PF-0684003, KHK2455 or LY3381916 or combinations thereof or epacadostat. The inhibitor can be administered orally, intravenously, intramuscularly, topically, arterially, or subcutaneously.

The invention also provides for methods and compositions of inhibiting indolcamine 2,3 dioxygenase expression comprising introducing into a eukaryotic cell an engineered, non-naturally occurring Clustered Regularly Interspaced Short Palindromic Repeats (CRISPR)-CRISPR associated (Cas) (CRISPR-Cas) system comprising one or more vectors comprising a) a first regulatory element operable in a eukaryotic cell operably linked to at least one nucleotide sequence encoding a CRISPR-Cas system guide RNA that hybridizes with sequences encoding exons 3 or 4 of indoleamine 2,3 dioxygenase, and b) a second regulatory element operable in a eukaryotic cell operably linked to a nucleotide sequence encoding a Cas9 protein, wherein components (a) and (b) are located on same or different vectors of the system, whereby the guide RNA targets sequences encoding exons 3 or 4 of indoleamine 2,3 dioxygenase and the Cas9 protein cleaves the DNA molecule, whereby expression of indoleamine 2,3 dioxygenase protein is reduced; and, wherein the Cas9 protein and the guide RNA do not naturally occur together.

The invention provides for methods and compositions for treating leukemia in a subject, comprising administering a therapeutically effective amount of a modulator of indoleamine 2,3 dioxygenase to a subject. The modulator can bind to the enzyme catalytic site of indoleamine 2,3 dioxygenase. The modulator can be a small molecule, a polynucleotide, or an antibody or antigen-binding portion thereof. In other embodiments, the modulator is a nucleic acid chosen from the group consisting of a single-stranded DNA (ssDNA), a double-stranded DNA (dsDNA), a donor/template DNA, s cDNA, a DNA encoding one or more RNAs, a sgRNA, a guide RNA (gRNA), a prime editing guide RNA (pegRNA), a microRNA (miRNA) inhibitor, a miRNA mimic, a small interfering RNA (siRNA), small synthetic RNA, a synthetic RNA, an antisense oligonucleotide, a short hairpin RNA (shRNA), a double-stranded RNA (dsRNA), an antisense RNA, a ribozyme, and combinations thereof. Preferably, the modulate can be a polynucleotide such as a small interfering RNA (siRNA) or an antisense molecule. The modulator can be administered orally, intravenously, intramuscularly, topically, arterially, or subcutaneously.

The invention also provides for compositions and methods for treating myelodysplastic syndrome comprising administering a therapeutically effective amount of an inhibitor of indoleamine 2,3 dioxygenase to a mammal in need thereof. The mammal can be a human. In one embodiment, the inhibitor comprises indiximod, epacadostat, BMS-986205, navoximod, PF-0684003, KHK2455 or LY3381916 or combinations thereof. In another embodiment, the inhibitor comprises epacadostat. Other inhibitors such as siRNA or a CRISPR/Cas system can be used as an inhibitor. The inhibitor can be administered orally, intravenously, intramuscularly, topically, arterially, or subcutaneously.

The invention provides for methods and compositions for of treating leukemia comprising administering a therapeutically effective amount of an inhibitor of serum amyloid A1 (SAA1) to a mammal in need thereof. In some embodiments, the mammal is a human. In some embodiments, the leukemia is acute myeloid leukemia or acute lymphoid leukemia. In some embodiments, the inhibitor comprises ant an anti-SAA1 antibody or antigen-binding portion or combinations thereof. In some embodiments, the anti-SAA1 antibody is administered orally, intravenously, intramuscularly, topically, arterially, or subcutaneously.

### BRIEF DESCRIPTION OF THE DRAWINGS

FIG. 1A shows the survival curve of wild-type (WT) mice treated with vehicle (n=4) or parathyroid hormone (PTH, n=7) and injected with MLL/AF9 AML cells. All survival curves shown are Kaplan-Meier curves with the p-value of log rank (Mantel-Cox) test between the indicated groups. All data are represented as mean±SEM, statistical analysis done with unpaired t-test.

FIG. 1B shows the Survival curves of WT MLL/AF9-injected mice, their spleen weights and representative epifluorescence images (radiance p/sec/cm2/sr) of leukemia progression 14 days after MLL/AF9 injection in Htr1b−/−(n=29) and Htr1b+/+ littermates (n=13). All survival curves shown are Kaplan-Meier curves with the p-value of log rank (Mantel-Cox) test between the indicated groups. All data are represented as mean±SEM, statistical analysis done with unpaired t-test.

FIG. 1C shows the survival curves of WT MLL/AF9-injected mice, their spleen weights and representative epifluorescence images (radiance p/sec/cm2/sr) of leukemia progression 14 days after MLL/AF9 injection in Htr1b^{fl/fl}; LepR-Cre: Htr1bLep-R^{-/-} (n=8) and Htr1b^{+/+} littermates (n=6). All survival curves shown are Kaplan-Meier curves with the p-value of log rank (Mantel-Cox) test between the indicated groups. All data are represented as mean±SEM, statistical analysis done with unpaired t-test.

FIG. 1D shows the survival curves of WT MLL/AF9-injected mice, their spleen weights and representative epifluorescence images (radiance p/sec/cm2/sr) of leukemia progression 14 days after MLL/AF9 injection in Htr1bfl/fl; Col1a1-Cre: Htr1b c-osb−/− (n=11) and Htr1b c-osb+/+ littermates (n=12)—the 4 Htr1b c-osb−/− mice that developed leukemia are; OCN-Cre: Htr1b littermates (n=10). Arrow indicates the systematic genetic interrogation approach followed. All survival curves shown are Kaplan-Meier curves with the p-value of log rank (Mantel-Cox) test between the indicated groups. All data are represented as mean±SEM, statistical analysis done with unpaired t-test.

FIG. 1E shows the survival curves of WT MLL/AF9-injected mice, their spleen weights and representative epifluorescence images (radiance p/sec/cm2/sr) of leukemia progression 14 days after MLL/AF9 injection in Htr1bfl/fl d-osb−/− (n=5) and Htr1b d-osb+/+ fl/flOsx−/− Osx+/+ Osx+/+ (DOX, n=6), Htr1b Osx−/− (no-DOX; n=9). All survival curves shown are Kaplan-Meier curves with the p-value of log rank (Mantel-Cox) test between the indicated groups. All data are represented as mean±SEM, statistical analysis done with unpaired t-test.

FIG. 1F shows Survival curves of WT MLL/AF9-injected mice, their spleen weights and representative epifluorescence images (radiance p/sec/cm2/sr) of leukemia progression 14 days after MLL/AF9 injection in survival curve of Htr1b; Osx-Cre: Htr1b (doxycycline-DOX-removed 24 h after MLL/AF9 injection; n=9) and Htr1b (kept on DOX, n=6). All survival curves shown are Kaplan-Meier curves with the p-value of log rank (Mantel-Cox) test between the indicated groups. All data are represented as mean±SEM, statistical analysis done with unpaired t-test.

FIG. 1G shows survival curves of WT MLL/AF9-injected mice, their spleen weights and representative epifluorescence images (radiance p/sec/cm2/sr) of leukemia progression 14 days after MLL/AF9 injection in Leukemia burden quantification (total flux, photons/sec) at day 12 after MLL/AF9 injection, Htr1b represented with red stars in the histogram of spleen weight and excluded from the statistical analysis.

FIG. 1H shows survival curves of WT MLL/AF9-injected mice, their spleen weights and representative epifluorescence images (radiance p/sec/cm2/sr) of leukemia progression 14 days after MLL/AF9 injection in survival curve of WT mice injected with MLL/AF9 cells and treated with either vehicle (n=10) or the HTR1B antagonist SB224289 (SB9) (n=10). All survival curves shown are Kaplan-Meier curves with the p-value of log rank (Mantel-Cox) test between the indicated groups. All data are represented as mean±SEM, statistical analysis done with unpaired t-test.

FIG. 2A shows the volcano plots for metabolites with coefficient of variation (CV) <30% comparing OCI-AML3 cells untreated (AML) and human osteoblasts (hOsb). Non-linear regression fitting was used to fit the isotherms. All data are expressed as mean±SEM.

FIG. 2B shows the volcano plots for metabolites with coefficient of variation (CV) <30% comparing OCI-AML3 cells untreated (AML) A versus co-cultures (24 h)—arrows point to kynurenine. Non-linear regression fitting was used to fit the isotherms. All data are expressed as mean±SEM.

FIG. 2C shows the Trp catabolism scheme.

FIG. 2D shows the relative abundance of tryptophan (Trp) and its catabolic metabolites: kynurenine (Kyn), serotonin (5-HT) and 5-hydroxytryptophan (5-HTP) in the indicated supernatants at 24 h (n=6); two-way ANOVA.

FIG. 2E is a heat-map of the first 30 metabolites with CV<15% and histograms of fold-change of AML vs. hOsb (scattered dots) or AML vs. co-culture (Y). Non-linear regression fitting was used to fit the isotherms. All data are expressed as mean±SEM.

FIG. 2F shows Violin plots of Kyn/Trp ratio levels in serum circulating levels of control—(n=19) vs. MLL/AF9-injected (n=28) mice; unpaired-t test. Non-linear regression fitting was used to fit the isotherms. All data are expressed as mean±SEM.

FIG. 2G shows Violin plots of Kyn/Trp ratio levels in bone marrow (BM) plasma from healthy (n=27), MDS (n=30) and AML (n=24) patients; one-way ANOVA. Non-linear regression fitting was used to fit the isotherms. All data are expressed as mean±SEM.

FIG. 2H shows Kyn/Trp levels in paired BM plasma samples at MDS stage and its corresponding transformed-AML stage (n=6); paired t-test. Non-linear regression fitting was used to fit the isotherms. All data are expressed as mean±SEM.

FIG. 2I shows RNAseq analysis of BM mononuclear cells (BM-MNCs) from MDS (n=30) and AML (n=30) patients (transcript-per-million-TPM-) for TPH1 and IDO1; two-way ANOVA. Non-linear regression fitting was used to fit the isotherms. All data are expressed as mean±SEM.

FIG. 2J shows IDO1/TPH1 mRNA ratio in BM-MNCs from healthy (n=32), MDS (n=10) and AML (n=20) patients; one-way ANOVA. Non-linear regression fitting was used to fit the isotherms. All data are expressed as mean±SEM.

FIG. 2K shows concentration dependence of the Kyn-mediated competition of [350 of 54.1 μM and 24.4 μM respectively (see Table 1 for details). Non-linear regression fitting was used to fit the isotherms. All data are expressed as mean±SEM.

FIG. 2L shows Gi/o-mediated CAMP inhibition assays (n=14). Non-linear regression fitting was used to fit the isotherms. All data are expressed as mean±SEM.

FIG. 2M shows Binding of [3H]-5-HT (25 nM, 41.3 Ci/mmol) or [3H]-Kyn (50 μM, 0.125 Ci/mmol) was measured with Htr1b-overexpressing-HEK293T membranes in the presence of increasing concentrations of SB9 (n=4). Non-linear regression fitting was used to fit the isotherms. All data are expressed as mean±SEM.

FIG. 3A shows representative epifluorescence images of leukemia progression in WT mice injected with MLL/AF9-CRISPR/Cas9-edited cells (sgRNAs: #146, #196 and #203) (Ctrl: no leukemia). All data are expressed as mean±SEM. Statistical analysis done with unpaired t-test unless otherwise stated.

FIG. 3B shows the survival curve of mice injected with the indicated sgRNAs MLL/AF9-edited or Cas9-only-MLL/AF9 control cells (n=3 all groups). All data are expressed as mean±SEM. Statistical analysis done with unpaired t-test unless otherwise stated.

FIG. 3C shows representative epifluorescence images of leukemia progression in WT mice injected with MLL/AF9-CRISPR/Cas9-edited cells (sgRNAs: #610) and Ido1 mRNA levels of MLL/AF9-sgRNA #610-edited cells before injection (n=4); unpaired t-test. All data are expressed as mean±SEM. Statistical analysis done with unpaired t-test unless otherwise stated.

FIG. 3D shows survival curve of WT mice injected with MLL/AF9-(green; n=5). All data are expressed as mean±SEM. Statistical analysis done with unpaired t-test unless otherwise stated.

FIG. 3E shows IDO1 mRNA levels in OCI-AML3 cells nucleofected with Cas9 and sgRN #610 used in transplant experiment. All data are expressed as mean±SEM. Statistical analysis done with unpaired t-test unless otherwise stated.

FIG. 3F shows IDO1 mRNA levels in OCI-AML3 cells exposed to IFN-γ (overnight, 50 ng/ml, n=3); two-way ANOVA. All data are expressed as mean±SEM. Statistical analysis done with unpaired t-test unless otherwise stated.

FIG. 3G is outline of transplantation assay with OCI-AML3 CRISPR/Cas9-IDO1-targeted cells in NSG mice. All data are expressed as mean±SEM. Statistical analysis done with unpaired t-test unless otherwise stated.

FIG. 3H shows AML burden in bone marrow, spleen, and spleen weight (mg)—referred to total body weight (g)—of NSG mice 3 weeks after injection of OCI-AML3 cells (n=8 Cas9; n=10 #126+170). All data are expressed as mean±SEM. Statistical analysis done with unpaired t-test unless otherwise stated.

FIG. 3I shows Proliferation of OCI-AML3 cells upon 72 h of co-culture with primary human osteoblasts (n=7). Survival curves are Kaplan-Meier with p-value of log rank (Mantel-Cox) test between the indicated groups.

FIG. 4A is the schematic of RNAseq analysis strategy (left) and box plots (right) of the main secreted molecules significantly upregulated in primary human osteoblasts co-cultured 24 h with the THP-1 AML cell line (n=2); Wald test, two-sided. All data expressed as mean±SEM. Statistical analysis was done with one-way ANOVA unless otherwise stated.

FIG. 4B shows Box plots for IDO1 and TPH1 from RNAseq analysis of THP-1 cells exposed 24 h to primary human osteoblasts (n=2); Wald test, two-sided. All data expressed as mean±SEM. Statistical analysis was done with one-way ANOVA unless otherwise stated.

FIG. 4C shows IDO1 mRNA levels in OCI-AML3 cells exposed o/n to the indicated molecules (UT and SAA1 n=15; IL-1α, -1β, -6, CXCL-1 and -8 n=6; IL-33, -34, and Htr1bCXCL-3, -5, CCL-2 and -20 n=3). All data expressed as mean±SEM. Statistical analysis was done with one-way ANOVA unless otherwise stated.

FIG. 4D shows Ido1 mRNA levels in WEHI-3B cells exposed o/n to recombinant mouse SAA3 or recombinant human SAA1 (n=8). All data expressed as mean±SEM. Statistical analysis was done with one-way ANOVA unless otherwise stated.

FIG. 4E shows Saa3 mRNA relative level in primary differentiated mouse calvaria from Htr1b−/−+/+ littermates, exposed for 24 h to 5-HT (25 nM, n=7-8), Kyn (25 nM, n=5) or the WEHI-3B cell line (n=10-12); two-way ANOVA. All data expressed as mean±SEM. Statistical analysis was done with one-way ANOVA unless otherwise stated.

FIG. 4F shows Violin plots of SAA3 peripheral blood (PB) serum levels in control (n=20) and MLL/AF9-injected mice (n=20); unpaired t-test. All data expressed as mean±SEM. Statistical analysis was done with one-way ANOVA unless otherwise stated.

FIG. 4G shows Violin plots of SAA1 BM plasma levels in healthy (n=30), MDS (n=35) and AML (n=23) patients. All data expressed as mean±SEM. Statistical analysis was done with one-way ANOVA unless otherwise stated.

FIG. 4H shows SAA1 BM plasma levels in paired samples from patients (MDS and corresponding AML-transformed stage) (n=6 paired-samples); paired t-test. All data expressed as mean±SEM. Statistical analysis was done with one-way ANOVA unless otherwise stated.

FIG. 4I shows multiple variable data plot of BM plasma levels for SAA1 and Kyn/Trp ratio along healthy, MDS or AML samples; Pearson correlation values are shown for Kyn/Trp ratio and SAA1 BM plasma levels. All data expressed as mean±SEM. Statistical analysis was done with one-way ANOVA unless otherwise stated. All data expressed as mean±SEM. Statistical analysis was done with one-way ANOVA unless otherwise stated.

FIG. 5A shows Proliferation of human THP-1 and OCI-AML3 (n=22) and mouse WEHI-3B (n=8) AML cell lines exposed to SAA1 or SAA3 respectively (1 μg/ml, 24-72 h). All data expressed as mean±SEM. Statistical analysis was done with unpaired t-test unless otherwise stated.

FIG. 5B shows the proliferation levels of human bone marrow mononuclear cells (BM-MNCs) isolated from MDS or AML (lineage-depleted) BM aspirates (n=8) and exposed to SAA1 (5 μg/ml, 24 h). All data expressed as mean±SEM. Statistical analysis was done with unpaired t-test unless otherwise stated.

FIG. 5C shows IDO1 mRNA levels of human bone marrow mononuclear cells (BM-MNCs) isolated from MDS or AML (lineage-depleted) BM aspirates (n=8) and exposed to SAA1 (5 μg/ml, 24 h). All data expressed as mean±SEM. Statistical analysis was done with unpaired t-test unless otherwise stated.

FIG. 5D shows a Schematic of patient-derived xenograft (PDX) model used (left). Right: proliferation of total human BM cells isolated from the PDX mice injected with either healthy CD34+ (n=3) or patient-derived AML cells (n=8) exposed to vehicle (PBS) or SAA1 (1 μg/ml, 24 h). All data expressed as mean±SEM. Statistical analysis was done with unpaired t-test unless otherwise stated.

FIG. 5E shows IDO1 mRNA level from cells in (D); two-way ANOVA. In vivo proliferation of leukemic blasts (hCD45+CD33+). All data expressed as mean±SEM. Statistical analysis was done with unpaired t-test unless otherwise stated.

FIG. 5F shows the In vivo proliferation of leukemic blasts (hCD45⁺CD33⁺) in mice treated for 2 or 8 days with either vehicle (n=10 and n=7 respectively) or SAA1 (n=14 and n=9 respectively). All data expressed as mean±SEM. Statistical analysis was done with unpaired t-test unless otherwise stated.

FIG. 5G shows BM AML burden in mice treated for 2 or 8 days with either vehicle (n=10 and n=7 respectively) or SAA1 (n=14 and n=9 respectively). All data expressed as mean±SEM. Statistical analysis was done with unpaired t-test unless otherwise stated.

FIG. 5H shows the Proliferation of total human AML BM cells isolated from PDX mice and nucleofected with Cas9 (n=5) or Cas9 and the combination of sgRNA #126 and sgRNA #170 (n=8) exposed to vehicle or SAA1 (1 μg/ml, 24 h); two-way ANOVA. (I) mRNA level of CYP1A1 and CYP1A2 from cells in (D); two-way ANOVA. All data expressed as mean±SEM. Statistical analysis was done with unpaired t-test unless otherwise stated.

FIG. 5I shows mRNA level of CYP1A1 and CYP1A2 from cells in (D); two-way ANOVA. All data expressed as mean±SEM. Statistical analysis was done with unpaired t-test unless otherwise stated.

FIG. 5J shows Violin plots for mRNA levels of CYP1A1 and CYP1A2 in BM-MNCs from healthy (n=15) and AML (n=17) patients. All data expressed as mean±SEM. Statistical analysis was done with unpaired t-test unless otherwise stated.

FIG. 5K shows CYP1A1 and CYP1A2 mRNA levels from cells in FIG. 5(B). All data expressed as mean±SEM. Statistical analysis was done with unpaired t-test unless otherwise stated.

FIG. 5L is a GSEA analysis of AHR activation signature genes in THP-1 cells co-cultured with human osteoblasts for 24 h. All data expressed as mean±SEM. Statistical analysis was done with unpaired t-test unless otherwise stated.

FIG. 6A shows survival curve comparing vehicle (n=26), and epacadostat-treated mice (n=18 for 0.8 g/kg and n=13 for 1.6 g/kg). Kaplan-Meier curve with p-value of log rank (Mantel-Cox) test. SAA3. All data expressed as mean±SEM. Statistical analysis done with unpaired t-test unless otherwise stated.

FIG. 6B shows the SAA3/Trp ratio serum levels in NSGS mice transplanted with CD34⁺ healthy cells (n=11) or with patient-derived AML cells (n=27). All data expressed as mean±SEM. Statistical analysis done with unpaired t-test unless otherwise stated.

FIG. 6C shows the Kyn/Trp ratio serum levels in NSGS mice transplanted with CD34⁺ healthy cells (n=11) or with patient-derived AML cells (n=27). All data expressed as mean±SEM. Statistical analysis done with unpaired t-test unless otherwise stated.

FIG. 6D shows a schematic describing pharmacological targeting of IDO1 (epacadostat) in patient-derived AML xenograft (PDX) in NSGS mice. All data expressed as mean±SEM. Statistical analysis done with unpaired t-test unless otherwise stated.

FIG. 6E shows Kyn/Trp ratio in serum of PDX mice 5 weeks after AML transplant cells in the BM of PDX mice (left) and AML burden in the BM of PDX mice at harvest (right) (n=8 vehicle; n=10 epacadostat). All data expressed as mean±SEM. Statistical analysis done with unpaired t-test unless otherwise stated.

FIG. 6F is a representative flow cytometry plots depicting % of human or mouse CD45+++) of PDX mice treated with either vehicle (n=8) or epacadostat (n=8). All data expressed as mean±SEM. Statistical analysis done with unpaired t-test unless otherwise stated.

FIG. 6G is a Representative flow cytometry plots (left) and cell cycle analysis of leukemic blasts (CD45CD33 and 2 weeks post-epacadostat treatment (n=8 vehicle; n=10 epacadostat). All data expressed as mean±SEM. Statistical analysis done with unpaired t-test unless otherwise stated.

FIG. 6H shows a cell cycle analysis of mice in FIG. 6G. All data expressed as mean±SEM. Statistical analysis done with unpaired t-test unless otherwise stated.

FIG. 6I shows a schematic diagram showing the in vivo PDX mouse model treated with combination therapy (Ara-C 60 mg/kg 1-5 days+Epacadostat 1.6 g/kg ad libitum 3 weeks). AML burden in BM. All data expressed as mean±SEM. Statistical analysis done with unpaired t-test unless otherwise stated.

FIG. 6J shows a schematic diagram showing the in vivo PDX mouse model treated with the combination therapy (Ara-C 60 mg/kg 1-5 days+Epacadostat 1.6 g/kg ad libitum 3 weeks). AML burden in spleen. All data expressed as mean±SEM. Statistical analysis done with unpaired t-test unless otherwise stated.

FIG. 6K shows AML burden 11 weeks after transplant, 3 weeks after combination therapy; control chow (ctrl. n=4), Ara-C (n=3), Epacadostat (Epac. n=4) and combination therapy (Ara-C+Epac. n=3); one-way ANOVA; unpaired t-test p values are shown for BM ctrl vs Ara-C and Epac groups. All data expressed as mean±SEM. Statistical analysis done with unpaired t-test unless otherwise stated.

FIG. 6L shows Schematic model of the kynurenine-HTR1B-SAA-IDO1 axis depicting the AML-mediated osteoblastic self-reinforcing niche remodeling.

FIG. 7A shows proliferation of human AML cell lines (MOLM-14, KG-1a, Kasumi-1 and HL-60) exposed to SAA1 (1 μg/ml) for 24, 48 or 72 h, (n=8 for all cell lines); two-way ANOVA.

FIG. 7B shows AML burden, spleen weight and liver weight (over body weight) in the PDX mice 4 weeks after transplant with either CD34⁺ healthy (n=3 mice) or patient-derived AML (n=8 mice) cells.

FIG. 7C is a diagram showing the short term (2-days) vs long-term (8-days) SAA1 in vivo treatments.

FIG. 7D shows In vivo cell cycle analysis showing % of cells in G₀-G₁, G₂-M and Sub-G₁ within the leukemic blasts (hCD45⁺CD33⁺) comparing 2-day vs 8-day treatments, in vehicle-(n=10 and n=7 respectively) or SAA1-treated (0.1 mg/kg; n=14 and n=9 respectively); 2-way ANOVA. On the bottom, representative flow-plots for BM AML burden (top) and proliferation analysis (bottom) in the 8-day treatment group.

FIG. 7E shows a schematic of CRIPSR/Cas9 targeting of PDX-isolated AML human cells (left) and IDO1 mRNA level in human AML cells nucleofected with Cas9 (n=7) or Cas9 and the combination of sgRNA #126 (SEQ ID 82) and sgRNA #170 (SEQ ID 103) (n=9).

FIG. 7F shows IDO1 mRNA level of cells cultured for 24 h with either vehicle or SAA1 (1 μg/ml), (n=3); two-way ANOVA.

FIG. 7G is a schematic of Kyn treatment in low-burden PDX (left) and SAA3 serum levels in NSGS mice injected with vehicle (n=5) or Kyn (20 mg/kg; n=6) for 1 week.

FIG. 7H shows the percentage of blasts (hCD45⁺hCD33⁺) Edu⁺ cells of mice in FIG. 7G.

FIG. 7I shows AML burden in BM and SP of mice in FIG. 7G.

FIG. 7J shows mRNA level of main AHR target genes in the indicated human AML and MDS cell lines exposed to SAA1.

FIG. 7K shows mRNA level (FI over UT) of AHR targets in OCI-AML3 and THP-1 cells exposed to primary human osteoblasts for 24 h.

FIG. 8A shows Kyn/Trp ratio levels in WT mice injected or not with MLL/AF9 cells and treated with either vehicle or epacadostat (no leukemia: vehicle n=9, Epac. n=9; MLL/AF9-injected mice: vehicle n=18, Epac. n=14).

FIG. 8B shows a survival curve comparing leukemic mice treated with either vehicle (n=19) or epacadostat (n=19).

FIG. 8C shows In vivo leukemia burden quantification of mice shown in (A), treated with either vehicle or 0.8 g/kg epacadostat.

FIG. 8D shows Kyn and Trp absolute levels and Kyn/Trp ratio in serum of WT mice injected with MLL/AF9 cells and treated with either vehicle (n=6) or 1.6 g/kg ad libitum epacadostat diet (n=9).

FIG. 8E shows In vivo leukemia burden quantification of mice in (D).

FIG. 8F shows Kyn and Trp absolute levels in serum of NSGS mice transplanted with either healthy CD34⁺ cells (n=11) or patient-derived AML cells (n=27).

FIG. 8G shows a multiple variable data plot of SAA3, Kyn/Trp ratio serum levels and transplanted disease in NSGS mice transplanted with CD34⁺ healthy cells (n=11) or with patient-derived AML cells (n=27).

FIG. 8H shows AML burden in BM aspirates from PDX in NSGS mice at randomization (3 weeks; n=8 vehicle, n=10 epacadostat).

FIG. 8I shows Kyn and Trp levels in serum of PDX mice at harvest 5 weeks after transplant and 12 days post-epacadostat treatment (n=8 vehicle, n=10 epacadostat).

FIG. 8J shows AML burden in BM aspiration 8 weeks after transplant of PDX NSG mice at randomization (n=5 for all groups).

FIG. 8K shows Kyn/Trp ratio serum levels in all mice before treatment (n=20) and after 3 weeks of epacadostat diet (n=7).

### DETAILED DESCRIPTION OF CERTAIN EMBODIMENTS OF THE DISCLOSURE

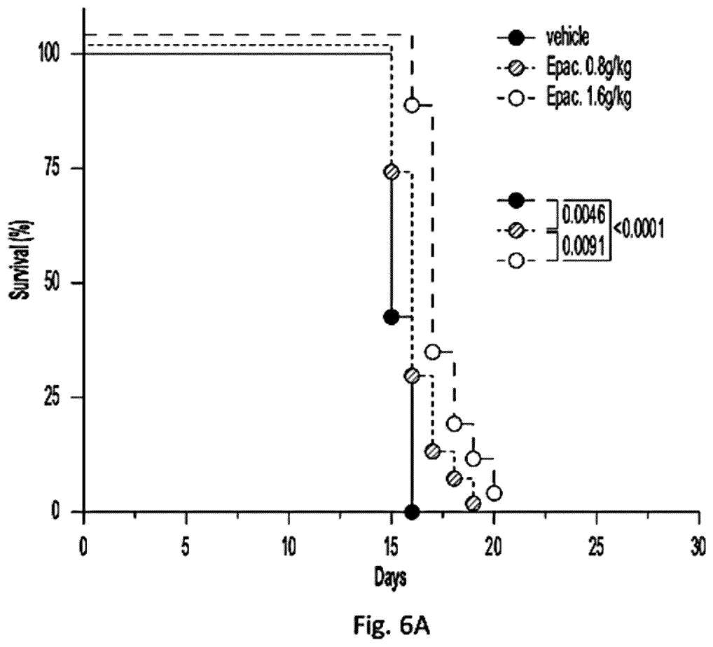

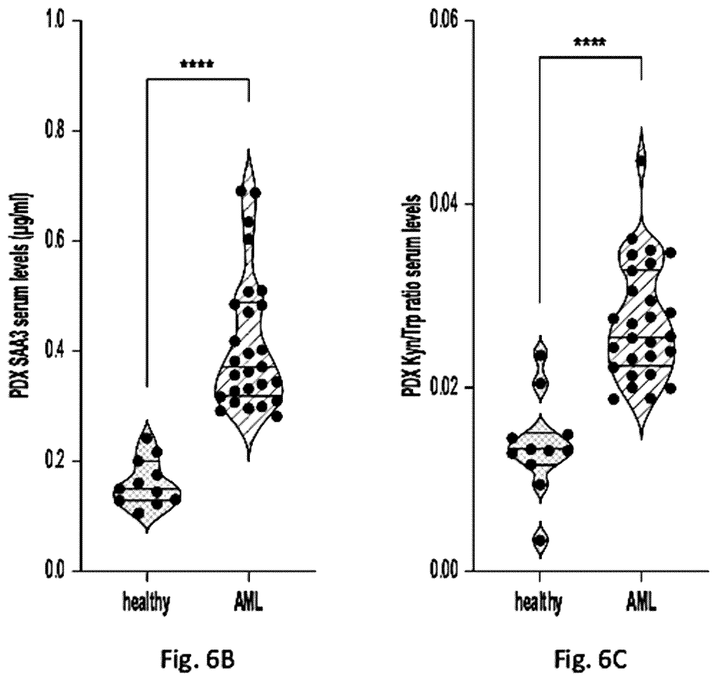

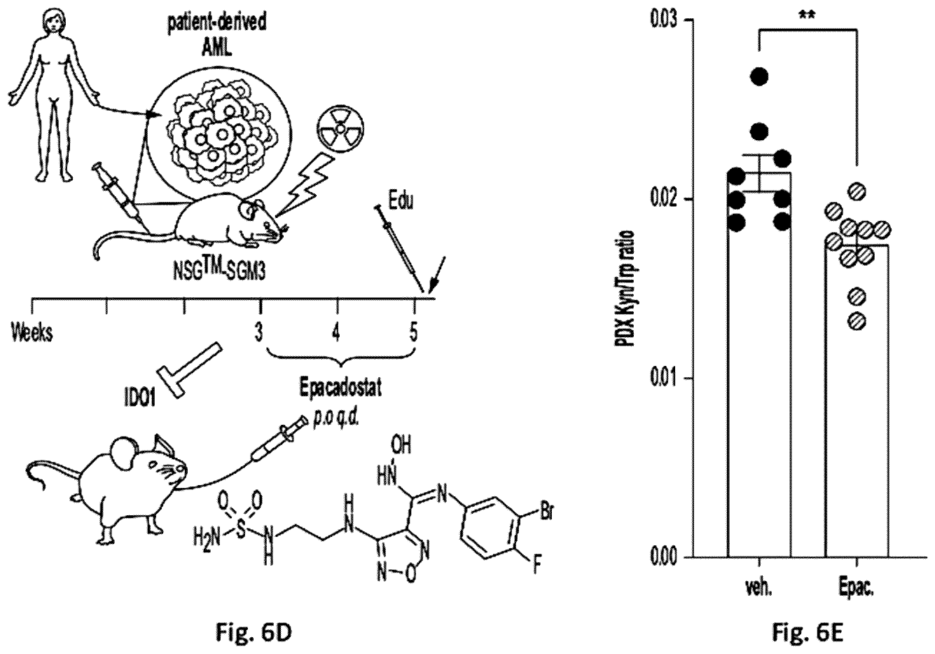

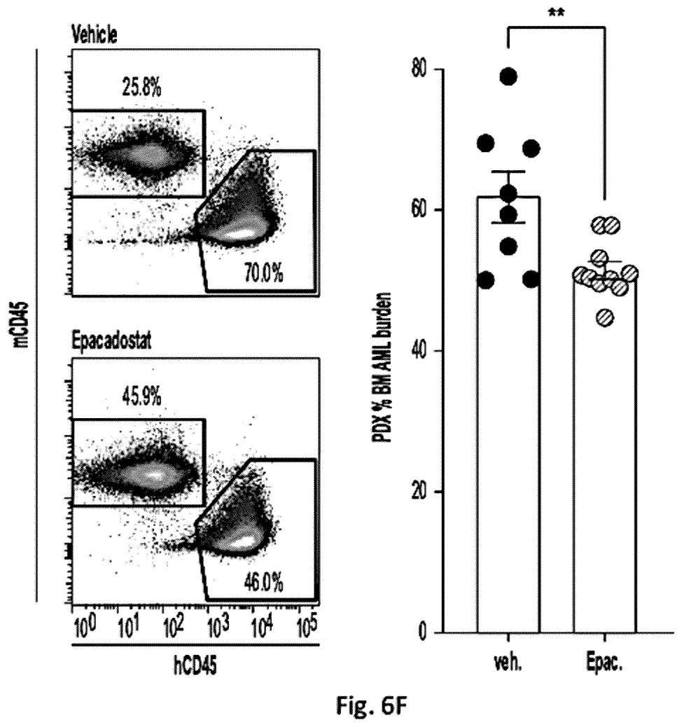

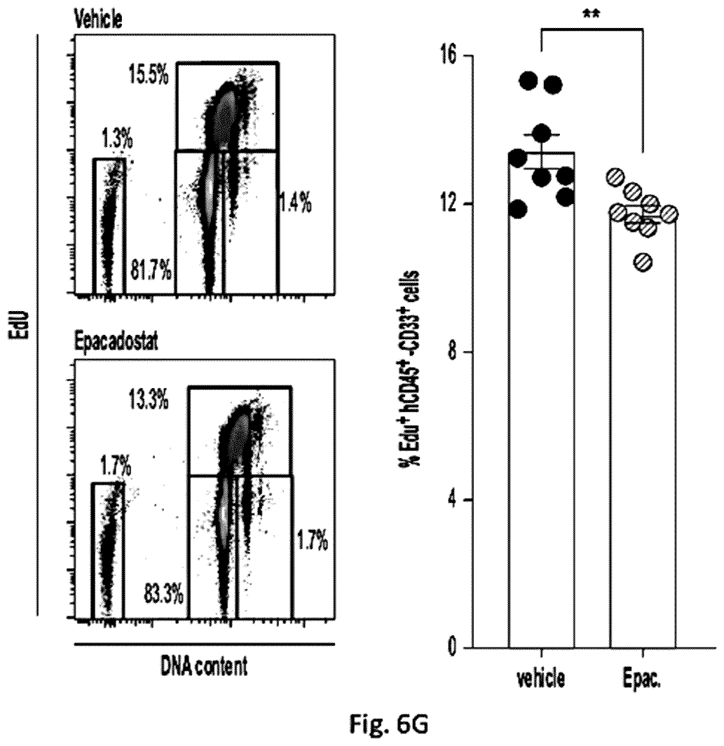

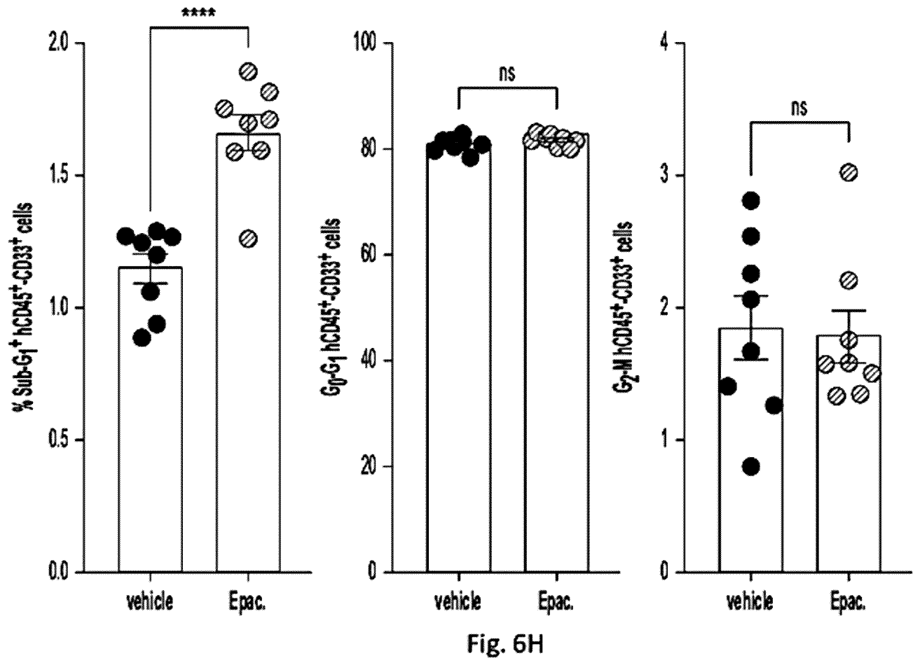

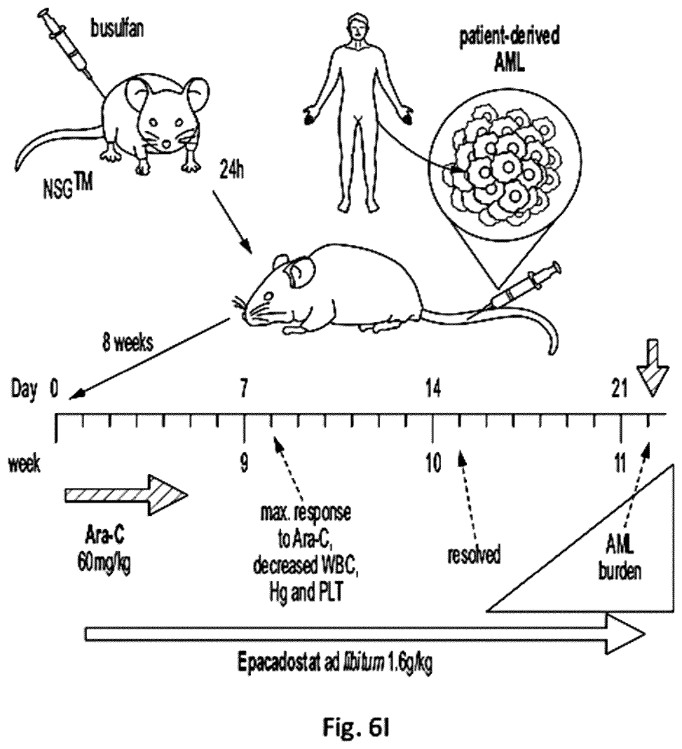

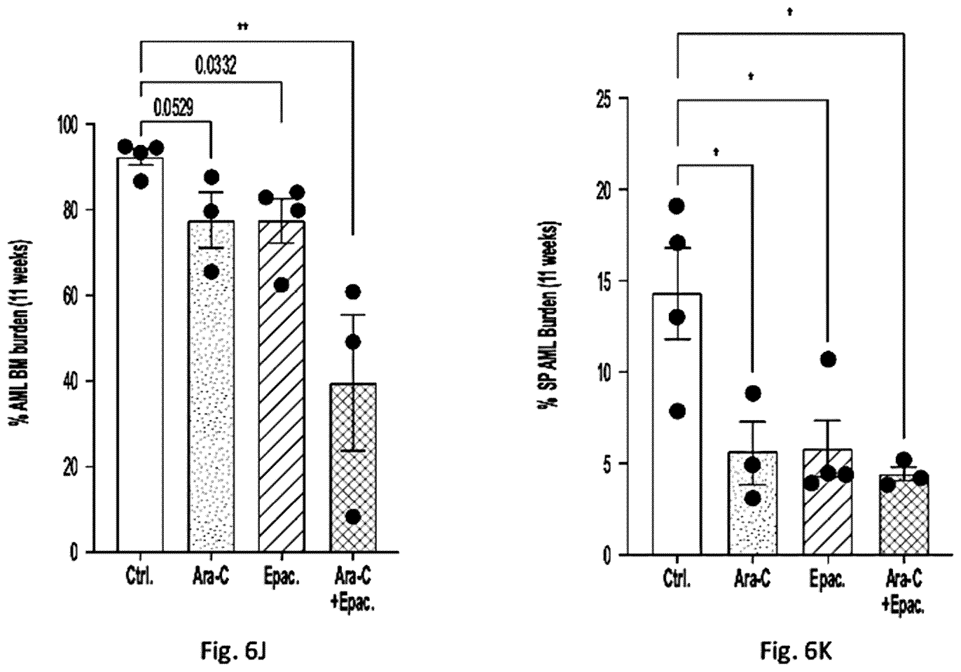

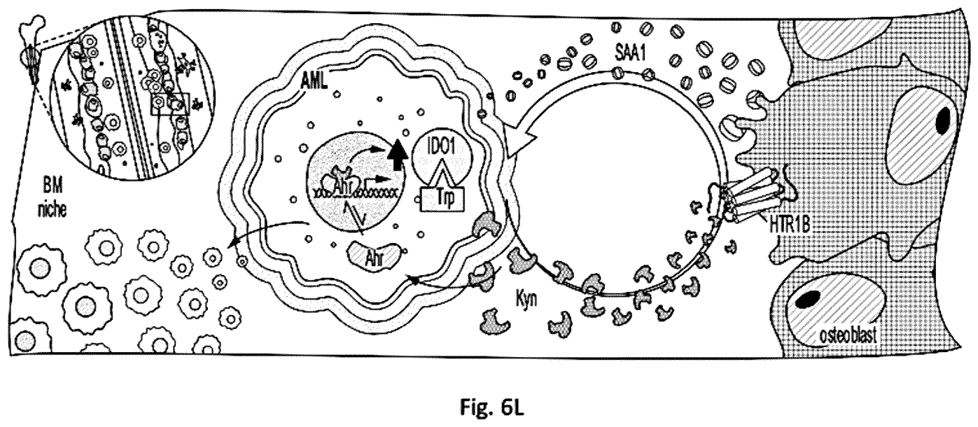

AML cells seize a peripheral serotonin signaling pathway to instruct a cycle of feedback signals in niche-osteoblasts promoting leukemia proliferation (Galan-Diez et al. Subversion of serotonin-receptor signaling in osteoblasts by kynurenine drives Acute Myeloid Leukemia. Cancer Discover 2022 12 (40): 1106-1107). (FIG. 6L). This result is achieved through the preferential production of kynurenine by AML cells, which in this setting, acts as an oncometabolite and a previously unrecognized ligand of HTR1B. Id. AML niche remodeling induces a pro-inflammatory signature in osteoblasts. Among the several pro-inflammatory molecules the expression of which is upregulated in osteoblasts, leukemia-secreted Kyn specifically induces SAA expression through HTR1B. Id. In turn, osteoblast-secreted SAA acts in AML cells to upregulate IDO1 expression, self-reinforcing leukemia proliferation. SAA1-dependent IDO1 upregulation promotes AML progression in a cell intrinsic manner by increasing kynurenine secretion (and thus activating the AHR pathway, which enhances leukemia cell proliferation), as well as by facilitating tolerance and immune escape-reviewed in Prendergast G C, et al., Discovery of IDO1 Inhibitors: From Bench to Bedside. Cancer Research. 2017; 77:6795-811.

Additionally, disruption of a specific pathway elicited by leukemia cells in osteoblasts in fact favors AML growth. A balance between those two effects allows a steady leukemia growth which eventually leads to lethality. Decreasing osteoblast numbers disrupts this balance by reducing the protective signal while the Kyn-HTR1B-SAA-IDO1 pathway is maintained and able to outweigh the weakened protective effect faster, favoring AML growth. Id.

Elevated kynurenine levels mark disease in MDS and AML patients. Id. The importance of Trp catabolismin leukemia cells is supported by other studies showing that serotonin levels are drastically decreased in MDS and AML patients as well as in leukemic mice (Ye H, et al. Subversion of Systemic Glucose Metabolism as a Mechanism to Support the Growth of Leukemia Cells. Cancer Cell. 2018; 34:659-673.e6.), and that Kyn/Trp ratios associate with several malignancies, including AML (Fukuno K, et al. Expression of indoleamine 2,3-dioxygenase in leukemic cells indicates an unfavorable prognosis in acute myeloid leukemia patients with intermediate-risk cytogenetics. Leuk Lymphoma. 2015; 56:1398-405.).

The identification of the Kyn-HTR1B-SAA-IDO1 axis in promoting AML growth, may be relevant to other cancers and could be exploited in combination with chemotherapy or immunotherapy to overcome current challenges. Lemos H, et al., Immune control by amino acid catabolismduring tumorigenesis and therapy. Nature Reviews Cancer. Nature Publishing Group; 2019; 19:162-75.

The term “modulator” refers to agents capable of modulating (e.g., down-regulating, decreasing, suppressing, or upregulating, increasing) the level/amount and/or activity of a protein, enzyme, or pathway.

The term “inhibitor” refers to agents capable of down-regulating or otherwise decreasing or suppressing the level/amount and/or activity of a protein, enzyme, or pathway.

The term “therapeutically effective amount” is an amount sufficient to treat a specified disorder or disease or alternatively to obtain a pharmacological response treating a disorder or disease.

The terms “subject,” “individual,” and “patient” are used interchangeably, and refer to a vertebrate, preferably a mammal such as a human. Mammals include, but are not limited to, human primates, non-human primates or murine, bovine, equine, canine or feline species. In the context of the present disclosure, the term “subject” also encompasses tissues and cells that can be cultured in vitro or ex vivo or manipulated in vivo. The term “subject” can be used interchangeably with the term “organism”.

The terms “polynucleotide”, “nucleotide”, “nucleotide sequence”, “nucleic acid” and “oligonucleotide” are used interchangeably. They refer to a polymeric form of nucleotides of any length, either deoxyribonucleotides or ribonucleotides, or analogs thereof. Examples of polynucleotides include, but are not limited to, coding or non-coding regions of a gene or gene fragment, exons, introns, messenger RNA (mRNA), transfer RNA, ribosomal RNA, short interfering RNA (siRNA), short-hairpin RNA (shRNA), micro-RNA (miRNA), ribozymes, cDNA, recombinant polynucleotides, branched polynucleotides, plasmids, vectors, isolated DNA of any sequence, isolated RNA of any sequence, nucleic acid probes, and primers. One or more nucleotides within a polynucleotide can further be modified. The sequence of nucleotides may be interrupted by non-nucleotide components. A polynucleotide may also be modified after polymerization, such as by conjugation with a labeling agent.

The phrase “pharmaceutically acceptable,” as used in connection with compositions and/or cells of the present disclosure, refers to molecular entities and other ingredients of such compositions that are physiologically tolerable and do not typically produce untoward reactions when administered to a mammal (e.g., a human). Preferably, as used herein, the term “pharmaceutically acceptable” means approved by a regulatory agency of the Federal or a state government or listed in the U.S. Pharmacopeia or other generally recognized pharmacopeia for use in mammals, and more particularly in humans. “Acceptable” means that the carrier is compatible with the active ingredient of the composition (e.g., the engineered exosome or extracellular vesicle) and does not negatively affect the subject to which the composition(s) are administered. The pharmaceutical compositions may comprise pharmaceutically acceptable carriers, excipients, or stabilizers in the form of lyophilized formations or aqueous solutions.

The terms “gRNA,” “guide RNA” and “CRISPR guide sequence” may be used interchangeably throughout and refer to a nucleic acid comprising a sequence that determines the specificity of a Cas DNA binding protein of a CRISPR/Cas system. A gRNA hybridizes to (complementary to, partially or completely) a target nucleic acid sequence in the genome of a host cell. The gRNA or portion thereof that hybridizes to the target nucleic acid may be between 15-25 nucleotides, 18-22 nucleotides, or 19-21 nucleotides in length. In some embodiments, the gRNA sequence that hybridizes to the target nucleic acid is 15, 16, 17, 18, 19, 20, 21, 22, 23, 24, or 25 nucleotides in length. In other embodiments, the gRNA sequence that hybridizes to the target nucleic acid is between 10-30, or between 15-25, nucleotides in length.

As used herein, a “scaffold sequence,” also referred to as a tracrRNA, refers to a nucleic acid sequence that recruits a Cas endonuclease to a target nucleic acid bound (hybridized) to a complementary gRNA sequence. Any scaffold sequence that comprises at least one stem loop structure and recruits an endonuclease may be used in the genetic elements and vectors described herein. Exemplary scaffold sequences will be evident to one of skill in the art and can be found, for example, in Jinek, et al. Science (2012) 337 (6096): 816-821, Ran, et al. Nature Protocols (2013) 8:2281-2308, PCT Application No. WO2014/093694, and PCT Application No. WO2013/176772.

“RNA interference”, or “RNAi” is a form of post-transcriptional gene silencing (“PTGS”), and comprises the introduction of, e.g., double-stranded RNA into cells (reviewed in Fire, A. Trends Genet 15:358-363 (1999); Sharp, P. Genes Dev 13:139-141 (1999); Hunter, C. Curr Biol 9: R440-R442 (1999); Baulcombe. D. Curr Biol 9: R599-R601 (1999); Vaucheret et al. Plant J 16:651-659 (1998)). The active agent in RNAi is a long double-stranded (antiparallel duplex) RNA, with one of the strands corresponding or complementary to the RNA which is to be inhibited. The inhibited RNA is the target RNA. The long double stranded RNA is chopped into smaller duplexes of approximately 20 to 25 nucleotide pairs, after which the mechanism by which the smaller RNAs inhibit expression of the target is largely unknown at this time. RNAi can work in human cells if the RNA strands are provided as pre-sized duplexes of about 19 nucleotide pairs, and RNAi worked particularly well with small unpaired 3′ extensions on the end of each strand (Elbashir et al. Nature 411:494-498 (2001)).

The invention provides for methods and compositions for treating leukemia comprising administering a therapeutically effective amount of an inhibitor of indoleamine 2,3 dioxygenase to a mammal in need thereof. In various embodiments, the mammal is a human. The leukemia may be acute myeloid leukemia or acute lymphoid leukemia. In other embodiments, the inhibitor comprises indiximod, epacadostat, BMS-986205, navoximod, PF-0684003, KHK2455 or LY3381916 or combinations thereof. In some embodiments, the inhibitor comprises epacadostat. The IDO1 inhibitor can be administered alone or in conjunction with other chemotherapeutic agents such as ARA-C. The IDO1 inhibitor can be administered orally, intravenously, intramuscularly, topically, arterially, or subcutaneously.

The invention also provides for methods and compositions for inhibiting indoleamine 2,3 dioxygenase expression comprising introducing into a eukaryotic cell an engineered, non-naturally occurring Clustered Regularly Interspaced Short Palindromic Repeats (CRISPR)-CRISPR associated (Cas) (CRISPR-Cas) system comprising one or more vectors comprising, contacting a cell with a vector comprising: a) at least one nucleotide sequence encoding a Clustered Regularly Interspaced Short Palindromic Repeats (CRISPR)-Cas system guide RNA that hybridizes with nucleotide sequences of exons 3 or 4 encoding for indoleamine 2,3 dioxygenase, and, b) a nucleotide sequence encoding a Cas protein.

The invention also provides for methods and compositions for treating leukemia in a subject, comprising administering a therapeutically effective amount of an inhibitor of indoleamine 2,3 dioxygenase to a subject. The inhibitor can bind to the enzyme catalytic site of indoleamine 2,3 dioxygenase. The inhibitor can be a small molecule, a polynucleotide, or an antibody or antigen-binding portion thereof. In certain embodiments, modulator is a nucleic acid chosen from the group consisting of a single-stranded DNA (ssDNA), a double-stranded DNA (dsDNA), a donor/template DNA, s cDNA. a DNA encoding one or more RNAs, a sgRNA, a guide RNA (gRNA), a prime editing guide RNA (pegRNA), a microRNA (miRNA) inhibitor, a miRNA mimic, a small interfering RNA (siRNA), small synthetic RNA, a synthetic RNA, an antisense oligonucleotide, a short hairpin RNA (shRNA), a double-stranded RNA (dsRNA), an antisense RNA, a ribozyme, and combinations thereof. In a preferred embodiment, the polynucleotide is a small interfering RNA (siRNA) or an antisense molecule. In another preferred embodiment, the modulator comprises a CRISPR/Cas system. The CRISPR-Cas system can be in the form of RNA, plasmid and protein. The inhibitor can be administered orally, intravenously, intramuscularly, topically, arterially, or subcutaneously, alone or in conjunction with other therapeutic agents such as ARA-C.

The invention also provides for methods and compositions for treating myelodysplastic syndrome comprising administering a therapeutically effective amount of an inhibitor of indoleamine 2,3 dioxygenase to a mammal in need thereof. The mammal can be a human. In one embodiment, the inhibitor comprises indiximod, epacadostat, BMS-986205, navoximod, PF-0684003, KHK2455 or LY3381916 or combinations thereof. The inhibitor can be administered alone or in conjunction with other therapeutic agents. In one embodiment, the inhibitor comprises epacadostat. The myelodysplastic syndrome can also be treated by introducing into a eukaryotic cell an engineered, non-naturally occurring Clustered Regularly Interspaced Short Palindromic Repeats (CRISPR)-CRISPR associated (Cas) (CRISPR-Cas) system or siRNA as described above. The inhibitor can be administered orally, intravenously, intramuscularly, topically, arterially, or subcutaneously.

The subject can be a human subject having a hematopoietic malignancy. As used herein, a hematopoictic malignancy refers to a malignant abnormality involving hematopoietic cells (e.g., blood cells, including progenitor and stem cells). Examples of hematopoietic malignancies include, without limitation, lymphoma, leukemia, or multiple myeloma. Leukemias include acute myeloid leukemia, acute lymphoid leukemia, chronic myelogenous leukemia, acute lymphoblastic leukemia or chronic lymphoblastic leukemia, chronic lymphoid leukemia as well as myelodysplastic syndromes.

The methods and compositions may be used to treat lymphoma. Non-limiting examples of lymphoma include Hodgkin's lymphoma, non-Hodgkin's lymphoma, multiple myeloma, and immunoproliferative diseases (e.g., Epstein-Barr virus-associated lymphoproliferative diseases). Non-limiting examples of lymphoma also include, relapsed or refractory lymphoma, B-cell lymphoma, T-cell lymphoma, follicular lymphoma, double-hit lymphoma, mature B cell neoplasms, mature T cell and natural killer (NK) cell neoplasms, precursor lymphoid neoplasms, immunodeficiency-associated lymphoproliferative disorders, small lymphocytic lymphoma, Burkitt's lymphoma, etc. The lymphoma may be low-grade lymphomas, intermediate-grade lymphomas, high-grade lymphomas, low-grade lymphomas.

### Inhibition by Small Molecules

The disclosure describes a peripheral serotonin-signaling axis utilized by AML cells to remodel the osteoblast niche in the bone marrow to upregulate kynurenine expression, thereby promoting AML progression and growth. Pharmacological blockade of the kynurenine synthesis pathway significantly decreases leukemia burden in the bone marrow and spleen of patient-derived xenograft models. The compositions and methods described herein to treat leukemia can be used as a standalone intervention or combination therapy with existing chemo/immunotherapies. The present methods and compositions can improve AML treatment by targeting the serotonin-signaling axis as a monotherapy or in conjunction with other regulatory approved cancer therapeutics for these diseases.

AML cells exploit serotonin receptor 1b (Htr1b) signaling in osteoblasts to proliferate. Galan-Diez et al. Subversion of serotonin-receptor signaling in osteoblasts by kynurenine drives Acute Myeloid Leukemia. Cancer Discover 2022 12 (40): 1106-1107. This proliferative pathway is not driven by serotonin (5-HT) but by another tryptophan catabolite, kynurenine, which acts as a new ligand of HTR1B in a function distinct from its reported immunoregulatory properties. Id. Using AML mouse models, patient-derived xenografts, as well as samples from AML and MDS patients, we observed that AML cells utilize kynurenine to remodel the BM niche and amplify their growth by inducing a pro-inflammatory signature in osteoblasts. Id. Among several upregulated pro-inflammatory molecules, the acute-phase protein serum amyloid A (SAA), is the signal emitted by osteoblasts that instructs AML cells to stimulate upregulation of indoleamine 2,3-dioxygenase-1 (IDO1, the rate limiting enzyme for kynurenine synthesis), selectively promoting AML proliferation. Id. Genetic and pharmacological inhibition of the kynurenine-HTR1B interaction between leukemia cells and osteoblasts significantly inhibits AML proliferation.

Inhibiting kynurenine signaling, by interrupting its binding to the serotonin receptor 1b (HTR1b), abrogates leukemia progression. Id. To assess the effects of inhibiting kynurenine synthesis in myeloid malignancies progression, we have used genetically modified as well as humanized mouse models to show that genetic ablation of the rate limiting enzyme for the synthesis of kynurenine, indoleamine 2,3-dioxygenase, hampers or even prevent leukemia progression. Id. In order to investigate the translational applicability of kynurenine synthesis inhibition, we have also pharmacologically blocked IDO1 by using an FDA-approved drug (epacadostat) in AML patient-derived xenograft models, both as a standalone intervention or as a combination therapy with 5-AZA or in combination with an antibody or reagent blocking SAA1. We have found that using epacadostat or combination of epacadostat with standard chemotherapy regiment (e.g., ARAC) significantly decreases leukemia burden in both the bone marrow and spleen. Id.

The results show that secondary recipient mice with HTR1B genetic ablation remained leukemia free after injection with MLL/AF9-induced blasts. Id. Selective IDO1 inhibition using epacadostat abrogated kynurenine secretion and impaired cell cycle progression in vitro. In vivo treatment of AML-injected mice with epacadostat led to increases in survival. In vivo treatment of wild type mice with epacadostat resulted in a 41% reduction in circulating kynurenine and tryptophan levels. Injection of AML cells with IDO1 deletion into secondary recipients significantly attenuated or abrogated disease progression. Thus, IDO1 can be an effective therapeutic target in AML. The present methods/compositions can be used as monotherapy or in combination with existing chemo/immunotherapies.

Applications of the present methods/compositions include (i) treatments for AML and/or myelodysplasia, (ii) combination therapy with chemo/immunotherapies for AML, (iii) modulating bone marrow niche interactions in the context of stem cell transplantation and immunodeficiency disorders, and (iv) improving the in vitro culturing of hematopoietic stem cells. Treatments of AML that specifically target the tumor microenvironment's contribution to AML progression, such as the osteoblastic compartment, can be effective in treating AML and improving patient outcomes.

In one embodiment, the inhibitors include one or more IDO1 inhibitors such as Indoximod (NLG8189), Epacadostat (INCB024360), Navoximod (GDC-0919) (NLG919), PF-06840003, Linrodostat (BMS-986205), NLG802, LY-3381916, LPM-3480226, HTI-1090 (SHR9146), DN1406131, or KHK2455. Sec Tang et al. J. Hematol Oncol, 2021, 14:68 and Wang et al., Expert Opinion on Therapeutic Patents, 2022, Vol. 32, No. 11, 1145-1159.

The methods and compositions may result in an inhibition of kynurenine synthesis by about 2-fold, (at least) about 3-fold, (at least) about 4-fold, (at least) about 5-fold, (at least) about 6-fold, (at least) about 7-fold, (at least) about 8-fold, (at least) about 9-fold, (at least) about 10-fold, (at least) about 1.1-fold, (at least) about 1.2-fold, (at least) about 1.3-fold, (at least) about 1.4-fold, (at least) about 1.5-fold, (at least) about 1.6-fold, (at least) about 1.8-fold, at least 2-fold, at least 3-fold, at least 4-fold, at least 5-fold, at least 6-fold, at least 7-fold, at least 8-fold, at least 9-fold, at least 10-fold, (at least) about 15-fold, (at least) about 20-fold, (at least) about 50-fold, (at least) about 100-fold, (at least) about 120-fold, from about 2-fold to about 500-fold, from about 1.1-fold to about 10-fold, from about 1.1-fold to about 5-fold, from about 1.5-fold to about 5-fold, from about 2-fold to about 5-fold, from about 3-fold to about 4-fold, from about 5-fold to about 10-fold, from about 5-fold to about 200-fold, from about 10-fold to about 150-fold, from about 10-fold to about 20-fold, from about 20-fold to about 150-fold, from about 20-fold to about 50-fold, from about 30-fold to about 150-fold, from about 50-fold to about 100-fold, from about 70-fold to about-150 fold, from about 100-fold to about 150-fold, from about 10-fold to about 100-fold, from about 100-fold to about 200-fold, of the original amount of kynurenine synthesis (in the absence of the present composition and method).

The methods and compositions may result in a decrease in kynurenine synthesis by the present composition and method that is up to 90%, up to 85%, up to 80%, up to 75%, up to 70%, up to 65%, up to 60%, up to 55%, up to 50%, up to 45%, up to 40%, up to 35%, up to 30%, up to 25%, up to 20%, up to 15%, up to 10%, about 10% to about 90%, about 15% to about 80%, about 20% to about 70%, about 25% to about 60%, about 30% to about 50%, about 30% to about 40%, about 25% to about 40%, about 20% to about 30%, about 25% to about 35%, about 10% to about 30%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, about 20% to about 50%, about 12.5% to about 80%, about 20% to about 70%, about 25% to about 60%, or about 25% to about 50%, about 1% to about 100%, about 5% to about 90%, about 10% to about 80%, about 5% to about 70%, about 5% to about 60%, about 10% to about 50%, about 15% to about 40%, about 5% to about 20%, about 1% to about 20%, about 10% to about 30%, about 5%, about 10%, about 20%, about 30%, about 40%, about 50%, about 60%, about 70%, about 80%, about 90%, about 100%, about 10% to about 90%, about 12.5% to about 80%, about 20% to about 70%, about 25% to about 60%, or about 25% to about 50%, of the original amount of kynurenine synthesis (in the absence of the present composition and method).

In various embodiments, the pharmaceutical composition may be administered intrathecally, subdurally, orally, intravenously, intramuscularly, topically, arterially, or subcutaneously. Other routes of administration of pharmaceutical compositions include oral, intravenous, subcutaneous, intramuscular, inhalation, or intranasal administration. Additionally, specifically targeted delivery of the present composition could be delivered by targeted liposome, nanoparticle or other suitable means.

The composition may be administered by bolus injection or chronic infusion. The claimed composition may be administered at or near the site of the disease, disorder or injury, in a therapeutically effective amount.

Targeted delivery of the present composition (comprising, e.g., nucleic acid, peptide, or small molecule) may be made using a targeted liposome, nanoparticle or other suitable means.

The liposomes or nanoparticles will be targeted to and taken up selectively by the desired tissue or cells.

The amount and/or activity of kynurenine synthesis may be modulated by introducing polypeptides (e.g., antibodies) or small molecules which inhibit gene expression or functional activity of the kynurenine synthesis.

Agents that bind to or modulate, such as down-regulating the amount, activity of kynurenine synthesis, may be administered to a subject or to target cells directly. Such an agent may be administered in an amount effective to down-regulate expression and/or activity of the kynurenine synthesis, or by activating or down-regulating a second signal which controls the kynurenine synthesis.

The methods and compositions may be used for prophylaxis as well as treating a disease as described herein.

The administration regimen may depend on several factors, including the serum or tissue turnover rate of the therapeutic composition, the level of symptoms, and the accessibility of the target cells in the biological matrix. Preferably, the administration regimen delivers sufficient therapeutic composition to effect improvement in the target disease state, while simultaneously minimizing undesired side effects.

An indoleamine 2,3 dioxygenase inhibitor and/or an indoleamine 2,3 dioxygenase modulator of the present invention may be present in a pharmaceutical composition in an amount ranging from about 0.005% (w/w) to about 100% (w/w), from about 0.01% (w/w) to about 90% (w/w), from about 0.1% (w/w) to about 80% (w/w), from about 1% (w/w) to about 70% (w/w), from about 10% (w/w) to about 60% (w/w), from about 0.01% (w/w) to about 15% (w/w), or from about 0.1% (w/w) to about 20% (w/w) of the total weight of the pharmaceutical composition.

An indoleamine 2,3 dioxygenase inhibitor and/or an indoleamine 2,3 dioxygenase modulator of may be present in two separate pharmaceutical compositions to be used in a combination therapy.

The pharmaceutical compositions may be administered by any route, including, without limitation, oral, transdermal, ocular, intraperitoneal, intravenous, Intracerebroventricular, intracisternal injection or infusion, subcutaneous, implant, sublingual, subcutaneous, intramuscular, intravenous, rectal, mucosal, ophthalmic, intrathecal, intra-articular, intra-arterial, sub-arachinoid, bronchial and lymphatic administration. The pharmaceutical composition may be administered parenterally or systemically.

The pharmaceutical compositions of the present invention can be, e.g., in a solid, semisolid, or liquid formulation. Intranasal formulation can be delivered as a spray or in a drop; inhalation formulation can be delivered using a nebulizer or similar device; topical formulation may be in the form of gel, ointment, paste, lotion, cream, poultice, cataplasm, plaster, dermal patch aerosol, etc.; transdermal formulation may be administered via a transdermal patch or iontophoresis. Pharmaceutical compositions can also take the form of tablets, pills, capsules, semisolids, powders, sustained release formulations, solutions, emulsions, suspensions, elixirs, aerosols, chewing bars or any other appropriate compositions.

The pharmaceutical composition may be administered locally via implantation of a membrane, sponge, or another appropriate material on to which the desired molecule has been absorbed or encapsulated. Where an implantation device is used, the device may be implanted into any suitable tissue or organ, and delivery of the desired molecule may be via diffusion, timed release bolus, or continuous administration.

To prepare such pharmaceutical compositions, one or more of compounds of the present invention may be mixed with a pharmaceutical acceptable excipient, e.g., a carrier, adjuvant and/or diluent, according to conventional pharmaceutical compounding techniques.

Pharmaceutically acceptable carriers that can be used in the present compositions encompass any of the standard pharmaceutical carriers, such as a phosphate buffered saline solution, water, and emulsions, such as an oil/water or water/oil emulsion, and various types of wetting agents. The compositions can additionally contain solid pharmaceutical excipients such as starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Liquid carriers, particularly for injectable solutions, include water, saline, aqueous dextrose, and glycols. For examples of carriers, stabilizers, preservatives and adjuvants, see Remington's Pharmaceutical Sciences, edited by E. W. Martin (Mack Publishing Company, 18th ed., 1990). Additional excipients, for example sweetening, flavoring and coloring agents, may also be present.

The pharmaceutically acceptable excipient may be selected from the group consisting of fillers, e.g. sugars and/or sugar alcohols, e.g. lactose, sorbitol, mannitol, maltodextrin, etc.; surfactants, e.g. sodium lauryl sulfate, Brij 96 or Tween 80; disintegrants, e.g. sodium starch glycolate, maize starch or derivatives thereof; binder, e.g. povidone, crosspovidone, polyvinylalcohols, hydroxypropylmethylcellulose; lubricants, e.g. stearic acid or its salts; flowability enhancers, e.g. silicium dioxide; sweeteners, e.g. aspartame; and/or colorants. Pharmaceutically acceptable carriers include any and all clinically useful solvents, dispersion media, coatings, antibacterial and antifungal agents, isotonic and absorption delaying agents and the like.

The pharmaceutical composition may contain excipients for modifying, maintaining or preserving, for example, the pH, osmolarity, viscosity, clarity, color, isotonicity, odor, sterility, stability, rate of dissolution or release, adsorption or penetration of the composition. Suitable excipients include, but are not limited to, amino acids (such as glycine, glutamine, asparagine, arginine or lysine); antimicrobials; antioxidants (such as ascorbic acid, sodium sulfite or sodium hydrogen sulfite); buffers (such as borate, bicarbonate, Tris HCl, citrates, phosphates, other organic acids); bulking agents (such as mannitol or glycine), chelating agents (such as ethylenediamine tetraacetic acid (EDTA), ethylene glycol tetraacetic acid (EGTA)); complexing agents (such as caffeine, polyvinylpyrrolidone, beta cyclodextrin or hydroxypropyl beta cyclodextrin); fillers; monosaccharides; disaccharides and other carbohydrates (such as glucose, mannose, or dextrins); proteins (such as serum albumin, gelatin or immunoglobulins); coloring; flavoring and diluting agents; emulsifying agents; hydrophilic polymers (such as polyvinylpyrrolidone); low molecular weight polypeptides; salt forming counterions (such as sodium); preservatives (such as benzalkonium chloride, benzoic acid, salicylic acid, thimerosal, phenethyl alcohol, methylparaben, propylparaben, chlorhexidine, sorbic acid or hydrogen peroxide); solvents (such as glycerin, propylene glycol or polyethylene glycol); sugar alcohols (such as mannitol or sorbitol); suspending agents; surfactants or wetting agents (such as pluronics, PEG, sorbitan esters, polysorbates such as polysorbate 20, polysorbate 80, triton, tromethamine, lecithin, cholesterol, tyloxapal); stability enhancing agents (sucrose or sorbitol); tonicity enhancing agents (such as alkali metal halides (in one aspect, sodium or potassium chloride, mannitol sorbitol); delivery vehicles; diluents; excipients and/or pharmaceutical adjuvants. (Remington's Pharmaceutical Sciences, 18th Edition, A. R. Gennaro, ed., Mack Publishing Company, 1990).

Oral dosage forms may be tablets, capsules, bars, sachets, granules, syrups and aqueous or oily suspensions. Tablets may be formed form a mixture of the active compounds with fillers, for example calcium phosphate; disintegrating agents, for example maize starch, lubricating agents, for example magnesium stearate; binders, for example microcrystalline cellulose or polyvinylpyrrolidone and other optional ingredients known in the art to permit tableting the mixture by known methods. Similarly, capsules, for example hard or soft gelatin capsules, containing the active compound, may be prepared by known methods. The contents of the capsule may be formulated using known methods so as to give sustained release of the active compounds. Other dosage forms for oral administration include, for example, aqueous suspensions containing the active compounds in an aqueous medium in the presence of a non-toxic suspending agent such as sodium carboxymethylcellulose, and oily suspensions containing the active compounds in a suitable vegetable oil, for example arachis oil. The active compounds may be formulated into granules with or without additional excipients. The granules may be ingested directly by the patient, or they may be added to a suitable liquid carrier (e.g., water) before ingestion. The granules may contain disintegrants, e.g., an effervescent pair formed from an acid and a carbonate or bicarbonate salt to facilitate dispersion in the liquid medium. U.S. Pat. No. 8,263,662.

Intravenous forms include, but are not limited to, bolus and drip injections. Examples of intravenous dosage forms include, but are not limited to, Water for Injection USP; aqueous vehicles including, but not limited to, Sodium Chloride Injection, Ringer's Injection, Dextrose Injection, Dextrose and Sodium Chloride Injection, and Lactated Ringer's Injection; water-miscible vehicles including, but not limited to, ethyl alcohol, polyethylene glycol and polypropylene glycol; and non-aqueous vehicles including, but not limited to, corn oil, cottonseed oil, peanut oil, sesame oil, ethyl oleate, isopropyl myristate and benzyl benzoate.

Additional pharmaceutical compositions include formulations in sustained or controlled delivery, such as using liposome or micelle carriers, bioerodible microparticles or porous beads and depot injections.

The compound(s) or pharmaceutical composition may be administered as a single dose, or as two or more doses (which may or may not contain the same amount of the desired molecule) over time, or as a continuous infusion via implantation device or catheter. The pharmaceutical composition can be prepared in single unit dosage forms.

Appropriate frequency of administration can be determined by one of skill in the art and can be administered once or several times per day (e.g., twice, three, four or five times daily). The compositions of the invention may also be administered once each day or once every other day. The compositions may also be given twice weekly, weekly, monthly, or semi-annually. In the case of acute administration, treatment is typically carried out for periods of hours or days, while chronic treatment can be carried out for weeks, months, or even years. U.S. Pat. No. 8,501,686.

Administration of the compositions of the invention can be carried out using any of several standard methods including, but not limited to, continuous infusion, bolus injection, intermittent infusion, inhalation, or combinations of these methods. For example, one mode of administration that can be used involves continuous intravenous infusion. The infusion of the compositions of the invention can, if desired, be preceded by a bolus injection.

Methods of determining the most effective means and dosage of administration can vary with the composition used for therapy, the purpose of the therapy, the target cell being treated, and the subject or patient being treated. Single or multiple administrations can be carried out with the dose level and pattern being selected by the treating physician. The specific dose level for any particular subject depends upon a variety of factors including the activity of the specific peptide, the age, body weight, general health, sex, diet, time of administration, route of administration, and rate of excretion, drug combination and the severity of the particular disease undergoing therapy.

For example, an indoleamine 2,3 dioxygenase inhibitor and/or an indoleamine 2,3 dioxygenase modulator may be administered at about 0.0001 mg/kg to about 500 mg/kg, about 0.01 mg/kg to about 200 mg/kg, about 0.01 mg/kg to about 0.1 mg/kg, about 0.1 mg/kg to about 100 mg/kg, about 10 mg/kg to about 200 mg/kg, about 10 mg/kg to about 20 mg/kg, about 5 mg/kg to about 15 mg/kg, about 0.0001 mg/kg to about 0.001 mg/kg, about 0.001 mg/kg to about 0.01 mg/kg, about 0.01 mg/kg to about 0.1 mg/kg, about 0.1 mg/kg to about 0.5 mg/kg, about 0.5 mg/kg to about 1 mg/kg, about 1 mg/kg to about 2.5 mg/kg, about 2.5 mg/kg to about 10 mg/kg, about 10 mg/kg to about 50 mg/kg, about 50 mg/kg to about 100 mg/kg, about 100 mg/kg to about 250 mg/kg, about 0.1 μg/kg to about 800 μg/kg, about 0.5 μg/kg to about 500 μg/kg, about 1 μg/kg to about 20 μg/kg, about 1 μg/kg to about 10 μg/kg, about 10 μg/kg to about 20 μg/kg, about 20 μg/kg to about 40 μg/kg, about 40 μg/kg to about 60 μg/kg, about 60 μg/kg to about 100 μg/kg, about 100 μg/kg to about 200 μg/kg, about 200 μg/kg to about 300 μg/kg, or about 400 μg/kg to about 600 μg/kg. In some embodiments, the dose is within the range of about 250 mg/kg to about 500 mg/kg, about 0.5 mg/kg to about 50 mg/kg, or any other suitable amounts.

The therapeutically effective amount of the indoleamine 2,3 dioxygenase inhibitor and/or an indoleamine 2,3 dioxygenase modulator of the present invention for the combination therapy may be less than, equal to, or greater than when the agent is used alone.

The amount or dose of an indoleamine 2,3 dioxygenase inhibitor and/or an indoleamine 2,3 dioxygenase modulator may range from about 0.01 mg to about 10 g, from about 0.1 mg to about 9 g, from about 1 mg to about 8 g, from about 1 mg to about 7 g, from about 5 mg to about 6 g, from about 10 mg to about 5 g, from about 20 mg to about 1 g, from about 50 mg to about 800 mg, from about 100 mg to about 500 mg, from about 600 mg to about 800 mg, from about 800 mg to about 1 g, from about 0.01 mg to about 10 g, from about 0.05 μg to about 1.5 mg, from about 10 μg to about 1 mg protein, from about 0.1 mg to about 10 mg, from about 2 mg to about 5 mg, from about 1 mg to about 20 mg, from about 30 μg to about 500 μg, from about 40 μg to about 300 pg, from about 0.1 μg to about 200 mg, from about 0.1 μg to about 5 μg, from about 5 μg to about 10 μg, from about 10 μg to about 25 μg, from about 25 μg to about 50 μg, from about 50 μg to about 100 μg, from about 100 μg to about 500 μg, from about 500 μg to about 1 mg, from about 1 mg to about 2 mg.

Different dosage regimens may be used. In some embodiments, a daily dosage, such as any of the exemplary dosages described above, is administered once, twice, three times, or four times a day for at least three, four, five, six, seven, eight, nine, or ten days. Depending on the stage and severity of the cancer, a shorter treatment time (e.g., up to five days) may be employed along with a high dosage, or a longer treatment time (e.g., ten or more days, or weeks, or a month, or longer) may be employed along with a low dosage. In some embodiments, a once- or twice-daily dosage is administered every other day.

The invention provides for a method of inhibiting indoleamine 2,3 dioxygenase expression comprising introducing into a eukaryotic cell an engineered, non-naturally occurring Clustered Regularly Interspaced Short Palindromic Repeats (CRISPR)-CRISPR associated (Cas) (CRISPR-Cas) system comprising one or more vectors comprising a) a first regulatory element operable in a eukaryotic cell operably linked to at least one nucleotide sequence encoding a CRISPR-Cas system guide RNA that hybridizes with sequences encoding exons 3 or 4 of indoleamine 2,3 dioxygenase, and b) a second regulatory element operable in a eukaryotic cell operably linked to a nucleotide sequence encoding a Cas9 protein, wherein components (a) and (b) are located on same or different vectors of the system, whereby the guide RNA targets sequences encoding exons 3 or 4 of indoleamine 2,3 dioxygenase and the Cas9 protein cleaves the DNA molecule, whereby expression of indoleamine 2,3 dioxygenase protein is reduced; and, wherein the Cas9 protein and the guide RNA do not naturally occur together.

The Cas enzyme may be a type II, type I, type III, type IV or type V CRISPR system enzyme. In some embodiments, the Cas enzyme is a Cas9 enzyme (also known as Csn1 and Csx12). Cas9 may be wild-type or mutant. In certain embodiments, the Cas enzyme is Cas9, Cpf1, C2c1, C2c2, C2c3, Cas1, Cas1β, Cas2, Cas3, Cas4, Cas5, Cas6, Cas7, Cas8, Cas10, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, Csf4, homologs thereof, orthologs thereof, or modified versions thereof. In one embodiment, the Cas enzyme is Cas9.

CRISPR interference (CRISPRi) or CRISPR activation (CRISPRa) may be used in the present systems and methods. CRISPRi is a transcriptional interference technique that allows for sequence-specific repression of gene expression and/or epigenetic modifications in cells (Qi et al., (2013) Repurposing CRISPR as an RNA-guided platform for sequence-specific control of gene expression. Cell 152 (5): 1173-83). CRISPRi regulates gene expression primarily on the transcriptional level. CRISPRi can sterically repress transcription, e.g., by blocking transcriptional initiation or elongation. The target sequence may be the promoter and/or exonic sequences (such as the non-template strand and/or the template strand), and/or introns (Ji et al., (2014). Specific gene repression by CRISPRi system transferred through bacterial conjugation. ACS Synthetic Biology 3 (12): 929-31). CRISPRi can also repress transcription via an effector domain. Fusing a repressor domain to a catalytically inactive Cas enzyme, e.g., dead Cas9 (dCas9), may further repress transcription. For example, the Krüppel associated box (KRAB) domain can be fused to dCas9 to repress transcription of the target gene (Gilbert et al., 2013, CRISPR-mediated modular RNA-guided regulation of transcription in eukaryotes. Cell 154 (2): 442-51).

In one embodiment, the IDO1 inhibitor can be a nucleic acid such as a single-stranded DNA (ssDNA), a double-stranded DNA (dsDNA), a donor/template DNA, scDNA. a DNA encoding one or more RNAs, a sgRNA, a guide RNA (gRNA), a prime editing guide RNA (pegRNA), a microRNA (miRNA) inhibitor, a miRNA mimic, a small interfering RNA (siRNA), small synthetic RNA, a synthetic RNA, an antisense oligonucleotide, a short hairpin RNA (shRNA), a double-stranded RNA (dsRNA), an antisense RNA, a ribozyme, and combinations thereof. In some embodiments, the polynucleotide is a small interfering RNA (siRNA) or an antisense molecule. In a preferred embodiment, the inhibitor comprises a CRISPR/Cas system. The CRISPR-Cas system can be in the form of RNA, plasmid and protein. The nuclei acids can be administered to the subject via any route described herein.

The present methods may utilize adeno-associated virus (AAV) mediated gene delivery. Additionally, delivery vehicles such as nanoparticle- and lipid-based nucleic acid or protein delivery systems can be used as an alternative to viral vectors. Further examples of alternative delivery vehicles include lentiviral vectors, lipid-based delivery system, gene gun, hydrodynamic, electroporation or nucleofection microinjection, and biolistics. Various gene delivery methods are discussed in detail by Nayerossadat et al. (Adv Biomed Res. 2012; 1:27) and Ibraheem et al. (Int J Pharm. 2014 Jan. 1; 459 (1-2): 70-83).

The present methods may use nanoparticle-based siRNA delivery systems. The nanoparticle-formulated siRNA delivery systems may be based on polymers or liposomes. Nanoparticles conjugated to the cell-specific targeting ligand for effective siRNA delivery can increase the chance of binding the cell surface receptor. The nanoparticles may be coated with PEG (polyethylene glycol) which can reduce uptake by the reticuloendothelial system (RES), resulting in enhanced circulatory half-life. Various nanoparticle-based delivery systems such as cationic lipids, polymers, dendrimers, and inorganic nanoparticles may be used in the present methods to provide effective and efficient siRNA delivery in vitro or in vivo.

The vectors may be delivered into host cells by a suitable method. Methods of delivering the present composition to cells may include transfection of nucleic acids or polynucleotides (e.g., using reagents such as liposomes or nanoparticles); electroporation, delivery of protein, e.g., by mechanical deformation (see, e.g., Sharei et al. Proc. Natl. Acad. Sci. USA (2013) 110 (6): 2082-2087); or viral transduction. Exemplary viral vectors include, but are not limited to, recombinant retroviruses, alphavirus-based vectors, and adeno-associated virus (AAV) vectors. In some embodiments, the vectors are retroviruses. In one embodiment, the vectors are lentiviruses. In another embodiment, the vectors are adeno-associated viruses.

The vectors described herein can be transformed, transfected or otherwise introduced into a wide variety of host cells. Numerous methods of transfection are known to the ordinarily skilled artisan, for example, lipofectamine, calcium phosphate co-precipitation, electroporation, DEAE-dextran treatment, microinjection, viral transduction, and other methods known in the art. Transduction refers to entry of a virus into the cell and expression (e.g., transcription and/or translation) of sequences delivered by the viral vector genome. In the case of a recombinant vector, “transduction” generally refers to entry of the recombinant viral vector into the cell and expression of a nucleic acid of interest delivered by the vector genome.

The CRISPR (Clustered Regularly interspaced Short Palindromic Repeats) system exploits RNA-guided DNA-binding and sequence-specific cleavage of target DNA. A guide RNA (gRNA) is complementary to a target DNA sequence. The guide RNA/Cas combination confers site specificity to the nuclease. A single guide RNA (sgRNA) contains about 20 nucleotides that are complementary to a target genomic DNA sequence and a constant RNA scaffold region. The Cas (CRISPR-associated) protein binds to the guide RNA (gRNA) or sgRNA and the target DNA to which the gRNA or sgRNA binds and introduces a double-strand break. Geurts et al., Science 325:433 (2009); Mashimo et al., PLOS ONE 5:e8870 (2010); Carbery et al., Genetics 186:451-459 (2010); Tesson et al., Nat. Biotech. 29:695-696 (2011). Wiedenheft et al. Nature 482:331-338 (2012); Jinek et al. Science 337:816-821 (2012); Mali et al. Science 339:823-826 (2013); Cong et al. Science 339:819-823 (2013).

In addition to a sequence that binds to a target nucleic acid, in some embodiments, the gRNA also comprises a scaffold sequence. Expression of a gRNA encoding both a sequence complementary to a target nucleic acid and scaffold sequence has the dual function of both binding (hybridizing) to the target nucleic acid and recruiting the endonuclease to the target nucleic acid, which may result in site-specific CRISPR activity. In some embodiments, such a chimeric gRNA may be referred to as a single guide RNA (sgRNA).

Cleavage of a gene region may comprise cleaving one or two strands at the location of the target sequence by the Cas enzyme. In one embodiment, such, cleavage can result in decreased transcription of a target gene. In another embodiment, the cleavage can further comprise repairing the cleaved target polynucleotide by homologous recombination with an exogenous template or donor DNA, wherein the repair results in an insertion, deletion, or substitution of one or more nucleotides of the target polynucleotide.

In some embodiments, the gRNA sequence does not comprise a scaffold sequence and a scaffold sequence is expressed as a separate transcript. In such embodiments, the gRNA sequence further comprises an additional sequence that is complementary to a portion of the scaffold sequence and functions to bind (hybridize) the scaffold sequence and recruit the endonuclease to the target nucleic acid.

In some embodiments, the gRNA sequence is at least 50%, 55%, 60%, 65%, 70%, 75%, 80%, 85%, 90%, 95%, 96%, 97%, 98%, 99%, or at least 100% complementary to a target nucleic acid (see also U.S. Pat. No. 8,697,359, which is incorporated by reference for its teaching of complementarity of a gRNA sequence with a target polynucleotide sequence).

A gRNA can have a length ranging from about 12 nucleotides to about 100 nucleotides. For example, gRNA can have a length ranging from about 12 nucleotides (nt) to about 80 nt, from about 12 nt to about 50 nt, from about 12 nt to about 40 nt, from about 12 nt to about 30 nt, from about 12 nt to about 25 nt, from about 12 nt to about 20 nt, or from about 12 nt to about 19 nt. For example, the first segment (e.g., crRNA) can have a length of from about 19 nt to about 20 nt, from about 19 nt to about 25 nt, from about 19 nt to about 30 nt, from about 19 nt to about 35 nt, from about 19 nt to about 40 nt, from about 19 nt to about 45 nt, from about 19 nt to about 50 nt, from about 19 nt to about 60 nt, from about 19 nt to about 70 nt, from about 19 nt to about 80 nt, from about 19 nt to about 90 nt, from about 19 nt to about 100 nt, from about 20 nt to about 25 nt, from about 20 nt to about 30 nt, from about 20 nt to about 35 nt, from about 20 nt to about 40 nt, from about 20 nt to about 45 nt, from about 20 nt to about 50 nt, from about 20 nt to about 60 nt, from about 20 nt to about 70 nt, from about 20 nt to about 80 nt, from about 20 nt to about 90 nt, or from about 20 nt to about 100 nt. A gRNA can have fewer than 12 nucleotides or greater than 100 nucleotides.

sgRNA(s) can be between about 5 and 100 nucleotides long, or longer (e.g., 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59 60, 61, 62, 63, 63, 64, 65, 66, 67, 68, 69, 70, 71, 72, 73, 74, 75, 76, 77, 78, 79, 80, 81, 82, 83, 84, 85, 86, 87, 88, 89, 90, 91 92, 93, 94, 95, 96, 97, 98, 99, or 100 nucleotides in length, or longer). In one embodiment, sgRNA(s) can be between about 15 and about 30 nucleotides in length (e.g., about 15-29, 15-26, 15-25; 16-30, 16-29, 16-26, 16-25; or about 18-30, 18-29, 18-26, or 18-25 nucleotides in length).

### Inhibitory Nucleic Acids

In certain embodiments, the cargo or payload may be an inhibitory nucleic acid or polynucleotide that reduces expression of a target gene. Thus, the polynucleotide specifically targets a nucleotide sequence encoding a target protein or polypeptide.

The nucleic acid target of the polynucleotides may be any location within the gene or transcript of the target protein or polypeptide.

The inhibitory nucleic acids may be RNA interference or RNAi, an antisense RNA, a ribozyme, or combinations thereof.

RNAi may be small interfering RNA or siRNAs, a small hairpin RNA or shRNAs, microRNA or miRNAs, a double-stranded RNA (dsRNA), etc.

The cargo or payload may be a short RNA molecule, such as a short interfering RNA (siRNA), a small temporal RNA (stRNA), and a micro-RNA (miRNA). Short interfering RNAs silence genes through an mRNA degradation pathway, while stRNAs and miRNAs are approximately 21 or 22 nt RNAs that are processed from endogenously encoded hairpin-structured precursors, and function to silence genes via translational repression. See, e.g., McManus et al., RNA, 8 (6): 842-50 (2002); Morris et al., Science, 305 (5688): 1289-92 (2004); He and Hannon, Nat Rev Genet. 5 (7): 522-31 (2004).

Alternatively, a polynucleotide encoding an siRNA or shRNA may be used.

The inhibitory nucleic acids may be an antisense nucleic acid sequence that is complementary to a target region within the mRNA of a target protein or polypeptide. The antisense polynucleotide may bind to the target region and inhibit translation. The antisense oligonucleotide may be DNA or RNA or comprise synthetic analogs of ribo-deoxynucleotides.

An antisense oligonucleotide can be, for example, about 7, 10, 15, 20, 25, 30, 35, 40, 45, 50, 55, 60, 65, 70, 75, 80, or more nucleotides in length.

The cargo or payload may be a ribozyme. Ribozymes can be chemically synthesized and structurally modified to increase their stability and catalytic activity using methods known in the art.

### Antibodies

The cargo or payload may be an antibody or a fragment (e.g., an antigen-binding portion) thereof.

The antibody or antigen-binding portion thereof may be the following: (a) a whole immunoglobulin molecule; (b) a single-chain variable fragment (scFv); (c) a Fab fragment; (d) an F(ab′)2; and (e) a disulfide linked Fv. The antibody or antigen-binding portion thereof may be monoclonal, polyclonal, chimeric and humanized. The antibodies may be murine, rabbit or human/humanized antibodies.

### EXAMPLES

The below examples and data are exemplary and are non-limiting. See also, Galan-Diez et al. Subversion of serotonin-receptor signaling in osteoblasts by kynurenine drives Acute Myeloid Leukemia. Cancer Discover 2022 12 (40): 1106-1107. Which is incorporated herein in its entirety by reference.

### Example 1: Mice

Wilt type (WT) C57BL/6J (IMSR Cat #JAX: 000664, RRID: IMSR_JAX: 000664), BALB/cJ (IMSR Cat #JAX: 000651, RRID: IMSR_JAX: 000651), NOD.Cg-Prkdescid Il2rgtm1 Wjl/SzJ (NSG, IMSR Cat #JAX: 005557, RRID: IMSR_JAX: 005557) and NOD.Cg-Prkdcscid Il2rgtm1 Wjl Tg (CMV-IL3,CSF2,KITLG) 1Eav/MloySzJ (NSGS, IMSR Cat #JAX: 013062, RRID: IMSR_JAX: 013062) mice were purchased from Jackson Laboratories. All other animals used in the study were bred in our mouse facility, kept in a C57BL/6J background and used between 8-10 weeks old. Male and female mice were used indistinctly. Htr1b−/− mice were obtained from Dr. Rene Hen at Columbia University (Saudou F, et al. Enhanced aggressive behavior in mice lacking 5-HT1B receptor. Science. American Association for the Advancement of Science; 1994; 265:1875-8.). Htr1bfl/fl mice were obtained from Dr. Greengard at Rockefeller University (Virk M S, et al. Opposing roles for serotonin in cholinergic neurons of the ventral and dorsal striatum. Proceedings of the National Academy of Sciences. National Acad Sciences; 2016; 113:734-9.) and were mated to LepRCre, Col1a-Cre (33), OCN-Cre lines (34) or Osx-Cre (Rodda S J, et al. Distinct roles for Hedgehog and canonical Wnt signaling in specification, differentiation and maintenance of osteoblast progenitors. Development. Oxford University Press for The Company of Biologists Limited; 2006; 133:3231-44.) to generate homozygous mice lacking Htr1b in the indicated tissues. The Osx-Cre mice were kept on doxycycline-containing diet (0.625 g/kg), DOX was removed in the experimental group 24 h after MLL/AF9 injection. All mouse genetic models were used with their respective WT littermates as controls. Experimental animals have been maintained at the Columbia University animal facility under specific pathogen-free and in accordance with Institutional Animal Care and Use Committee (IACUC) of Columbia University approved protocols.

PTH bone-anabolic treatment: mice were injected intraperitoneally (i.p.) with PTH (Bachem) at 80 μg/kg/day in PBS. Injections started 1 week before MLL/AF9 injection and continued along 2-3 more weeks until mice were harvest.

SB224289-SB9-(TOCRIS Cat #1221) treatment: mice were injected intraperitoneally (i.p.) daily with SB9 (5 mg/kg in 0.9% NaCl) 1 week after leukemia injection and for the duration of the experiment. Assuming a 20 g body weight (BW) and a 2 ml total blood volume per mouse—as well as an even distribution of the drug-systemic concentration of SB9 should be approximately 50 μg/ml. Based on the MW of SB9 (557.09), the final concentration—at equal distribution—in blood should be 8.97521e-05 M (≈90 μM).

Epacadostat (AdooQ Cat #A15554)-treatment: for the WT C57BL/6J mice, treatment started at the same time than MLL/AF9 cells were transplanted. For the patient-derived AML cells (PDX) transplanted into NSG mice, treatment started 8 weeks after transplant, at the same time than Ara-C and during 3 weeks. Mice were supplied with ad libitum epacadostat-supplemented diet (Research Diets Inc.) at 800 mg/kg (low-dose) or 1.6 g/kg (high-dose). For the PDX transplanted into NSGS mice, treatment started 3 weeks after transplant, by daily gavage at 300 mg/kg (InvivoChem Cat #: V0942, dissolved in 10% DMSO, 40% PEG 300 and 50% NaCl 0.9%) for 2 weeks.

SAA1 (Peprotech Cat #300-53) treatment: for short-term treatment (2 days), mice were injected intra venous (i.v.) 72 h and 48 h before harvesting. For long-term treatment (8-days), mice received daily i.v. injections. In order to get the same SAA1 concentration in blood that the one used in vitro, we used a dosage of 100 μg/kg of SAA1, diluted in 0.9% NaCl. Assuming a 20 g body weight, 2 ml total blood volume, and an even distribution in the mouse, systemic concentration of SAA1 should be approximately 1 μg/ml.

Serum for ELISA analysis was collected from cardiac puncture, left untouched for 30 min at RT and centrifuged 15 min at 4° C. 12.000 rpm; samples were snap-frozen in liquid nitrogen and stored at −80° C. until further analysis.

Complete blood counts were assessed on cardiac-puncture peripheral blood (at harvest/end-point) collected into EDTA-coated tubes (Becton Dickinson) using a Genesis (Oxford Science) hematology system.

### Example 2: Patient Samples

Primary MDS and AML patient's samples: Bone marrow (BM) aspirate samples and bone biopsies from male and female MDS and AML patients between the age of 53-87 were obtained from an Institutional Review Board (IRB)-approved tissue repository at the Myelodysplastic Syndromes Center at New York Presbyterian-Columbia University Medical Center. 3-10 ml of BM aspirate were collected from the iliac crest of the back of the hip bone. 0.5-1 ml was used for BM plasma collection (15 min at 2000 g's 4° C.), snap-freezed in liquid nitrogen and stored at −80° C. until analysis. The study populations reflected the populations usually seen at the clinics at Columbia University Medical Center. Those include 60% males, 40% females with 60% Caucasian, 30% Hispanic, 10% African Americans and Non-Hispanic. MDS and AML are predominantly a disease of elderly (median age at diagnosis: 74 years). Less than 15% of the patients with MDS are between the ages of 18-65 and greater than 85% will be above age 65.

BM samples from the University of Pennsylvania were obtained from the Stem Cell and Xenograft Core. The Core has maintained an IRB approved protocol for 20 years. All samples were obtained as de-identified and previously collected. As with CUMC, the race and sex of samples in the Core reflects that of the patient population seen at the Hospital of the University of Pennsylvania.

Healthy biopsies: healthy BM aspirates and bone biopsies were obtained from the Orthopedic Surgery Department at Columbia University, in collaboration with Dr. R. Shah. Healthy patients who have a planned elective hip or knee surgery were asked by about their participation in the study, reflecting surgeries of men (44%) or women (56%) with ages ranging between 18-65 years old (46%) and >65 (54%).

All studies were approved by the Columbia University Medical Center Institutional Review Board (IRB Protocol Numbers: AAAK3058 and AAAR3184) and informed written consent was obtained from all participants. Research was conducted in compliance with the declaration of Helsinki for collection and use of sample materials in research protocols, and in compliance with IRB regulations. Isolation of BM mononuclear cells was performed by density gradient centrifugation using Ficoll-Paque standard procedures.

### Example 3: Cell Lines and Primary Cell Cultures

OCI-AML3 (DSMZ Cat #ACC-582, RRID: CVCL_1844), THP-1 (DSMZ Cat #ACC-16, RRID: CVCL_0006) and MOLM-14 (DSMZ Cat #ACC-777, RRID: CVCL_7916) cells were acquired from the DSMZ repository. SC (ATCC Cat #CRL-9855, RRID: CVCL_6444), HL-60 (ATCC Cat #CCL-240, RRID: CVCL_0002), MV4-11 (ATCC Cat #CRL-9591, RRID: CVCL_0064), KG-1a (ATCC Cat #CCL-246.1, RRID: CVCL_1824), Kasumi-1 (ATCC Cat #CRL-2724, RRID: CVCL_0589) and HEK293T (ATCC Cat #CRL-3216, RRID: CVCL_0063) cells were obtained from the ATCC and WEHI-3B (ECACC Cat #86013003, RRID: CVCL_2239) from Sigma. The MDS-L cell line was a kind gift from Dr. Amit K. Verma (Albert Einstein College of Medicine). Cell lines not directly obtained from their source were validated via short tandem repeat DNA profiling. All cell lines were routinely tested for Mycoplasma (Venor™ GeM Mycoplasma Detection Kit, Sigma-Aldrich Cat #MP0025).

OCI-AML3 and THP-1 cell lines as well as primary human osteoblasts were grown in MEM-Alpha 1× (Corning); HEK293T cells were grown in DMEM (Corning); SC, HL-60, MOLM-14, KG-1a, Kasumi-1 and MV4-11 were grown in IMDM (Gibco). The MDS-L cell line was grown in RPMI supplemented with 1× beta-mercaptoethanol and IL-3 (10 μg/ml). All media was supplemented with 10% FBS (Gibco, except primary human osteoblasts, OCI-AML3 and HL-60 that needed 20%, 1% GlutaMAX (Gibco) and 1% antibiotic-antimycotic (Corning) and cultured at 37° C. with 5% CO₂.

MLL/AF9 primary cells were maintained in StemSpan medium (StemCell Technologies) containing mGM-CSF (10 ng/ml), mSCF (25 ng/ml), mIL-6 (25 ng/ml), mIL-3 (10 ng/ml), mTPO (25 ng/ml) (Prepotech) and 1% P/S.

Human primary MDS and/or AML cells: patient-derived AML cells for CRISPR experiments, were cultured with Stemspan II (Stemcell Tech), 1% PS, completed with 100 ng/ml of human FLT3L and SCF, 50 ng/ml of human TPO, IL3 and IL6 (BioLegend) and 750 nM of SR1 (Cayman Chemical). For the ex vivo cultures, AML and/or MDS cells were cultured on StemMACS HSC Expansion Media XF supplemented with StemMACS HSC Expansion Cocktail (Miltenyi Biotec).

Primary human osteoblasts were obtained from explants of healthy patients undergoing hip/knee replacement surgery. Outgrowth cultures yielded osteoblastic stromal cells that were differentiated in osteogenic media (5 mM β-glycerol phosphate and 100 μg/ml ascorbic acid; Sigma) changed every other day for 10-13 days.

Primary calvaria-derived osteoblasts were prepared from calvaria of 2-3 day-old newborns as previously described (Rached M-T, et al. FoxO1 Is a Positive Regulator of Bone Formation by Favoring Protein Synthesis and Resistance to Oxidative Stress in Osteoblasts. Cell Metabolism. Elsevier Ltd; 2010; 11:147-60.). Briefly, mice calvaria were sequentially digested for 20, 40, and 90 min at 37° C. in alpha-MEM (Gibco) 10% FBS containing 0.1 mg/ml of collagenase P (Worthington) and 0.25% trypsin (Gibco). Cells of the first two digests were discarded, whereas cells released from the third digestion were plated and differentiated for 7-10 days as previously described.

Co-cultures were set up using a 0.4 μm-pore transwell (Falcon), with primary osteoblasts on the bottom compartment and the leukemic cells on upper one. Both cells were starved overnight (o/n) and co-cultured together in alpha-MEM for the indicated period of time in an osteoblast-to-leukemia ratio of 1:10.

Treatments with recombinant proteins: human IL-1α, IL-1β, IL-6, IL-33, IL-34, CXCL1, CXCL3, CXCL5, CXCL8, CCL2, CCL20, Apo-SAA1 (all from Peprotech) and recombinant mouse SAA3 (Cusabio) were performed by o/n treatment with 50 ng/ml of the corresponding protein. SAA1 treatment of human AML cell lines was done with 1 μg/ml for 24, 48 or 72 h. Treatment of primary human MDS or AML lineage-depleted BM-MNCs was done with 5 μg/ml for 24 h. Treatment of PDX isolated human total BM cells was done with 1 μg/ml for 24 h.

### Example 4: Leukemic Syngeneic Mouse Models and Assessment of Leukemia In Vivo Progression

All leukemia models were introduced by intravenous (i.v.) injection and transplanted into non-irradiated secondary recipient experimental animals. BALB/c mice were used for the WEHI-3B leukemia model (0.5×10⁶/cells/mouse) and C57BL/6J mice for MLL/AF9-dsRed (0.2×10⁶/cells/mouse). Leukemia progression was assessed by fluorescence (MLL/AF9 dsRed) using the IVIS-Spectrum Optical Imaging System (Caliper, Perkin Elmer). Mice were shaved to reduce light attenuation.

### Example 5: Xenograft Models

4-6 weeks old NSG (CDX model) or NSGS (PDX model) mice were pre-conditioned with sublethal (1.4 Gy) total-body irradiation. 24 h after, 1×10⁶ OCI-AML3 or 2×10⁵ human BM CD34⁺ (healthy) or primary AML patient samples were injected i.v. Engraftment levels were monitored and mice were randomized after BM aspiration 3-4 weeks later and immunophenotyped by the presence of mCD45 (BioLegend Cat #103133, RRID: AB_10899570), hCD45 (BioLegend Cat #368512, RRID: AB_2566372), hCD33 (BioLegend Cat #303404, RRID: AB_314348), hCD34 (BioLegend Cat #343518, RRID: AB_1937203) cell populations. For the low-burden PDX model (kynurenine injections), mice were treated 1 week after transplant.

For the combination therapy (Epacadostat+chemotherapy) performed at the University of Pennsylvania, patient-derived AML cells were transplanted as previously reported (48). Briefly 6 weeks old NSG males were sublethally treated with busulfan (30 mg/kg) 24 h before transplant and 5×10⁶ patient-derived AML cells were injected i.v. Engraftment was assessed, and mice were randomized at 7.5 weeks by BM aspirate as previously described. Randomized mice were treated with vehicle, cytosine arabinoside (Ara-C, 60 mg/kg/day×5 days i.p.), epacadostat chow (1.6 g/kg ad libitum) or both Ara-C and epacadostat chow for 3 weeks.

### Example 6: Immunofluorescence Staining

Tissue: after harvesting, spleen and liver were fixed o/n in 4% PFA, washed with PBS and kept on a 30% sucrose gradient for at least 16 h before OCT. For bones, fixation was done for 72 h following 7 days decalcification on 14% EDTA pH7 before sucrose gradient and OCT embedding. All tissues were cut using a Leyca cryostat, dried at RT and stored at −80° C. Sections were rehydrated in PBS for 10 min and stained with DAPI. Cells: osteoblasts were grown over 12 mm coverslips, differentiated and exposed for 30-60 min to conditioned media from OCI-AML3 cells at a 1:10 ratio, fixed in 4% PFA 15 min RT, permeabilized (PBS 0.3% Triton X-100) 15 min RT, blocked (PBS 5% donkey normal serum, 0.3% Triton X-100) and stained o/n at 4° C. with p65 (Cell Signaling Technology Cat #8242, RRID: AB_10859369) and DAPI (nuclei). Slides were mounted with anti-fade Prolong Gold (Invitrogen) mounting-medium, and images acquired on a Zeiss LSM 710 confocal microscope. Images were analyzed with ImageJ (RRID:SCR_003070) software.

### Example 7: Metabolomics

Cell culture supernatant samples (150 μl) were loaded into Ostro Protein Precipitation & Phospholipid Removal Plate (Waters: 186005518). 20 μl internal standards and 450 μL of acetonitrile (0.2% formic acid) were added. After pressure pushing through the plate, the samples were transferred to a new vial, and dry under gentle nitrogen flow. The samples were reconstituted to 100 μl of 80% methanol-20% water for analysis with ABsciex 6500+ with Ace PFP column. A pooled quality control (QC) sample was injected ×6 for coefficient of variation (CV) calculation. Metabolites with CVs<20% are considered as accurate quantification, while CVs>35% are treated as poorly-accurate results. PCA 2D scores plot was calculated to show the degree of overlap between the three data point clusters in PC scores space. PLS-DA scores plot was calculated with PC1 representing the difference between the 3 groups and PC2 differences between the co-cultures and the AML. Analysis of metabolomic data was performed on Matplotlib for Python.

### Example 8: Liquid Chromatography-Mass Spectrometry (LC-MS)

Cell culture supernatant samples were analyzed at the Biomarkers Core Laboratory (BCL) of Columbia University by targeted LC-MS based assays for the biogenic amines Tryptophan (Trp), Kynurenine (Kyn) and serotonin (5-HT).

### Example 9: Kynurenine, Tryptophan, SAA3 and SAA1 Serum/Plasma Levels

Quantification in serum from peripheral blood (mice) or BM plasma (patients) of Kyn and Trp was assessed by ELISA using independent kits (ImmunoSmol) as per manufacturer's instructions. The ratio between Kyn and Trp levels is shown. SAA3 in serum (murine SAA3 ELISA Kit Millipore) and SAA1 in patient BM plasma (Amyloid A1 DuoSet ELISA Kit (R&D) were assessed as per manufacturer's instructions.

### Example 10: Total RNA Extraction and RT-qPCR Gene Expression Analysis

RNA isolation, cDNA preparation and real-time PCR analyses were carried out following standard protocols. Total RNA from cortical bone (clean, flushed femurs, were centrifugated 20 seconds at 10.000 g's to remove any remaining BM) was extracted using TRIzol (Invitrogen) followed by RNA clean-up using PureLink RNA Mini Kit (Ambion, Invitrogen). mRNA was reversed transcribed using random hexamers RNA-to-cDNA kit (Takara). Specific forward and reverse primers were used in conjunction with PowerUp SYBR Green Master Mix (Applied Biosystems) for quantitative PCR. Expression levels were analyzed using the 2^{-ΔΔCt} method and were normalized for the expression of the housekeeping gene Hprt unless otherwise stated.

### Example 11: Radioligand Binding Assays

The full-length murine or human serotonin receptor 1b (Htr1b) (pCMV6-Entry vector, Myc-DDK-tagged, Origene, Cat #MR222524 and RC223874 respectively) were transiently transfected into HEK293T cells using Lipofectamine LTX (Invitrogen). Transfection efficiency was assessed 24 h post-transfection by flow cytometry using the anti-Flag antibody (Sigma-Aldrich Cat #F3165, RRID: AB_259529). Binding of 25 nM [³H]-5-HT (41.3 Ci/mmol, Perkin Elmer) or [³H]-Kyn (50 μM, 0.125 Ci/mmol), was performed with 100 μg of isolated HEK293 membranes in a final volume of 50 μl of binding buffer (10 mM Hepes, pH 7.4, 100 mM NaCl, 10 mM MgCl₂, 1% ascorbic acid, 1× entacapone/pargyline), incubated for 3 h at 4° C. in the presence of varying concentrations of non-labelled additions (5-HT, Kyn or SB9). Reactions were stopped by the addition of ice-cold PBS, filtered through 0.7 μm glass fiber filters (Data Support Company). Filters were dried and melted with scintillation cocktail. Radioactivity captured on the filters was counted using a SL300 scintillation counter (Hidex). Unspecific binding of [³H]-5-HT or [³H]-Kyn in the presence or absence of each compound with the glass filters was determined in the absence of membranes; specific binding was determined by subtracting the unspecific binding signal from that measured in the presence of the HTR1B-expressing membranes in the appropriate conditions. Log EC₅₀ were determined by Non-linear regression curve analysis.

[³H]-GR125743 (PerkinElmer) radioligand binding assays were performed in standard binding buffer (50 mM Tris, 10 mM MgCl₂, 0.1 mM EDTA, 0.1% BSA, 0.01% ascorbic acid, pH 7.4). Competitive binding was assessed with various concentrations of test compounds (0.3 nM to 100 μM), [³H]-GR125743 (1.38 nM), and HTR1B membranes (isolated from HEK293T stable transfectants) in a total volume of 150 μL. Assay plates were incubated in the dark for 1 h at RT and reactions were stopped by filtration onto 0.3% polyethyleneimine pre-soaked 96-well Filtermat A (PerkinElmer), followed with three quick washes with cold wash buffer (50 mM Tris, pH 7.4). Filters were dried and melted with scintillation cocktail (Meltilex, PerkinElmer). Radioactivity was counted using a Wallac TriLux Microbeta counter (PerkinElmer).

### Example 12: CAMP Signaling Assays

The GloSensor cAMP assays were conducted as previously reported (Patel N, et al. Structure-based discovery of potent and selective melatonin receptor agonists. Elife. 2020; 9) with minor modifications. Briefly, HEK293T were transiently co-transfected with 4 μg of 5-HT_{1B} receptor and 4 μg of GloSensor cAMP (Promega) plasmids o/n and plated in Poly-L-Lysine coated 384-well white clear bottom plates in DMEM supplemented with 1% dialyzed FBS for 24 h. Cells were removed of the culture medium and loaded with luciferin (final of 1 mM) for 30 min at 37° C. The cells were then stimulated with the drugs diluted in assay buffer (HBSS, 20 mM HEPES, 1 mg/ml BSA, pH 7.4) for 15 min at RT, followed by addition of isoproterenol (100 nM). The plates were counted in a Wallac TriLux Microbeta counter (PerkinElmer) after 25 min.

### Example 13: Proliferation Assays

Cell proliferation was performed by using Cell Counting Kit 8 (WST-8, Abcam) as per manufacturer's instructions. Briefly, 0.03×10⁶ cells were seeded on tissue-culture clear bottom microplates (Corning) in their corresponding media (100 μl). When indicated, cells were treated with the indicated compounds and for the indicated time points. 10 μl/well of WST-8 solution was added and incubated for 2 h at 37° C. before measuring absorbance at 460 nm. For each experiment, the absorbance of the blank wells (growth media and vehicle/treatment) was subtracted from the values for those wells with cells.

In vitro: the indicated cell lines were incubated in reduced-serum media and exposed to SAA1 (1 μg/ml) for the 24-72 h as indicated.

Ex vivo xenografts (healthy CD34⁺ versus patient-derived AML): total BM from NSGS mice was depleted of mouse cells with mouse CD45 magnetic beads (Miltenyi Biotec Cat #130-052-301, RRID: AB_2877061) and negatively selected human cells used.

Ex vivo primary AML and MDS patient's samples: MNCs from fresh BM patients' aspirates were isolated as previously described and depleted from mature hematopoietic cells (lineage Cell Depletion Kit, Miltenyi Biotec Cat #130-092-211). Isolated cells were seeded on StemMACS HSC Expansion Media XF supplemented with StemMACS HSC Expansion Cocktail (Miltenyi Biotec, Cat #130-100-463 & 130-100-843) and treated with either vehicle (PBS) or SAA1 (5 μg/ml) for 24 h.

Invitrogen Violet Annexin V/Dead Cell Apoptosis Kit (catalog no. A35136) was used to assess the fate of cells.

### Example 14: In Vivo Proliferation (Edu) Cell Cycle Analysis

Cell labelling was done by i.p. injection of mice with 50 mg/kg of freshly prepared 5-Ethynyl-2′-deoxyuridine-Edu-(Cayman Chemical Company Cat #20518). After 3-4 h, BM was harvested, and human cells were negatively selected by mouse cell depletion using mouse CD45 magnetic beads (Miltenyi). The human BM cells were then stained CD45 and CD33 to identify the leukemic blast. Cell cycle/proliferation analysis was performed using the Click-iT Plus EdU Flow Cytometry Assay Kit (Invitrogen, Cat #C10420) following manufacturers' instructions. Fixable viability dye (Biolegend) was used to discriminate the death population. Single color controls were used to set compensations and fluorescence minus one control were used to set gates. Analysis was performed with FlowJo software.

### Example 15: CRISPR/Cas9-Mediated Ido1 Genomic Targeting

The chemically modified sgRNAs targeting IDO1 were obtained and designed with at least 3 mismatches to decrease possible off target effects with the Synthego CRISPR design tool or the CRISPOR. Analysis of the predicted coding protein genes for each sgRNA did not reveal enrichment for any specific pathway or cellular process, especially no gene signature associated to TP53 or DNA damage pathways were identified. In addition, lack of random effects due to TP53 activation, was shown by p16 and p21 mRNA level assessment in Cas9-only controls as well as in all the sgRNAs used. For 10⁶ cells, 3 μg of TrueCut Cas9 protein V2 (Invitrogen) and 1.5 μg sgRNA were mixed in either SE (immortalized cell lines WEHI-3B and OCI-AML3) or P3 (primary mouse MLL/AF9 or patient-derived AML cells) buffer (Lonza, Amaxa X-Nucleofector Kit) and incubated 10 mins. Cells were then resuspended in their respective nucleofection buffer, mixed with the Cas9/sgRNA RNP complex or the Cas9 only as control, and electroporated with the Lonza 4D-Nucleofector (program DZ100, CM137 or DI100). After electroporation, cells were cultured in their respective media at 37° C. until sequencing analysis and/or injection. The editing efficiency data, indel contribution and Sanger sequence analyses were performed with the Synthego Performance Analysis, ICE Analysis. 2019. v2.0. (Synthego).

Table 3 shows the Off-target sites for mouse sgRNA 146 (PAM in bold): CGCCAUGGUGAUGUACCCCA GGG (SEQ ID 1). Mismatches with guide sequence are shown in bold and underlined. Off target sites located in non-coding regions are indicated by an empty box in the Gene column.

Table 4 shows the Off-target sites for mouse sgRNA 196 (PAM in bold): CUGCCCACACUGAGCACGGA CGG (SEQ ID 22). Mismatches with guide sequence are shown in bold and underlined. Off target sites located in non-coding regions are indicated by an empty box in the Gene column.

Table 5 shows Off-target sites for mouse sgRNA 203 (PAM in bold): CAGUCCGUCCGUGCUCAGUG TGG (SEQ ID 41). Mismatches with guide sequence are shown in bold and underlined. Off target sites located in non-coding regions are indicated by an empty box in the Gene column.

Table 6 shows Off-target sites for mouse sgRNA 610 (PAM in bold): UAGGGAACAGCAAUAUUGCG GGG (SEQ ID 61). Mismatches with guide sequence are shown in bold and underlined. Off target sites located in non-coding regions are indicated by an empty box in the Gene column.

Table 7 shows Off-target sites for human sgRNA 126 (PAM in bold): GUGCAAGGCGCUGUGACUUG TGG (SEQ ID 82). Mismatches with guide sequence are shown in bold and underlined. Off target sites located in non-coding regions are indicated by an empty box in the Gene column.

Table 8 shows Off-target sites for human sgRNA 170 (PAM in bold): UUUGCCCCACACAUAUGCCA UGG (SEQ ID 103). Mismatches with guide sequence are in bold and underlined. Off target sites located in non-coding regions are indicated by an empty box in the Gene column.

### Example 16: Plasmid Constructs and Lenti-Viral Transduction

Lentiviral particles were obtained by co-transfection of Lenti-X™ Packaging Single Shots (VSV-G) (Takara Bio Cat #631275) and either empty or pLenti-IDO1-C-mGFP Vector (Origene Cat #RC206592L2) in HEK293T cells, according to the manufacturer's protocol. Supernatants containing the viral particles were concentrated using PEG Virus Precipitation Kit (BioVision, Cat #K904) according to the manufacturer's protocol. Viral titers were quantified using Lenti-X™ GoStix™ Plus (Takara Bio Cat #631280). 2×10⁶ OCI-AML3 cells were transduced with the indicated multiplicity of infection (MOI) by spinoculation (300×g for 1 hr at 32° C.) in the presence of 8 μg/ml Polybrene (Milipore) 24 h before assessment of proliferation.

### Example 17: RNA Sequencing (RNAseq)

Briefly, total RNA was extracted from primary human osteoblasts and THP-1 cells co-cultured with the transwell device using TRIzol. Paired-end transcriptome reads were processed using STAR (Dobin A, et al. STAR: ultrafast universal RNA-seq aligner. Bioinformatics. 2013; 29:15-21) aligner based on the Ensembl (RRID:SCR_002344) GRCh37 human genome assembly with default parameters. Read count values were extracted using featureCounts (Liao Y, et al. featureCounts: an efficient general purpose program for assigning sequence reads to genomic features. Bioinformatics. 2014; 30:923-30) and normalized gene expression were calculated as TPM (Transcripts Per Million). Differential expression analysis was performed by DEseq2 (RRID:SCR_015687) (Love M I, et al., Moderated estimation of fold change and dispersion for RNA-seq data with DEScq2. Genome Biol. 2014; 15:550). The RNA sequencing data are deposited in GEO (GSE154374).

### Example 18: Multiplex Analysis of Protein Levels

Cell culture supernatants were probed for: IL-1alpha, IL-6, CXCL1, CXCL5, CXCL8, CCL2, CCL7, CCL8 and CCL20 using a custom-made multiplex panel (Invitrogen ProcartaPlex) per manufacturing instructions. Supernatant samples were clarified by centrifugation at 10,000 g for 10 min and kept on ice prior loading.

### Example 19: Osteoblasts Inhibit AML by a Mechanism Involving Serotonin Signaling

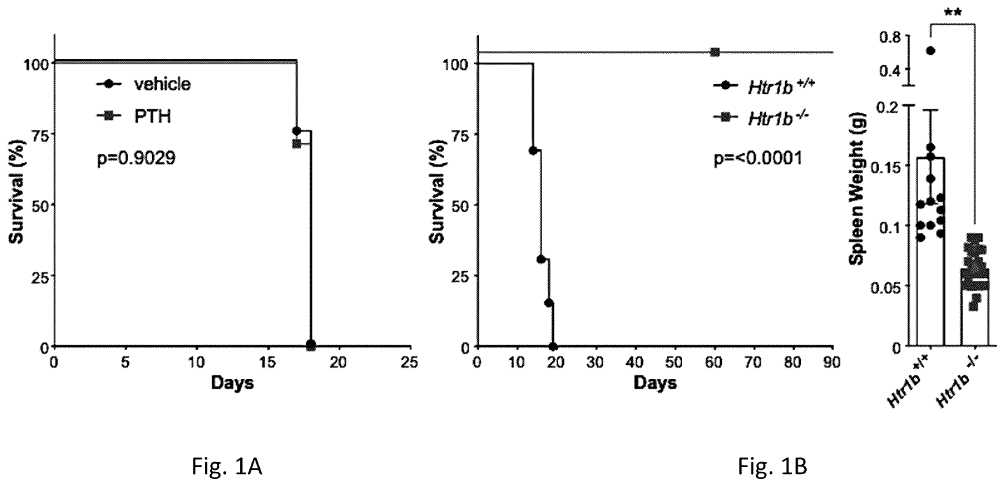

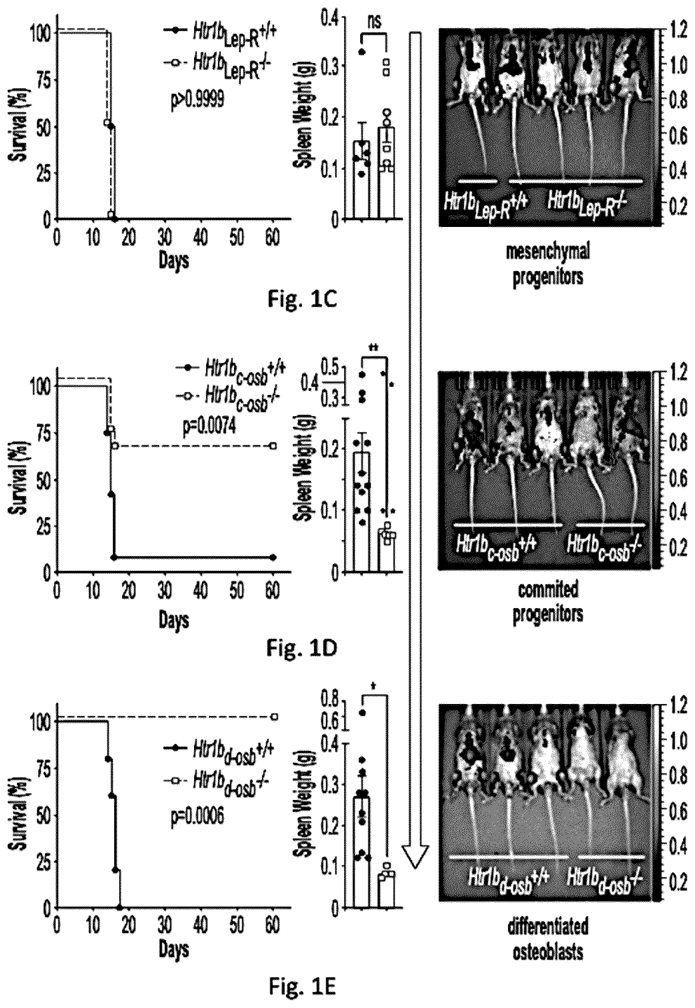

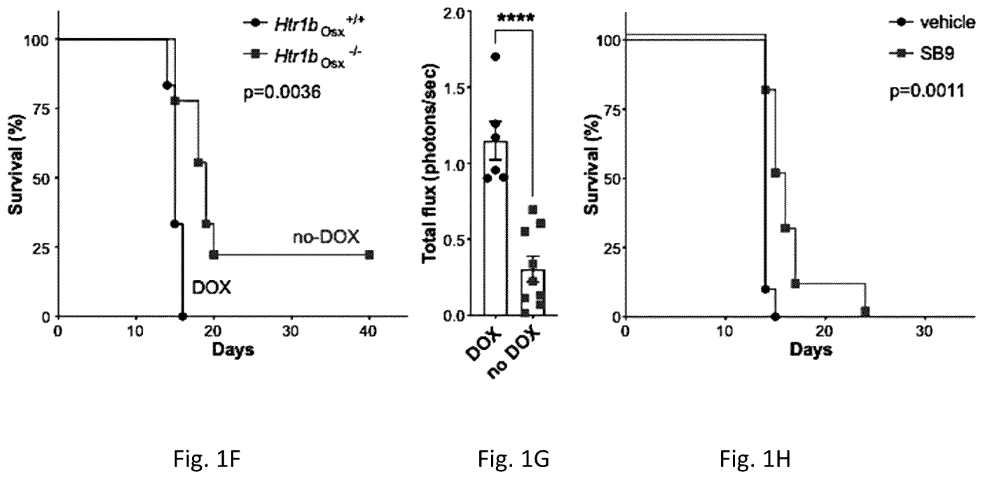

We have previously shown that the maintenance of osteoblast numbers by inhibiting anti-proliferative actions of gut-derived serotonin reduces leukemia burden and prolongs survival (Krevvata M, et al. Inhibition of leukemia cell engraftment and disease progression in mice by osteoblasts. Blood. 2014; 124:2834-46.). Osteoblast numbers were maintained by treating leukemic mice with a regimen of intermittent parathyroid hormone (PTH), which increases osteoblast numbers (Jilka R L, et al., Increased bone formation by prevention of osteoblast apoptosis with parathyroid hormone J Clin Invest. 1999; 104:439-46) without affecting serotonin signaling. To preserve the integrity of the BM microenvironment and the hematopoietic system, dsRed-MLL/AF9-induced blasts from leukemic mice were injected into non-irradiated wild type (WT) recipient mice. PTH failed to curtail leukemia growth, as neither disease progression, nor lifespan (FIG. 1A) were affected in PTH-versus vehicle-treated mice. Moreover, PTH did not affect serotonin signaling since expression of Cyclins D1, D2 and E1 (targets suppressed by serotonin-HTR1B signaling (Yadav V K, et al. Lrp5 Controls Bone Formation by Inhibiting Serotonin Synthesis in the Duodenum. Cell. Elsevier Inc; 2008; 135:825-37.)) did not change in the bones of PTH-versus vehicle-treated mice. These results suggested that engagement of a specific pathway dependent on serotonin receptor signaling may mediate the protective effect of osteoblasts against AML progression.

### Example 20: Ablation of Serotonin Receptor 1b (HTR1B) in Osteoblasts Prevents AML Progression

Since our results suggest that the protective effect of osteoblasts against leukemia progression does not rely solely on the number of osteoblasts, but rather on engagement of serotonin receptor signaling, we examined the specific signaling pathway involved. Among the 14 described serotonin receptors, only 3 are expressed in primary osteoblasts: Htr1b, Htr2a, and Htr2b. HTR1B is the main serotonin receptor that controls osteoblasts numbers. We thus analyzed the contribution of HTR1B to leukemia progression through the use of Htr1b−/− mice. Wild-type Htr1b+/+ mice injected with MLL/AF9 consistently developed leukemia and died within 14-19 days following transplantation (FIG. 1B), displaying splenomegaly (FIG. 1B), blast infiltration in BM, liver and spleen as well as peripheral blood neutrophilia, lymphocytopenia and monocytosis. In contrast, 100% of Htr1b−/− littermate mice examined (n=29), remained leukemia-free for at least 90 days after transplantation, the entire time they were observed (FIG. 1B). Upon harvest, all analyzed Htr1b−/− tissues were free of MLL/AF9 cells.

In view of these observations, we asked at what stage during osteoblast differentiation is Htr1b expression necessary for leukemia progression. For this purpose, we inactivated Htr1b either in leptin receptor-expressing (LepR+) mesenchymal stromal cells (MSC) (Zhou B O, et al. Leptin-receptor-expressing mesenchymal stromal cells represent the main source of bone formed by adult bone marrow. Cell Stem Cell. 2014; 15:154-68) or in osteoblasts. We found that ablating Htr1b expression in LepR+MSCs using the LepR-Cre line (32) did not hinder leukemia progression¬and lethality (FIG. 1C). Next, we inactivated Htr1b in cells fully committed to the osteoblast fate using the collagen type-I, alpha-1 (Col1a1)-Cre line (Dacquin R, et al., Mouse alpha1(I)-collagen promoter is the best known promoter to drive efficient Cre recombinase expression in osteoblast. Developmental Cell. 2002; 224:245-51.) (Htr1b c-osb−/−, FIG. 1D) or in differentiated osteoblasts, using the osteocalcin (OCN)-Cre line (34) (Htr1b d-osb−/−, FIG. 1E). In both scenarios, we observed a marked reduction in leukemia progression) and either reduced mortality by 70% in Htr1b c-osb−/− (FIG. 1D), or complete prevention of lethality in Htr1b d-osb−/− mice (FIG. 1E) injected with MLL/AF9 cells, for the entire time that they were observed. In contrast, all WT control mice died within 14-17 days following MLL/AF9 transplantation. Recombination efficiency was twice as effective using OCN-Cre than Col1a-Cre at the Htr1bflfl1 locus, potentially explaining the difference in the level of protection against leukemia between the two conditional models. These data show that ablation of Htr1b in committed osteoblasts is sufficient to confer a close to complete protection against AML and to increase survival.

To determine whether Htr1b deletion in bone can limit AML progression after engraftment, we inducibly-inactivated Htr1b following AML transplantation using the tetracycline-dependent Tg (Sp7-tTA,tetO-EGFP/cre) 1 Amc/J (Osx-Cre) line (Rodda S J, et al., Distinct roles for Hedgehog and canonical Wnt signaling in specification, differentiation and maintenance of osteoblast progenitors. Development. Oxford University Press for The Company of Biologists Limited; 2006; 133:3231-44.), which in adult mice deletes genes in cells at every stage of the osteoblast differentiation pathway. Delaying Osx-Cre expression until postnatally restricts deletion to committed osteoblasts (Mizoguchi T, et al. Osterix marks distinct waves of primitive and definitive stromal progenitors during bone marrow development. Developmental Cell. 2014; 29:340-9.), therefore, Htr1b fl/fl; Osx-Cre mice were born, weaned and kept on doxycycline (DOX) containing diet to suppress transgene activation. DOX removal after MLL/AF9 injection in Htr1b fl/fl; Osx-Cre mice increased survival (FIG. 1F) and decreased leukemia burden (FIG. 1G). Moreover, two mice showed complete protection against leukemia and survived the entire period of observation (FIG. 1F). Detailed analysis of their leukemia burden showed increasing signal up to day 12 after transplantation followed by a steady decrease to basal levels which signifies complete clearance from AML (Supplementary FIG. 1SL). These results suggest that activation of pathways channeled through Htr1b in osteoblasts by AML cells, is a prerequisite to allow leukemia growth in the BM. Moreover, inhibition of HTR1B signaling in osteoblasts post AML engraftment can limit—and at cases clear—the disease, improving leukemia burden and survival.

To address if the partial rescue observed was due to the limited decrease in serum 5-HT levels, we examined whether the selective Htr1b receptor antagonist SB224289 (SB9) (Gaster L M, et al. The selective 5-HT1B receptor inverse agonist 1′-methyl-5-[[2′-methyl-4“-(5-methyl-1,2,4-oxadiazol-3-yl) biphenyl-4-yl]carbonyl]-2,3,6,7-tetrahydro-spiro[furo[2,3-f]indole-3,4-”piperidine] (SB-224289) potently blocks terminal 5-HT autoreceptor function both in vitro and in vivo. J Med Chem. 1998; 41:1218-35.) could confer a protective effect of a magnitude similar to that observed upon inactivation of Htr1b in osteoblasts. However, as seen following pharmacological inhibition of 5-HT synthesis, SB9 only partially-protected MLL/AF9-injected mice (FIG. 1H). Although SB9-treated mice injected with MLL/AF9 showed a significant increase in survival as compared to vehicle treated ones (FIG. 1H), they eventually developed leukemia and died. Importantly, the administered SB9 dose was effective in abolishing 5-HT binding to HTR1B. SB9 successfully inhibited 5-HT signaling since expression of Cyclins D1, D2 and E1 (suppressed upon 5-HT signaling through HTR1B in bone (Yadav V K, et al. Lrp5 Controls Bone Formation by Inhibiting Serotonin Synthesis in the Duodenum. Cell. Elsevier Inc; 2008; 135:825-37.)) was upregulated in the bone of SB9-treated mice. As a control, expression of Col1a1, an osteoblast-specific gene, was not affected by SB9 treatment. Therefore, SB9 treatment efficiently antagonized 5-HT signaling. The partial rescue from AML progression by either inhibition of 5-HT synthesis=or signaling (SB9) as compared to the close to complete protection seen after genetic Htr1b ablation, suggested that the main pro-leukemic effect of HTR1B may be mediated through a ligand different than serotonin.

FIGS. 1A-1H show the results of ablation of serotonin receptor 1b (Htr1b) in osteoblasts prevents AML progression. (A) Survival curve of wild-type (WT) mice treated with vehicle (n=4) or parathyroid hormone (PTH, n=7) and injected with MLL/AF9 AML cells. (B-E) Survival curves of WT MLL/AF9-injected mice, their spleen weights and representative epifluorescence images (radiance p/sec/cm2/sr) of leukemia progression 14 days after MLL/AF9 injection in: (B) Htr1b^{-/-} (n=29) and Htr1b^{+/+} littermates (n=13); (C) Htr1b^{fl/fl}, LepR-Cre: Htr1b_{Lep-R}^{-/-} (n=8) and Htr1b_{Lep-R}^{+/+} littermates (n=6); (D) Htr1b^{fl/fl}; Col1a1-Cre: Htr1b_{c-osb}^{-/-} (n=11) and Htr1b_{c-osb}^{+/+} littermates (n=12)—the 4 Htr1b_{c-osb}^{-/-} mice that developed leukemia are represented with red stars in the histogram of spleen weight and excluded from the statistical analysis—; (E) Htr1b^{fl/fl}; OCN-Cre: Htr1b_{d-osb}^{-/-} (n=5) and Htr1b_{d-osb}^{+/+} littermates (n=10). Orange arrow indicates the systematic genetic interrogation approach followed. (F) Survival curve of Htr1b^{fl/fl}; Osx-Cre: Htr1b_{Osx}^{+/+} (doxycycline-DOX-removed 24 h after MLL/AF9 injection; n=9) and Htr1b_{Osx}^{+/+} (kept on DOX, n=6). (G) Leukemia burden quantification (total flux, photons/sec) at day 12 after MLL/AF9 injection, Htr1b_{Osx}^{-/-} (DOX, n=6), Htr1b_{Osx}^{-/-} (no-DOX; n=9). (H) Survival curve of WT mice injected with MLL/AF9 cells and treated with either vehicle (n=10) or the HTR1B antagonist SB224289 (SB9) (n=10). All survival curves shown are Kaplan-Meier curves with the p-value of log rank (Mantel-Cox) test between the indicated groups. All data are represented as mean±SEM, statistical analysis done with unpaired t-test.

### Example 21: AML Cells Preferentially Convert Tryptophan into Kynurenine

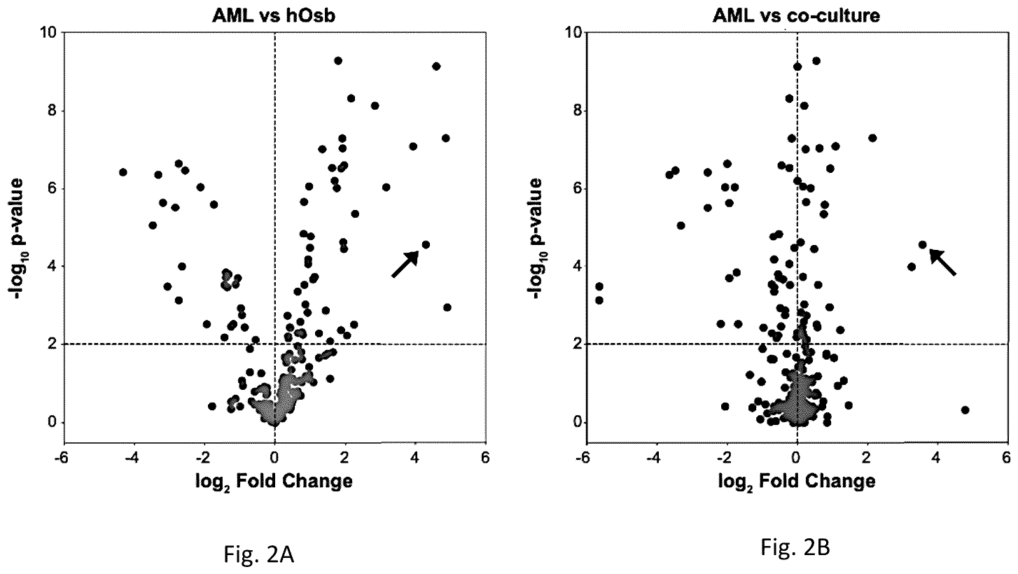

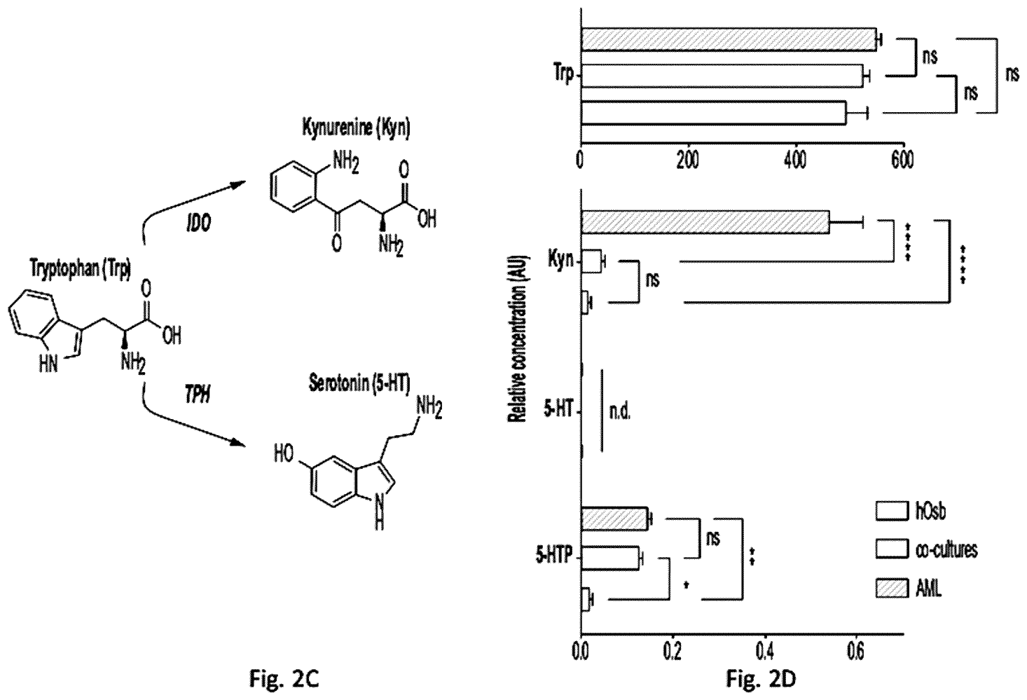

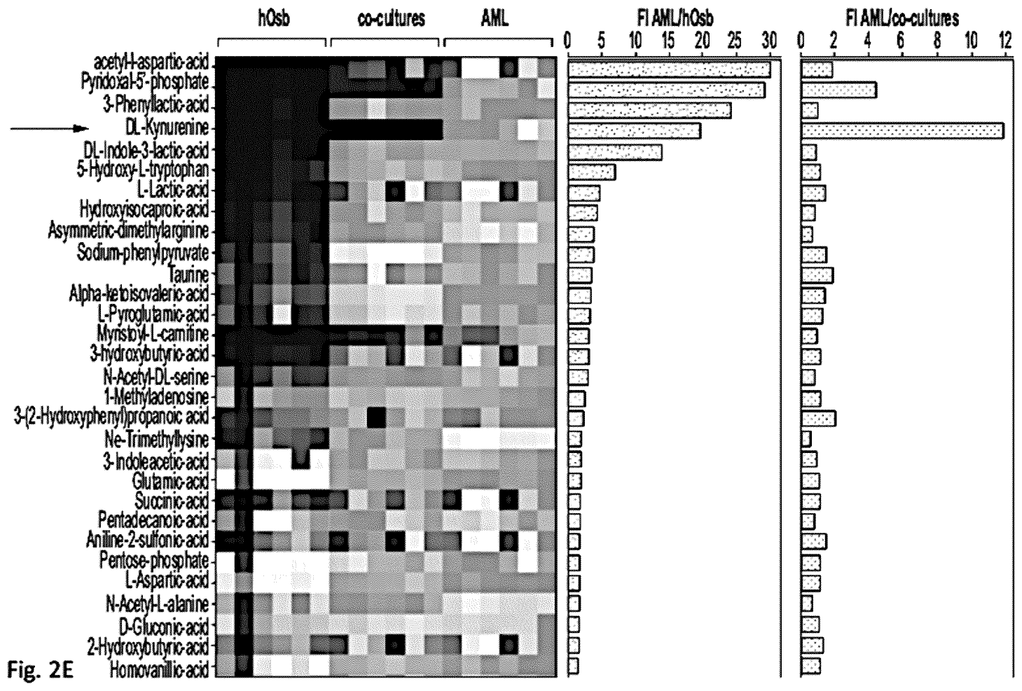

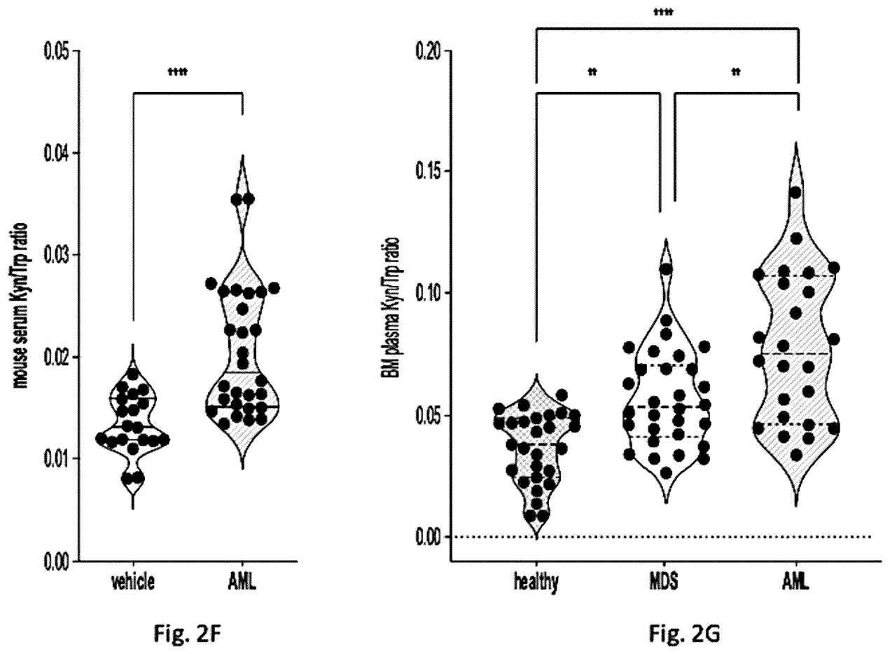

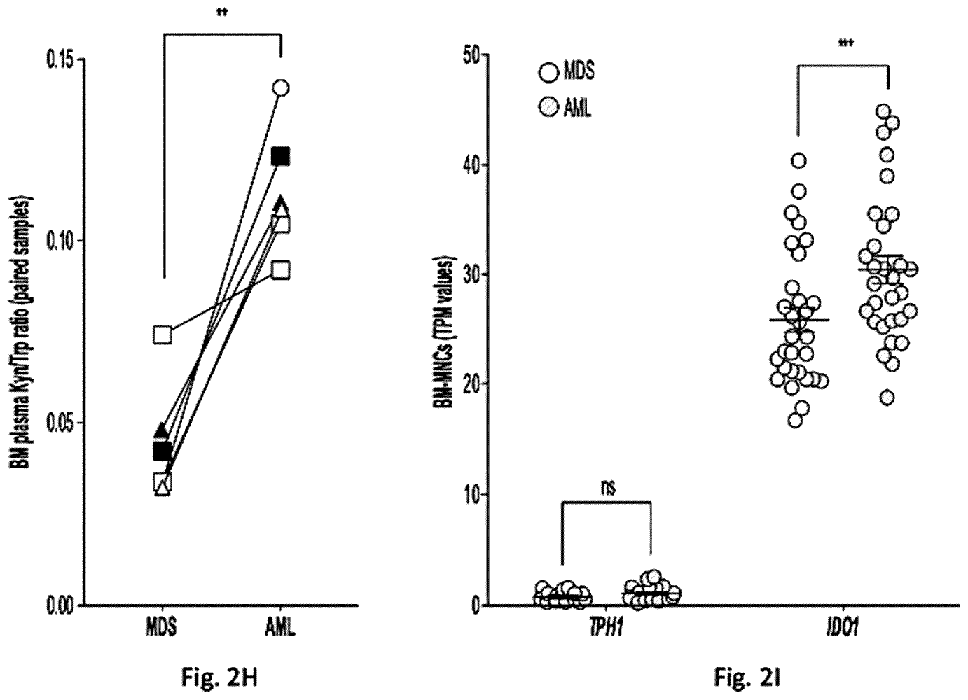

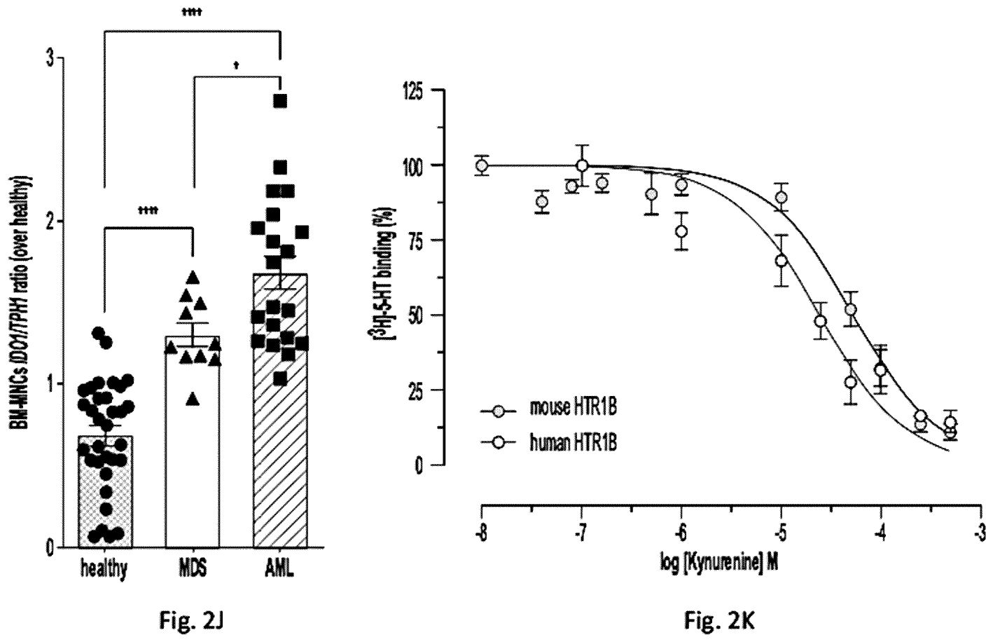

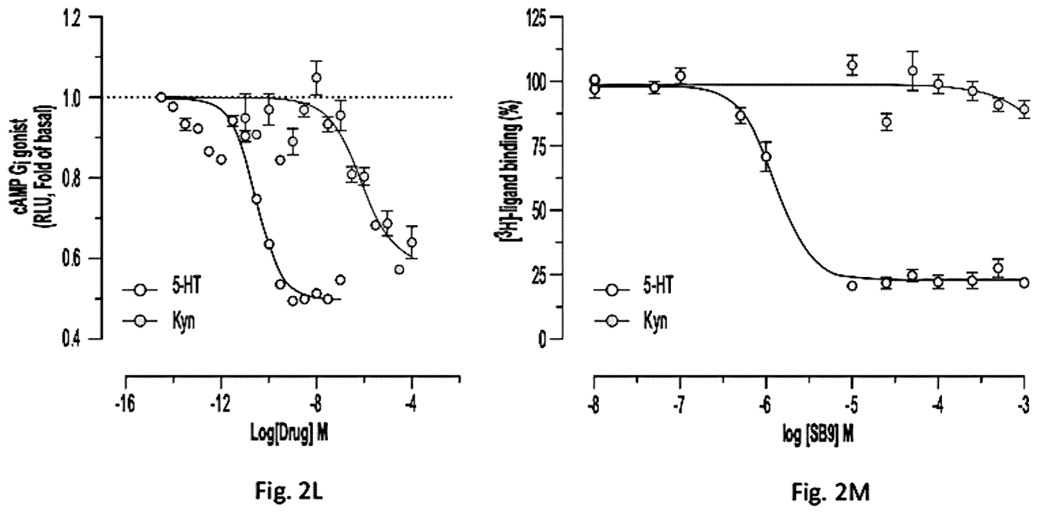

To examine in a disease-relevant approach whether AML cells engage HTR1B in osteoblasts through a ligand different from serotonin, we leveraged an in vitro system using primary human osteoblasts from healthy individuals co-cultured with a human AML cell-line (OCI-AML3). To assess the contribution of secreted soluble factors that may act as HTR1B putative ligands, untargeted metabolomic profiling was performed on supernatants from either cell type alone or in co-culture, using a panel of 466 metabolites. We focused on those with coefficient of variation (CV) below 30% and integrated the data to identify metabolites showing a stronger combination of fold change and statistical significance. Our strategy was to first identify metabolites highly secreted by AML cells and not by osteoblasts (FIG. 2A), and then to select those displaying significant changes in their levels following co-culture (FIG. 2B). This two-step analysis pinpointed one metabolite: kynurenine (Kyn), the levels of which were not only increased by 20-fold in supernatants from AML cells as compared to osteoblasts (see arrow in FIG. 2A) but at the same time, was the metabolite whose secretion by AML cells was most decreased after co-culture with osteoblasts (see arrow in FIG. 2B). Kyn, like serotonin (5-hydroxytryptamine, 5-HT), is a major tryptophan (Trp) catabolite. While the ubiquitous indoleamine 2,3-dioxygenases (IDO1/IDO2) or the hepatic tryptophan 2,3-dioxygenase (TDO) enzymes catalyze conversion of Trp into Kyn, tryptophan hydroxylase-1 (TPH1) catalyzes the production of duodenal serotonin also from Trp (FIG. 2C). Trp levels were similar among all the supernatants analyzed (FIG. 2D). Interestingly, 5-HT levels were below the limits of detection, and the levels of the 5-HT metabolite 5-hydroxytryptophan (5-HTP) were not altered in co-culture supernatants (FIG. 2D). These observations were further validated by liquid chromatography-mass spectrometry (LC-MS) targeted assays.

A stringent analysis focusing on the metabolites with CV<15%, revealed that similar to Kyn, pyridoxal-5′-phosphate (PLP, the active form of Vitamin B6) was increased 29-fold in supernatants from AML cells as compared to osteoblasts (FIG. 2E, grey histogram) and after Kyn, was the second most highly decreased metabolite upon co-culture of AML with osteoblasts (FIG. 2E-blue histogram). PLP is a necessary cofactor for more than 160 enzymes-reviewed in (Percudani R, et al., A genomic overview of pyridoxal-phosphate-dependent enzymes. EMBO Rep. 2003; 4:850-4-, including several ones in the Kyn pathway, suggesting that its downregulation may be another means of Kyn depletion in the presence of osteoblasts.

### Example 22: High Kynurenine Levels are a Hallmark of MDS and AML

To determine the in vivo significance of Kyn in AML, we measured circulating Kyn and Trp levels in leukemic mice and confirmed that the Kyn-to-Trp ratio (an indicator of IDO1 activity) was elevated in the peripheral blood serum of mice injected with MLL/AF9 cells as compared to control, vehicle-injected mice (FIG. 2F). To assess if our findings in vitro and in murine models were recapitulated in human leukemia, specifically within the BM niche compartment, we examined whether induction of Kyn secretion is a broad feature of AML or the pre-leukemic myelodysplastic syndrome (MDS) patients. We found that the Kyn/Trp ratio within the BM plasma of MDS and AML patients was significantly higher than in aged-matched healthy controls (FIG. 2G). Moreover, we compared Kyn/Trp ratio levels within the BM plasma of paired MDS and progressed-to-AML patient samples: in the 6 paired samples analyzed, Kyn/Trp ratio levels were increased in the BM plasma at the AML stage as compared with their MDS stage sample (FIG. 2H), suggesting that increased Kyn production correlates with disease progression.

RNAseq analysis of BM mononuclear cells (BM-MNCs) from MDS and AML patients showed that whereas TPH1 expression is very low (0.74±0.06 in MDS and 1.09±0.11 in AML, transcript per million-TPM-), expression of IDO1 is much higher (25.89±1.12 MDS and 30.48±1.22 AML) (FIG. 2I). Quantitative PCR analysis of BM-MNCs of additional independent cohorts of healthy subjects, MDS and AML patients, identified a similar progressive increase in the IDO1/TPH1 ratio from healthy controls as compared to patients. Moreover, this increase was similarly observed along the progression of disease severity from MDS to AML (FIG. 2J).

Table 1 shows the clinical characteristics and TPM values of AML and MDS patients used for RNAseq data. Table 1 is related to FIG. 2I.

Collectively, these results identify kynurenine as an oncometabolite, demonstrating preferential catabolism of Trp towards the Kyn pathway in cells of MDS and AML patients, as well as increased levels of the metabolite in their BM plasma. A progressive increase in Kyn production appears to occur as the disease pathogenesis proceeds from MDS to AML.

### Example 23: Kynurenine Binds to and Regulates HTR1B Signaling

The increased and preferential production of Kyn over 5-HT by leukemic cells, together with the partial protective effect caused by the HTR1B antagonist SB9 prompted us to examine whether Kyn could be a previously unappreciated ligand of HTR1B. To address whether Kyn is a serotonin receptor ligand, we performed competition binding and functional assays on HEK293T cells overexpressing mouse or human HTR1B. Kyn was able to compete the binding of 25 nM [3H]-5-HT to mouse (IC50 of ˜54 μM) and human (IC50 of ˜24 μM) HTR1B (FIG. 2K and Table 2) in membranes of HEK293T cells overexpressing the murine or human receptor respectively; as a control, 5-HT showed similar competitive binding activity for both receptors (Table 2). Similarly, Kyn competed the binding of the potent serotonin receptor antagonist [3H]-GR125743 to HTR1B with a Ki of ˜17 μM (Table 2) in membranes isolated from HEK293T cells stably overexpressing the human HTR1B. Moreover, and in agreement with its binding properties, Kyn acts as a partial agonist of Gi/o-mediated cAMP production through HTR1B with an EC50 of ˜772 nM (FIG. 2L and Table 2).

Since SB9 used to displace 5-HT binding to HTR1B was not able to effectively hinder AML in vivo (FIG. 1H), we examined whether it could displace Kyn binding to HTR1B. However, at a concentration equal to the one administered in vivo (˜90 μM, FIG. 1H), SB9 had no effect on Kyn binding to the murine HTR1B receptor (FIG. 2M). Altogether, these experiments demonstrate that Kyn is a partial agonist of HTR1B both in mouse and human, able to regulate its signaling.

FIG. 2. Kynurenine is an oncometabolite increased in the BM niche of MDS and AML patients that binds to HTR1B. (A-B) Volcano plots for metabolites with coefficient of variation (CV)<30% comparing OCI-AML3 cells untreated (AML) and human osteoblasts (hOsb) (A) or AML cells untreated versus co-cultures (24 h) (B), arrows point to kynurenine. (C) Trp catabolismscheme. (D) Relative abundance of tryptophan (Trp) and its catabolic metabolites: kynurenine (Kyn), serotonin (5-HT) and 5-hydroxytryptophan (5-HTP) in the indicated supernatants at 24 h (n=6); two-way ANOVA. (E) Heat-map of the first 30 metabolites with CV <15% and histograms of fold-change of AML vs. hOsb (grey) or AML vs. co-culture (blue). (F) Violin plots of Kyn/Trp ratio levels in serum circulating levels of control-(n=19) vs. MLL/AF9-injected (n=28) mice; unpaired-t test. (G) Violin plots of Kyn/Trp ratio levels in bone marrow (BM) plasma from healthy (n=27), MDS (n=30) and AML (n=24) patients; one-way ANOVA. (H) Kyn/Trp levels in paired BM plasma samples at MDS stage and its corresponding transformed-AML stage (n=6); paired t-test. (I) RNAseq analysis of BM mononuclear cells (BM-MNCs) from MDS (n=30) and AML (n=30) patients (transcript-per-million-TPM-) for TPH1 and IDO1; two-way ANOVA. (J) IDO1/TPH1 mRNA ratio in BM-MNCs from healthy (n=32), MDS (n=10) and AML (n=20) patients; one-way ANOVA. (K) Concentration dependence of the Kyn-mediated competition of [³H]-5-HT (25 nM, 41.3Ci/mmol) binding by HEK293T membranes overexpressing the mouse (n=4 experiments) or the human receptor (n=2 experiments), yielding an IC₅₀ of 54.1 μM and 24.4 μM respectively (see Table 2 for details). (L) G_{i/o}-mediated cAMP inhibition assays (n=14). (M) Binding of [³H]-5-HT (25 nM, 41.3 Ci/mmol) or [³H]-Kyn (50 μM, 0.125 Ci/mmol) was measured with Htr1b-overexpressing-HEK293T membranes in the presence of increasing concentrations of SB9 (n=4). Non-linear regression fitting was used to fit the isotherms, and the best-fit values and statistics of the fit are shown in Table 2. All data are expressed as mean±SEM. See also Table 2.

### Example 24: Kynurenine Binds to and Regulates HTR1B Signaling

To explore in vivo the significance of Kyn for leukemia progression, we inhibited its synthesis by suppressing IDO activity in mouse and human AML cells. We used a CRISPR-Cas9 editing strategy designing a series of different single-guide RNAs (sgRNAs) targeting Ido1 exons 3 or 4, which encode critical portions of the enzyme catalytic site and are common to all IDO isoforms.

First, Ido1 was genetically ablated in the myelomonocytic leukemia cell line WEHI-3B. High deletion efficiencies were achieved on WEHI-3B cells, especially when combining two sgRNAs targeting exon 3. Mice receiving the Cas9-only WEHI-3B control cells died within 2.5 weeks after injection, while the ones injected with gRNA #146 (SEQ ID 1) alone or in combination with gRNA #196 (SEQ ID 22) showed significant increased survivals. Importantly, the decrease in Kyn levels as well as, the protective effect of Ido1 deletion were proportional to the efficiency of Ido1 deletion.

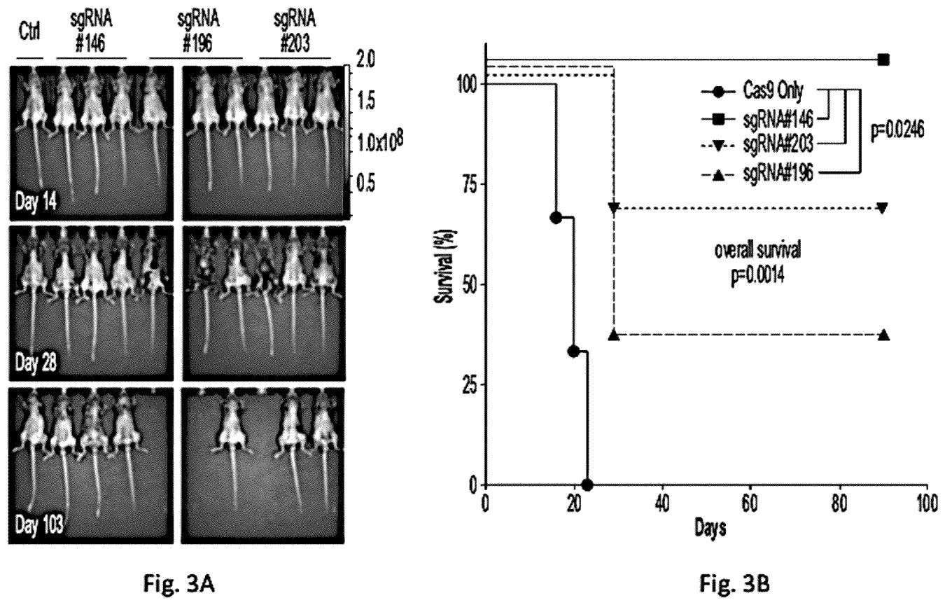

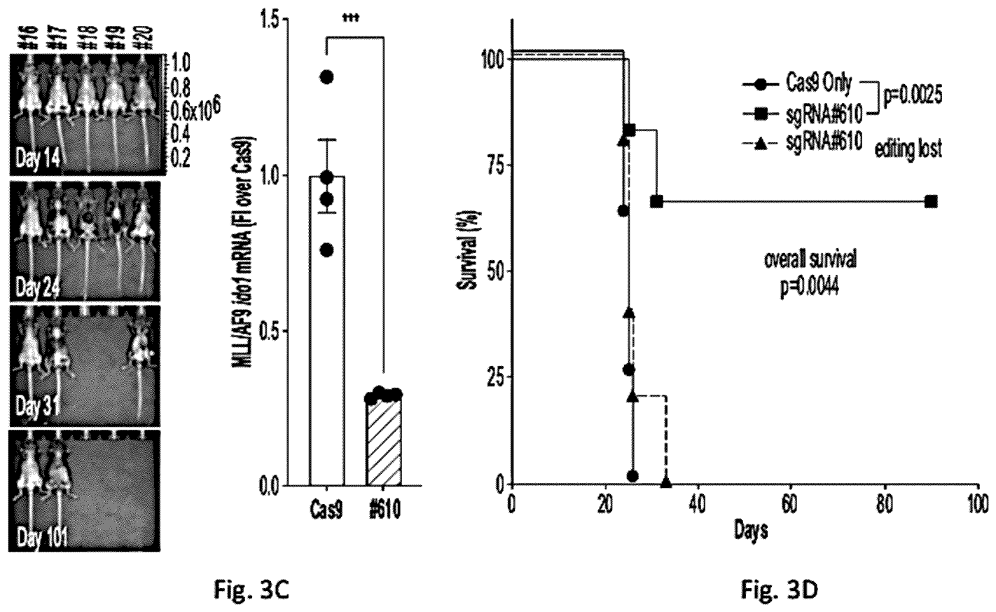

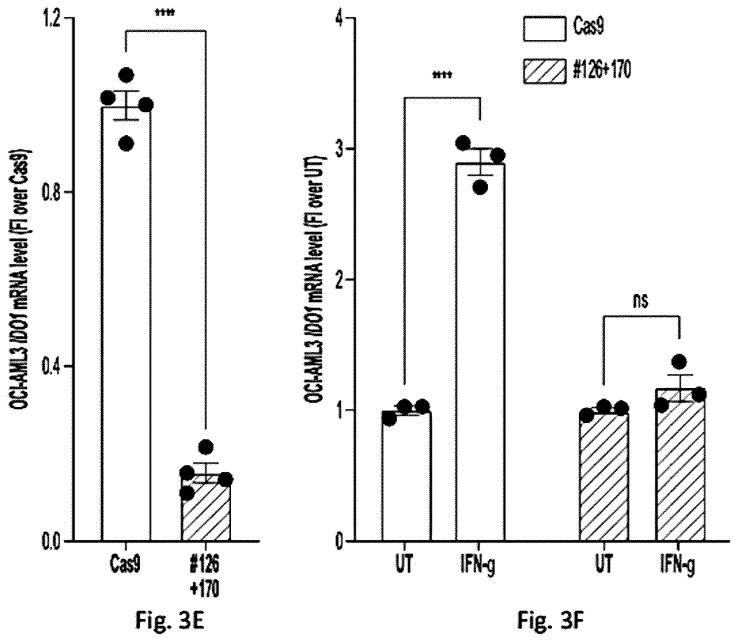

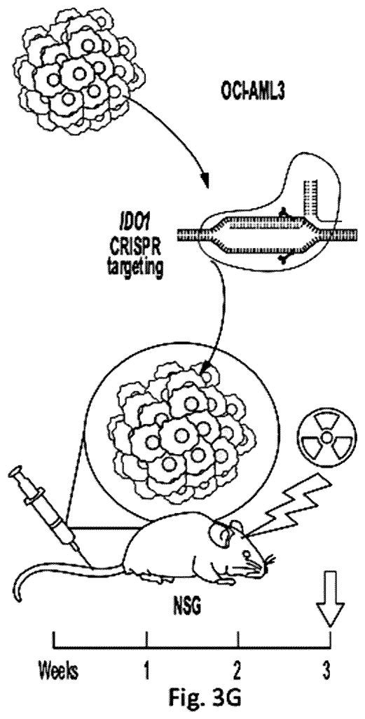

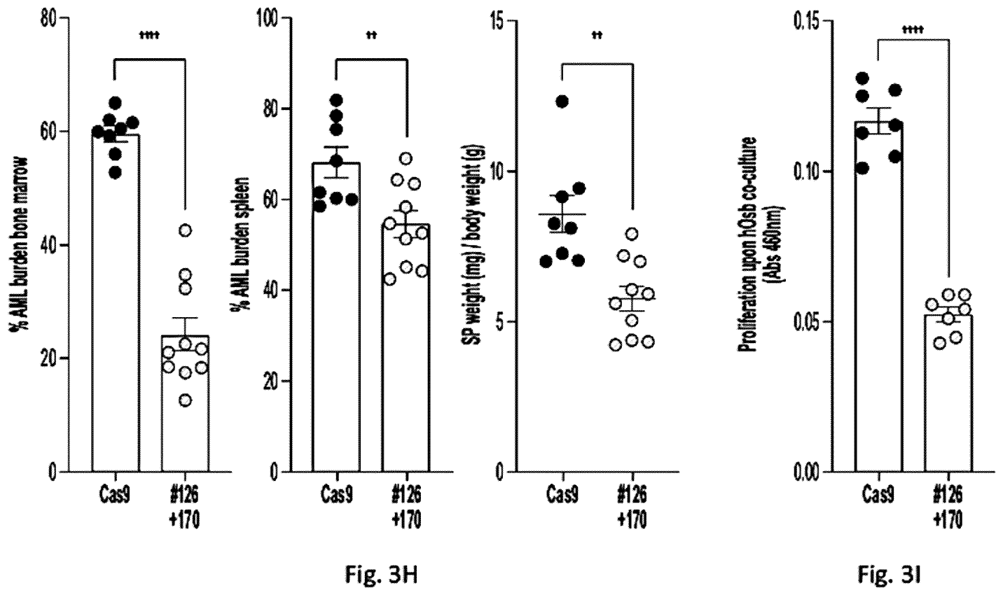

Next, we used sgRNAs targeting Ido1 exons 3 or 4 to modify primary murine leukemia cells. Ido1 exon-3-edited MLL/AF9 cells were transplanted into WT non-irradiated recipients and leukemia progression was monitored (FIG. 3A. While all mice receiving the Cas9-only MLL/AF9 control cells died within 3 weeks after injection (FIG. 3B), Ido1 deletion significantly attenuated (sgRNA #203 (SEQ ID 41) and sgRNA #196 (SEQ ID 22) ˜40% deletion efficiency) or even abrogated (sgRNA #146 (SEQ ID 1) ˜56% deletion efficiency) disease progression, and decreased serum Kyn levels, extending overall survival (FIG. 3B).

CRISPR-Cas9-mediated Ido1 targeting of exon 4 achieved a 70% loss of expression of Ido1 at the mRNA level (FIG. 3C). Injection of Ido1-sgRNA #610 (SEQ ID 61)-edited MLL/AF9 cells into WT non-irradiated recipients led to a significant increase in survival (FIG. 3D). 36% of the mice receiving the MLL/AF9-edited cells showed complete protection against leukemia progression and survived (FIG. 3D. Notably, spleen weight and serum Kyn levels were proportional to the decrease in IDO1 levels as well as to the survival effects. Because the majority of MLL/AF9 cells (˜70%), were efficiently targeted by sgRNA #610 (SEQ ID 61), we reasoned that the small residual fraction of unedited AML cells would outcompete edited cells over time. Indeed, sequencing analysis of BM cells from moribund mice showed that IDO1 activity was not compromised in 45% of the mice, which exhibited a percentage of unedited (WT) sequence between 60-100%. These results suggested that the few unedited cells present in the initially injected population had a clonal advantage over the Ido1-edited ones and were responsible for disease progression. Only two of the non-rescued mice showed <10% unedited cells; however, in one of them (BM #19), an in-frame deletion may have preserved IDO1 functionality, allowing AML to progress, whereas in the other one (BM #12), a disrupted IDO1 frameshift might explain its prolonged survival.

The relevance of IDO1 in the progression of human leukemia was tested using the OCI-AML3 AML cell line. OCI-AML3 cells nucleofected with Cas9 and the combination of sgRNAs #126 (SEQ ID 82) and #170 (SEQ ID 103) (targeting exon 3 of IDO1) showed high deletion efficiency (˜85%, FIG. 3E) and when exposed to IFN-γ-a strong inducer of IDO1-CRISPR-Cas9 targeted OCI-AML3 cells failed to upregulate its expression (FIG. 3F). Transplantation of OCI-AML3 IDO1-targeted cells into sub-lethally irradiated NOD.Cg-Prkdcscid Il2rgtm1Wjl/SzJ (NSG) mice (FIG. 3G), resulted in delayed disease progression as seen by a ˜60% decrease in BM AML burden, ˜20% decrease in spleen AML burden, and significantly reduced spleen weights (FIG. 3H). Consistent with the decreased AML burden, the level of IDO1 expression in BM of NSG mice at harvest showed a 73% decrease compared with control (Cas9 only) injected mice confirming that, a possible outgrowth of a reversion mutant was residual. Additionally, serum Kyn levels were reduced by ˜30%. Of note, although OCI-AML3 IDO1-targeted cells did not show any intrinsic proliferative defect as compared to the control (Cas9 only) ones, their proliferation was decreased when placed in co-cultures with primary human osteoblasts (FIG. 3I). In contrast, simulating the IDO1 upregulation triggered by osteoblasts, overexpression of IDO1 in OCI-AML3 cells promoted their proliferation in a dose-dependent manner.

Taken together, these results demonstrate that IDO1 is required to sustain AML cell proliferation in an osteoblast-dependent manner, and that genetic ablation of IDO1 suppresses AML growth in a dose-dependent manner, suggesting that disease severity is inversely correlated to the expression of Ido1.

FIG. 3. Genetic inhibition of kynurenine production hinders AML progression. (A) Representative epifluorescence images of leukemia progression in WT mice injected with MLL/AF9-CRISPR/Cas9-edited cells (sgRNAs: #146 (SEQ ID 1), #196 (SEQ ID 22) and #203 (SEQ ID 41)) (Ctrl: no leukemia). (B) Survival curve of mice injected with the indicated sgRNAs MLL/AF9-edited or Cas9-only-MLL/AF9 control cells (n=3 all groups). (C) Representative epifluorescence images of leukemia progression in WT mice injected with MLL/AF9-CRISPR/Cas9-edited cells (sgRNAs: #610) (SEQ ID 61) and Ido1 mRNA levels of MLL/AF9-sgRNA #610 (SEQ ID 61)-edited cells before injection (n=4); unpaired t-test. (D) Survival curve of WT mice injected with MLL/AF9-sgRNA #610 (SEQ ID 61)-edited cells (n=11) or Cas9 only control (n=9). Mice showing >60% of unedited (WT) sequence in their BM after harvesting are depicted as sgRNA #610_{editing lost} (green; n=5) (SEQ ID 61). (E) IDO1 mRNA levels in OCI-AML3 cells nucleofected with Cas9 and sgRN #610 (SEQ ID 61) used in transplant experiment. (F) IDO1 mRNA levels in OCI-AML3 cells exposed to IFN-γ (overnight, 50 ng/ml, n=3); two-way ANOVA. (G) Outline of transplantation assay with OCI-AML3 CRISPR/Cas9-IDO1-targeted cells in NSG mice. (H) AML burden in bone marrow, spleen, and spleen weight (mg)—referred to total body weight (g)—of NSG mice 3 weeks after injection of OCI-AML3 cells (n=8 Cas9; n=10 #126+170). (I) Proliferation of OCI-AML3 cells upon 72 h of co-culture with primary human osteoblasts (n=7). Survival curves are Kaplan-Meier with p-value of log rank (Mantel-Cox) test between the indicated groups. All data are expressed as mean±SEM. Statistical analysis done with unpaired t-test unless otherwise stated.

### Example 25: AML Cells Induce a Self-Reinforcing Osteoblastic Niche Through SAA1-Mediated IDO1 Upregulation in an HTR1B Dependent Manner

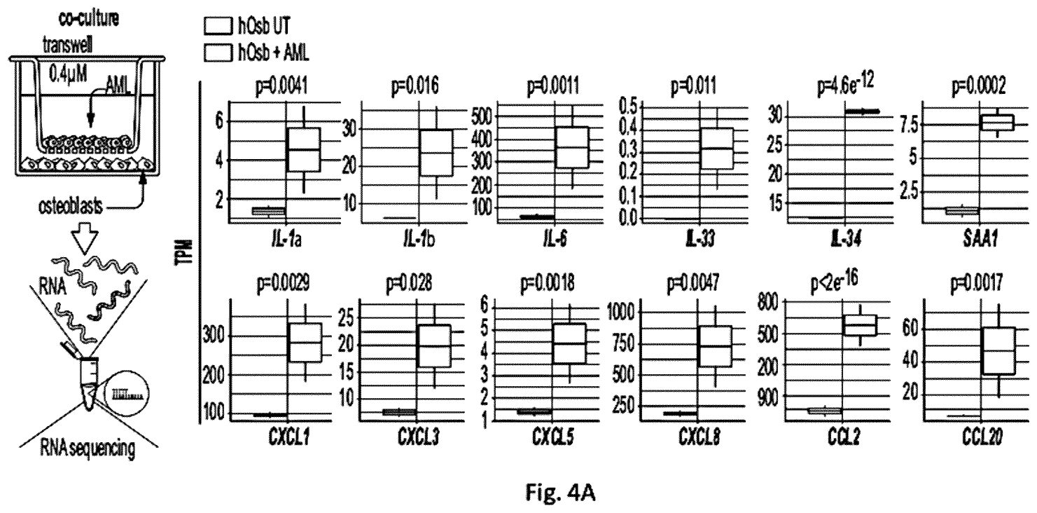

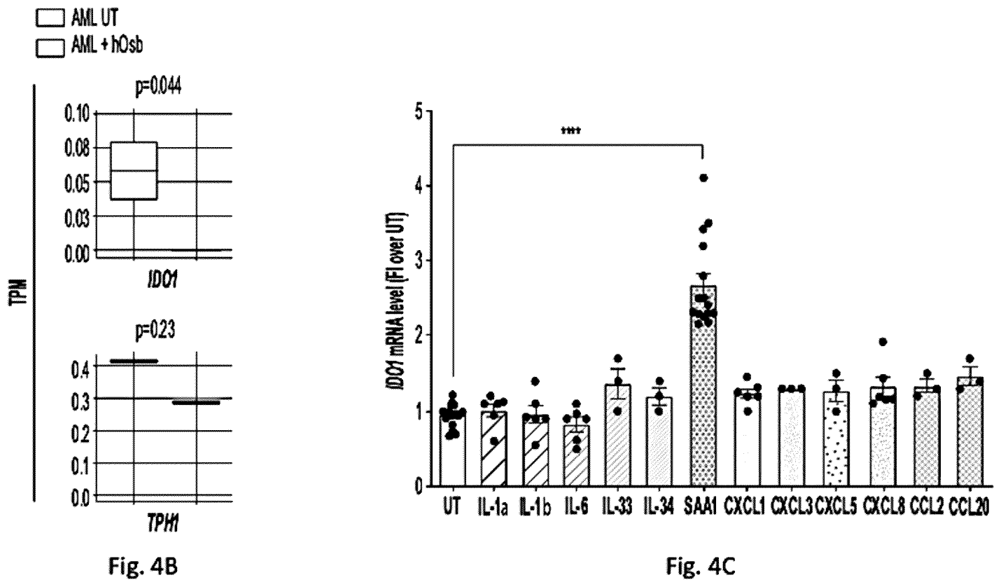

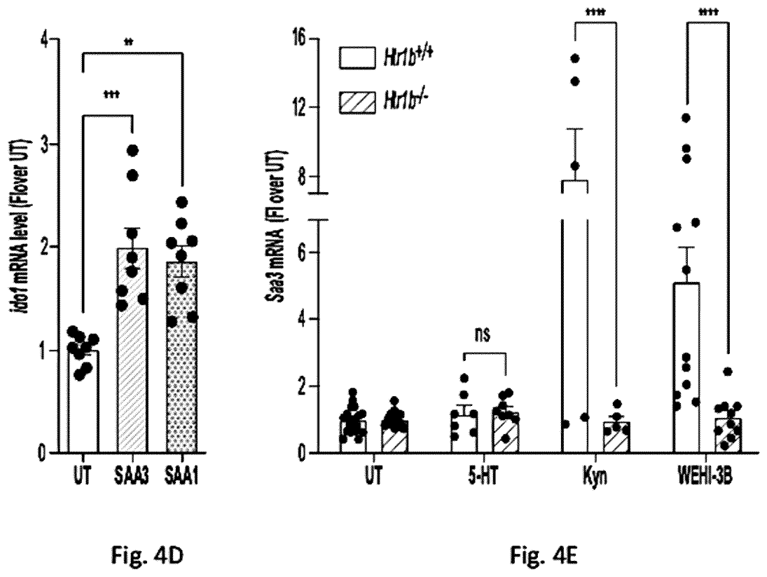

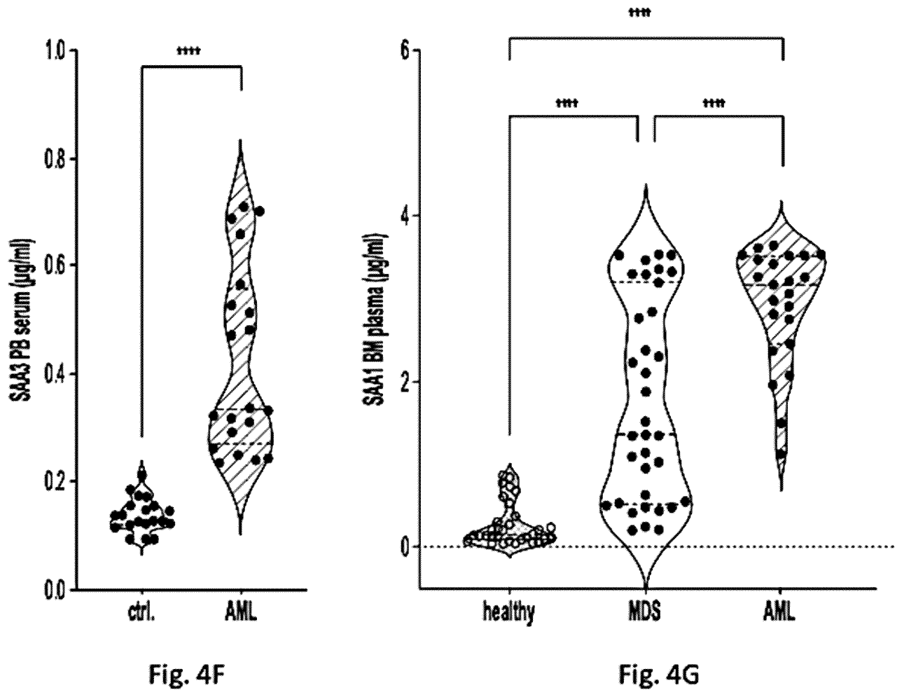

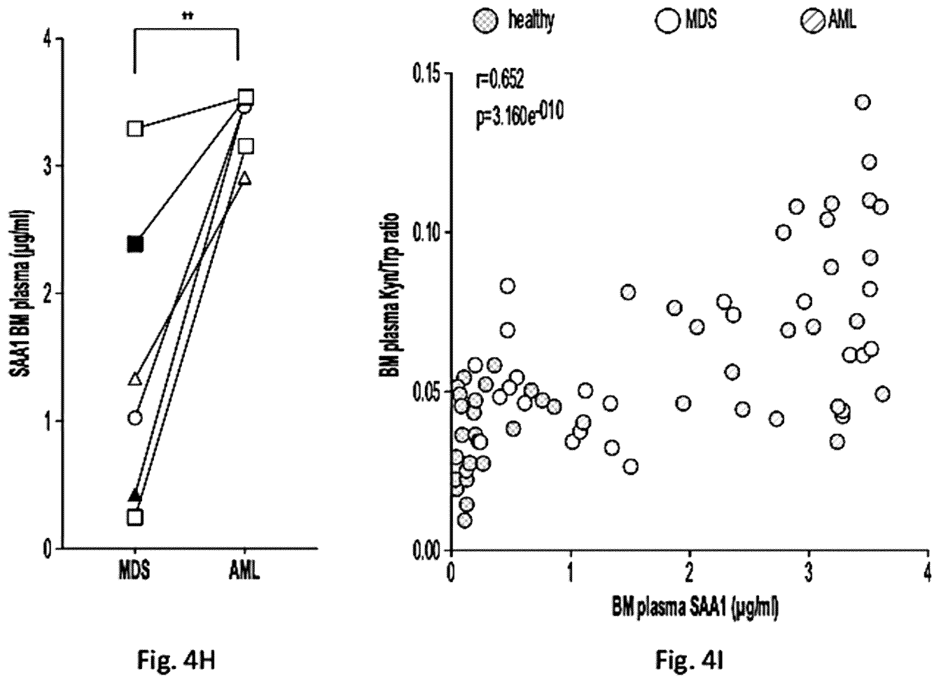

Next, we sought to identify the downstream molecular targets of Kyn in human osteoblasts that render the BM niche permissive to AML engraftment and support proliferation of leukemia cells. For this purpose, and to closely compare our studies in mice and humans, we used the human THP-1 AML cell line, which carries the MLL/AF9 fusion oncogene, the most commonly involved in MLL translocations and a powerful driver of tumor progression. We characterized the transcriptional profile of co-cultures of THP-1 cells with primary human osteoblasts and integrated the data to identify crosstalk signals. RNA sequencing (RNAseq) analysis showed that 137 genes were significantly differentially expressed in osteoblasts exposed to AML cells as compared to osteoblasts cultured alone. Among those, pathway enrichment analysis identified several inflammatory pathways regulating multiple aspects of innate and adaptive immune functions (NF-κB-, TNF- and IL-17-signaling pathways) that were significantly increased in osteoblasts exposed to AML cells. In agreement with these observations, leukemic cells increased NFκB1A expression and induced p65 translocation to the nucleus, in primary osteoblasts isolated from healthy subjects, indicating that AML cells activate canonical NF-κB signaling in osteoblasts. Indeed, gene set enrichment analysis (GSEA) focused on genes encoding secreted-molecules, demonstrated that expression of several pro-inflammatory cytokine and chemokine genes in the NF-κB pathway were highly upregulated in primary human osteoblasts exposed to AML cells (FIG. 4A). This pro-inflammatory signature elicited in osteoblasts by AML cells, was confirmed by qRT-PCR in primary osteoblasts from healthy human subjects co-cultured with the THP-1 or OCI-AML3 AML cell lines. Selected targets were additionally validated through multiplex assessment of protein levels in the corresponding supernatants. Of note, an apoptosis pathway signature was upregulated in osteoblasts exposed to AML cells, and this upregulation correlated with an inflammatory signature in leukemic cells exposed to osteoblasts, suggesting that an inflammation-induced apoptosis pathway maybe the mechanism responsible for bone loss in AML.

More specifically, a parallel RNAseq analysis of the THP-1 AML cells exposed to human primary osteoblasts showed increased expression of IDO1 (log FC 4.6), but no change in TPH1 expression (FIG. 4B). Interestingly, following the initial differential expression analysis, a pathway enrichment analysis highlighted several IDO1-activating pathways. GSEA analysis showed that Trp catabolism as well as the Kyn pathway itself, were upregulated in THP-1 cells exposed to osteoblasts and qRT-PCR analysis confirmed IDO1 upregulation. Notably, genetic ablation of IDO1 by CRISPR/Cas9 editing in OCI-AML3 cells, abrogated the osteoblast-induced upregulation of IDO1 expression observed in the AML cells upon co-culture with primary human osteoblasts. Taken together, these results suggest that AML cells “prime” osteoblasts to secrete factors that stimulate IDO1 expression.

In order to pinpoint these factors, we directly examined whether any of the pro-inflammatory candidate molecules identified to be elicited in primary human osteoblasts by AML cells (FIG. 4A), affected IDO1 expression in the latter. Among them, the rapidly induced acute-phase protein serum amyloid A1 (SAA1) was the only osteoblast-secreted molecule able to upregulate IDO1 expression in OCI-AML3 leukemic cells (FIG. 4C). Most importantly, the ability of SAA1 to upregulate IDO1 expression was observed across several human AML cell lines as well as the MDS-L cell line.

SAA1 is the functional human orthologue of murine Saa3 (41). Similar to SAA1, SAA3 is an acute-phase response protein highly induced during inflammation by IL-1β, TNF-α, and IL-6 through NF-κB signaling (42). Of interest, these cytokines as well as the NF-κB pathway itself, were found to be significantly up-regulated in the RNAseq dataset of human osteoblast exposed to AML cells (FIG. 4A). To assess if our findings in human cells were recapitulated in the mouse model, we examined whether Ido1 upregulation was a general consequence of SAA exposure. We found that, as it is the case in human AML cells exposed to SAA1 (FIG. 4C), recombinant mouse SAA3 upregulated Ido1 expression in murine WEHI-3B AML cells (FIG. 4D). Moreover, recombinant human SAA1 was also able to upregulate Ido1 expression in WEHI-3B cells with a magnitude similar to SAA3 (FIG. 4D), underscoring the notion that this mode of regulation is conserved in mice and humans.

To test whether the AML-elicited SAA response observed in osteoblasts was dependent on Kyn engagement of HTR1B, we used mouse primary osteoblasts isolated from Htr1b−/− or Htr1b+/+ littermate mice. Notably, whereas both Kyn and WEHI-3B AML cells potently upregulated Saa3 expression in mouse osteoblasts, 5-HT had no effect (FIG. 4E). More importantly, both Kyn and WEHI-3B cells failed to upregulate Saa3 expression in Htr1b−/− primary osteoblasts (FIG. 4E). These results demonstrate that Kyn secreted by AML cells upregulates Saa3 expression in osteoblasts in an HTR1B-dependent manner. This upregulation serves as a positive feedback mechanism to amplify Ido1 expression in AML cells.

### Example 26: SAA1 Levels are Elevated in MDS and AML Patients and Correlate with Disease Progression and Kynurenine Levels

To determine the in vivo significance of Saa3 in AML, we measured circulating SAA3 levels in leukemic mice and confirmed that they were elevated in the peripheral blood serum of mice injected with MLL/AF9 cells as compared to control, vehicle-injected mice (FIG. 4F). The relevance of these findings to human disease was assessed by measured SAA1 levels in the BM plasma of MDS and AML patients. In agreement with the increased mRNA expression of SAA1 observed in osteoblasts upon exposure to AML cells (FIG. 4A), BM plasma levels of SAA1 were 6.4- and 10.6-fold times higher in MDS and AML patients—respectively—as compared to aged-matched healthy subjects (FIG. 4G). More importantly, SAA1 concentration in all the paired human samples analyzed was higher in the BM plasma of transformed AML patients versus their paired previous MDS-stage samples (FIG. 4H), suggesting a role of SAA1 in AML pathogenesis. Interestingly, a correlation between Kyn/Trp ratio and SAA1 levels in BM plasma was observed along progression from MDS to AML (FIG. 4I), underscoring a potential prognostic value of the two biomarkers in MDS to AML progression.

FIG. 4. AML cells self-amplify kynurenine production through HTR1B-SAA signaling in osteoblasts. (A) Schematic of RNAseq analysis strategy (left) and box plots (right) of the main secreted molecules significantly upregulated in primary human osteoblasts co-cultured 24 h with the THP-1 AML cell line (n=2); Wald test, two-sided. (B) Box plots for IDO1 and TPH1 from RNAseq analysis of THP-1 cells exposed 24 h to primary human osteoblasts (n=2); Wald test, two-sided. (C) IDO1 mRNA levels in OCI-AML3 cells exposed o/n to the indicated molecules (UT and SAA1 n=15; IL-1α, -1β, -6, CXCL-1 and -8 n=6; IL-33, -34, CXCL-3, -5, CCL-2 and -20 n=3). (D) Ido1 mRNA levels in WEHI-3B cells exposed o/n to recombinant mouse SAA3 or recombinant human SAA1 (n=8). (E) Saa3 mRNA relative level in primary differentiated mouse calvaria from Htr1b^{-/-} and Htr1b^{+/+} littermates, exposed for 24 h to 5-HT (25 nM, n=7-8), Kyn (25 nM, n=5) or the WEHI-3B cell line (n=10-12); two-way ANOVA. (F) Violin plots of SAA3 peripheral blood (PB) serum levels in control (n=20) and MLL/AF9-injected mice (n=20); unpaired t-test. (G) Violin plots of SAA1 BM plasma levels in healthy (n=30), MDS (n=35) and AML (n=23) patients. (H) SAA1 BM plasma levels in paired samples from patients (MDS and corresponding AML-transformed stage) (n=6 paired-samples); paired t-test. (I) Multiple variable data plot of BM plasma levels for SAA1 and Kyn/Trp ratio along healthy, MDS or AML samples; Pearson correlation values are shown for Kyn/Trp ratio and SAA1 BM plasma levels. All data expressed as mean±SEM. Statistical analysis was done with one-way ANOVA unless otherwise stated.

### Example 27: SAA Selectively Promotes Proliferation of AML Cells

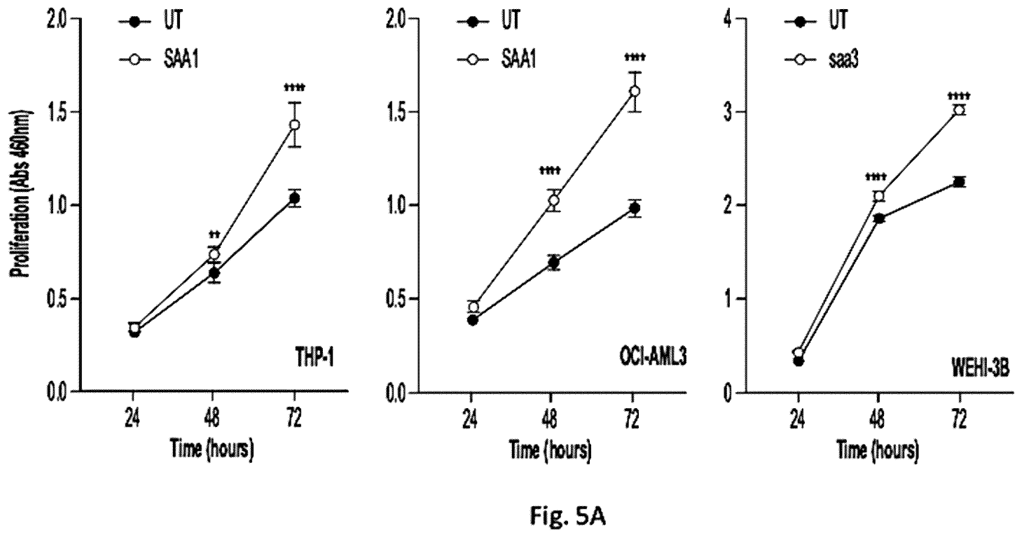

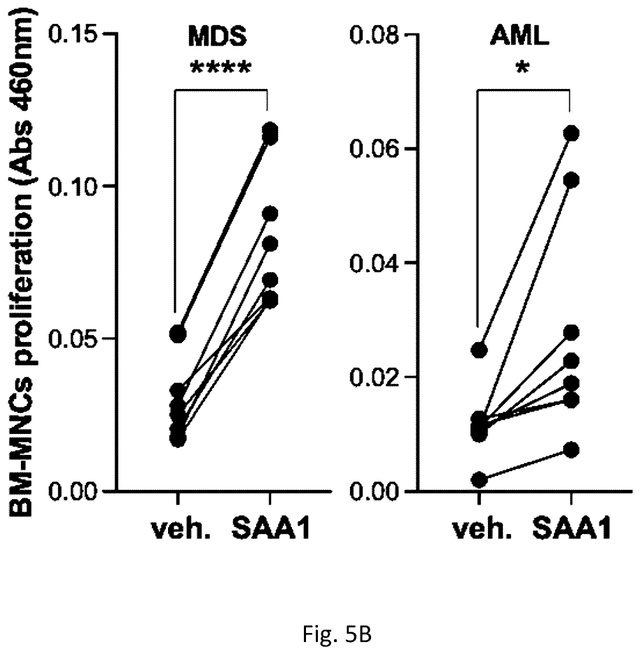

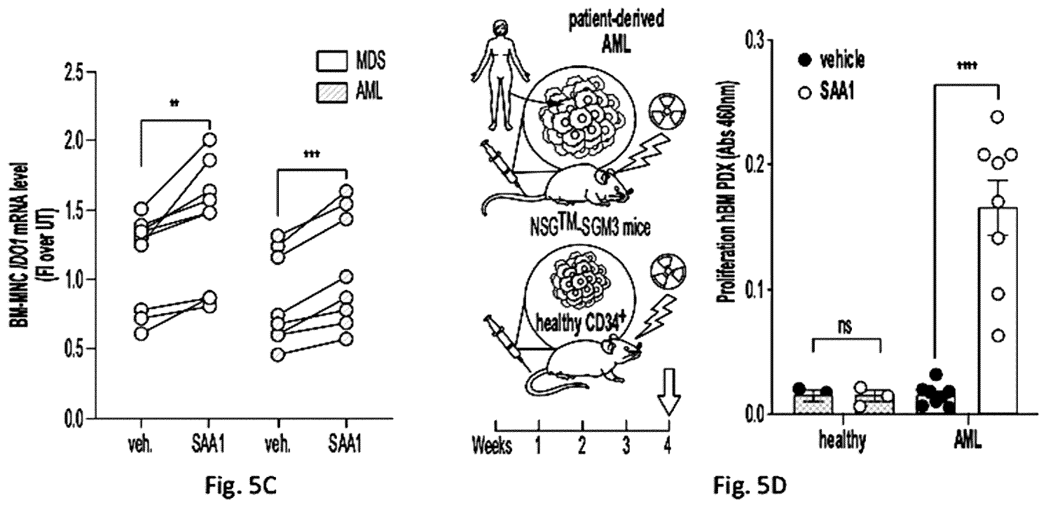

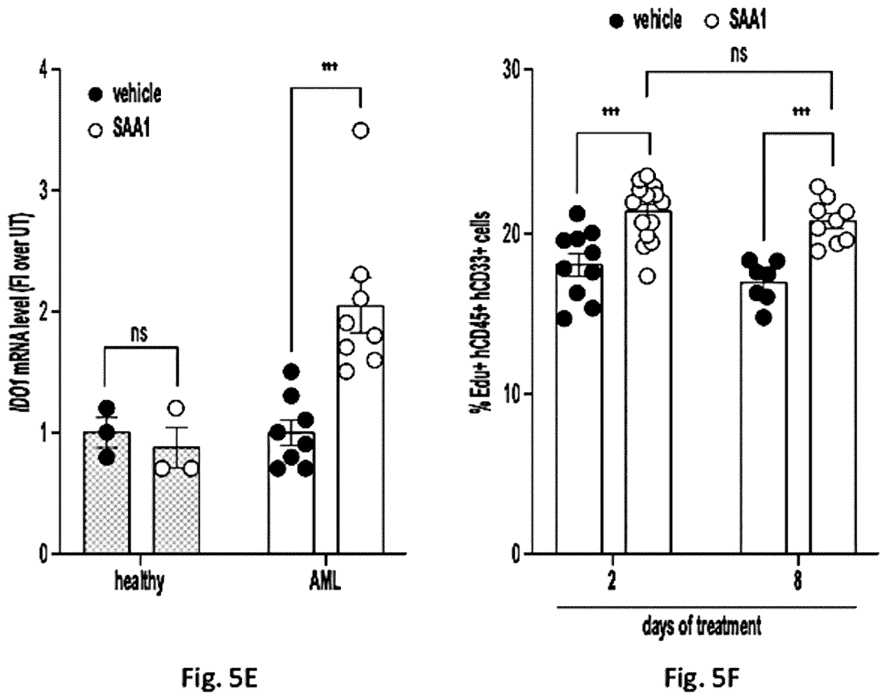

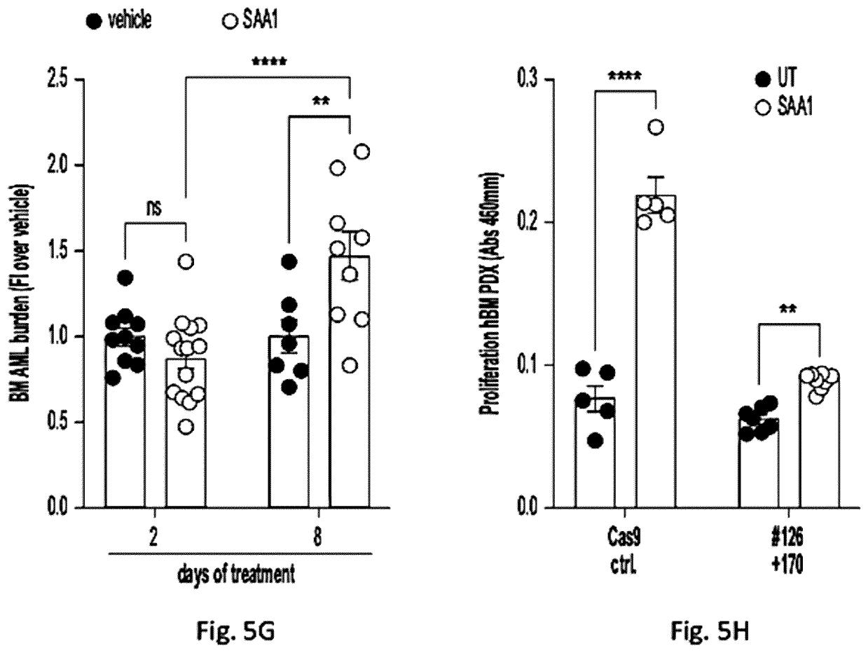

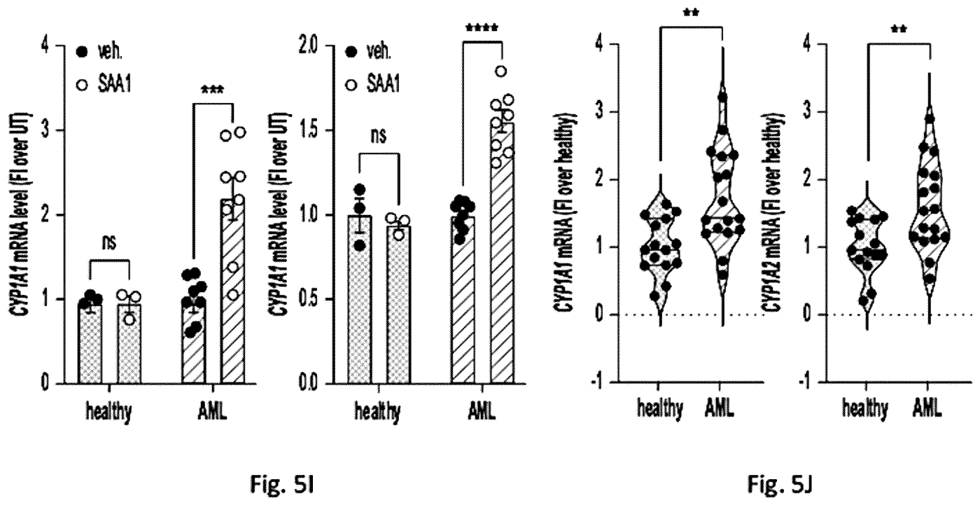

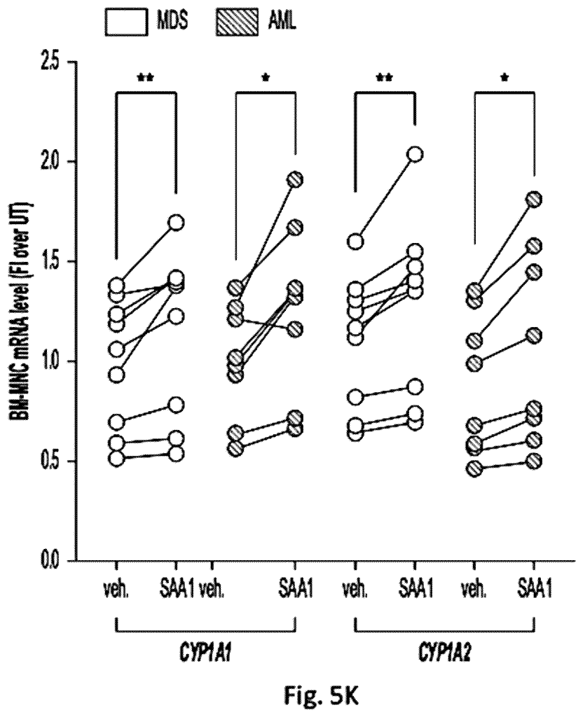

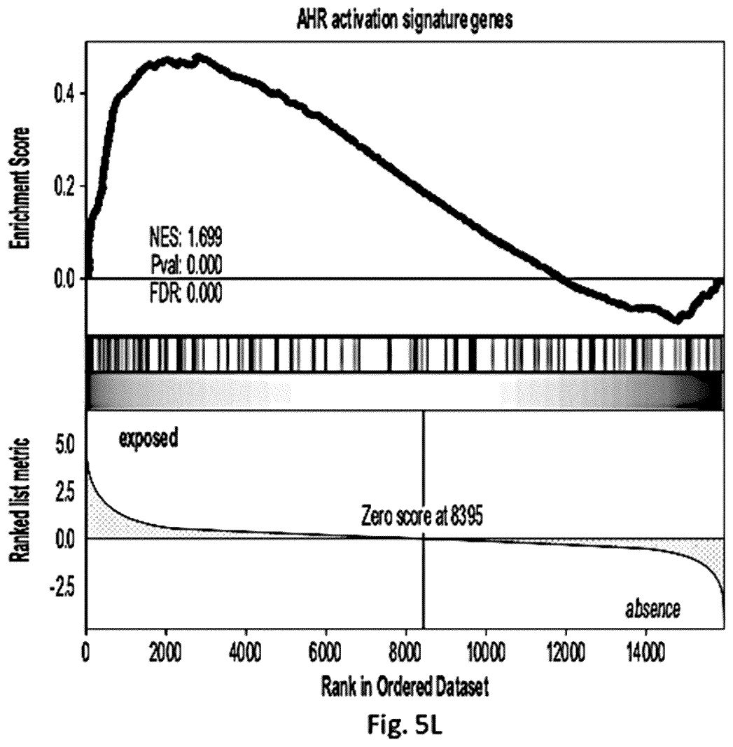

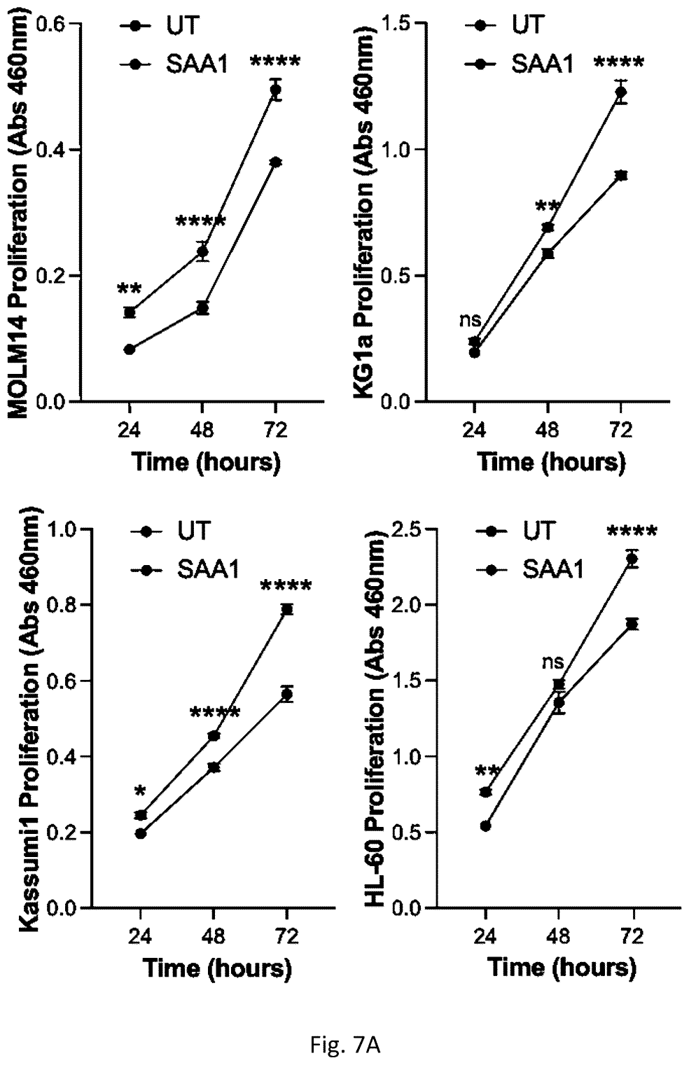

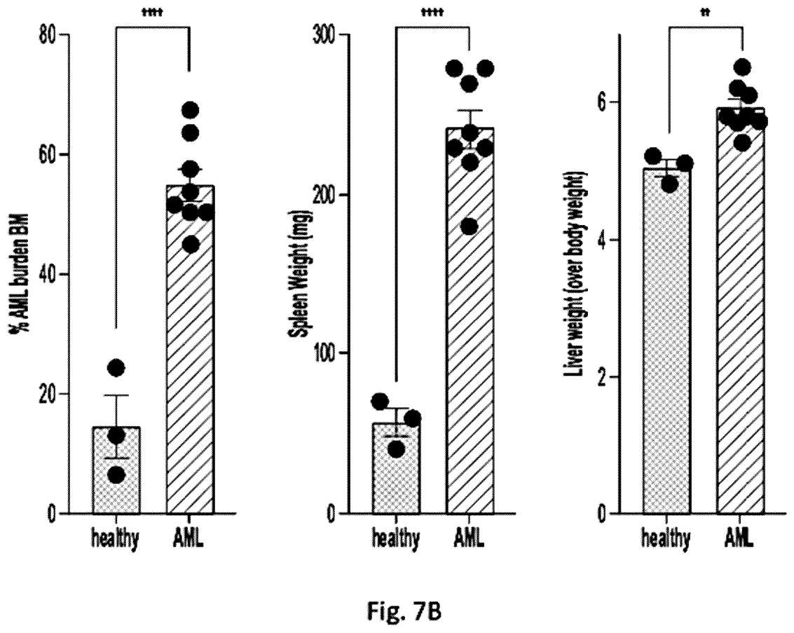

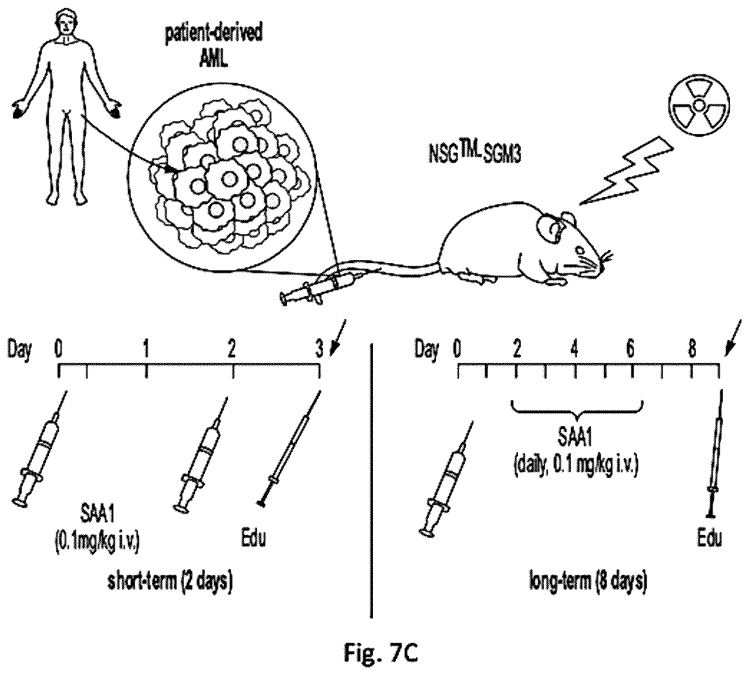

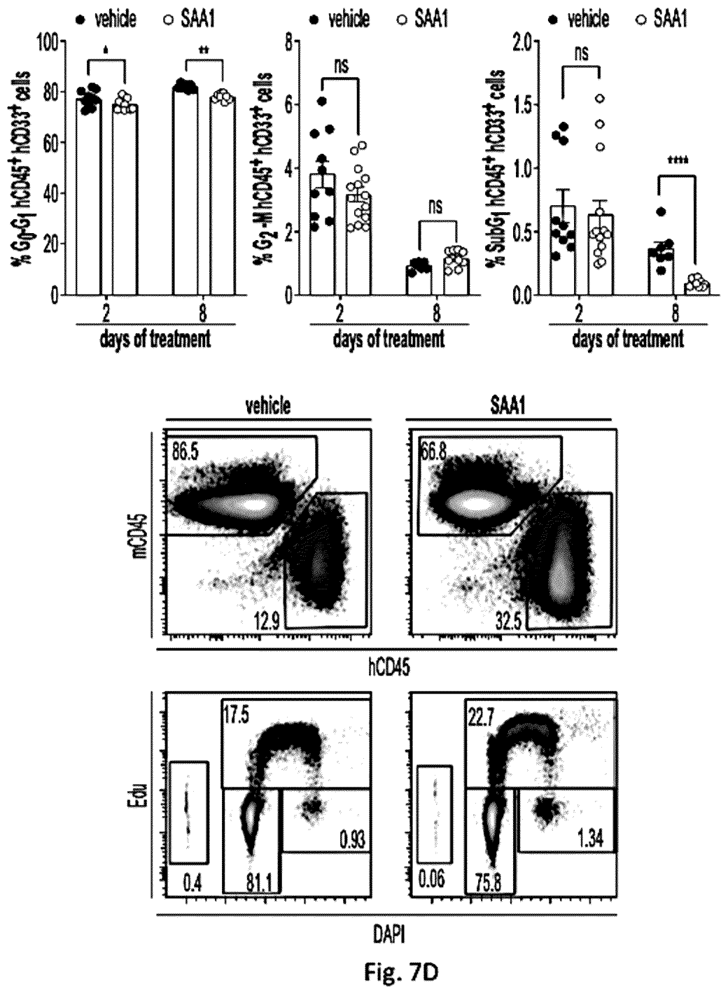

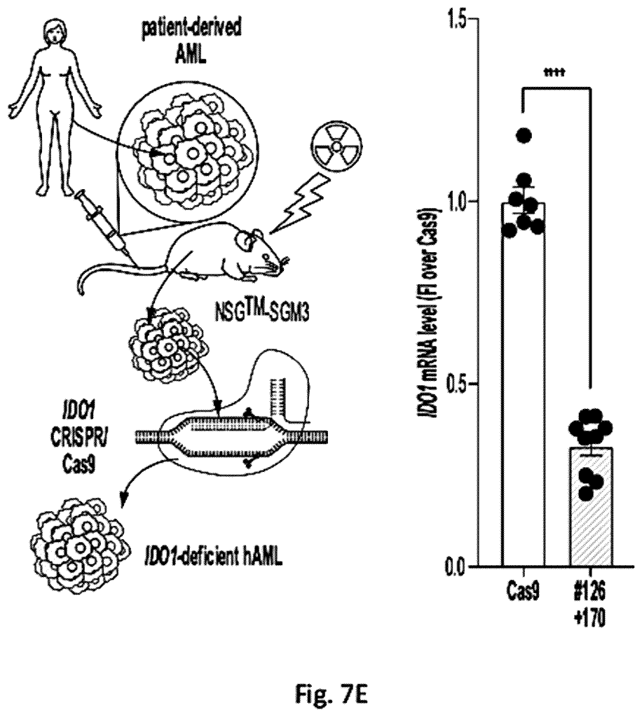

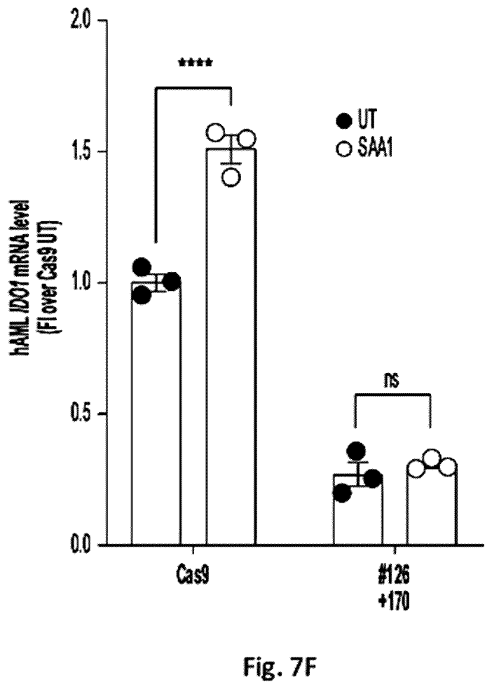

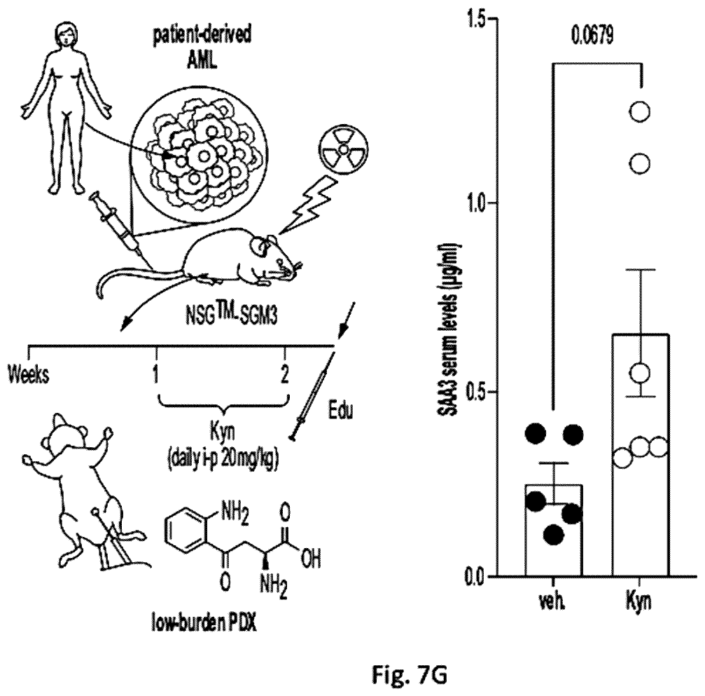

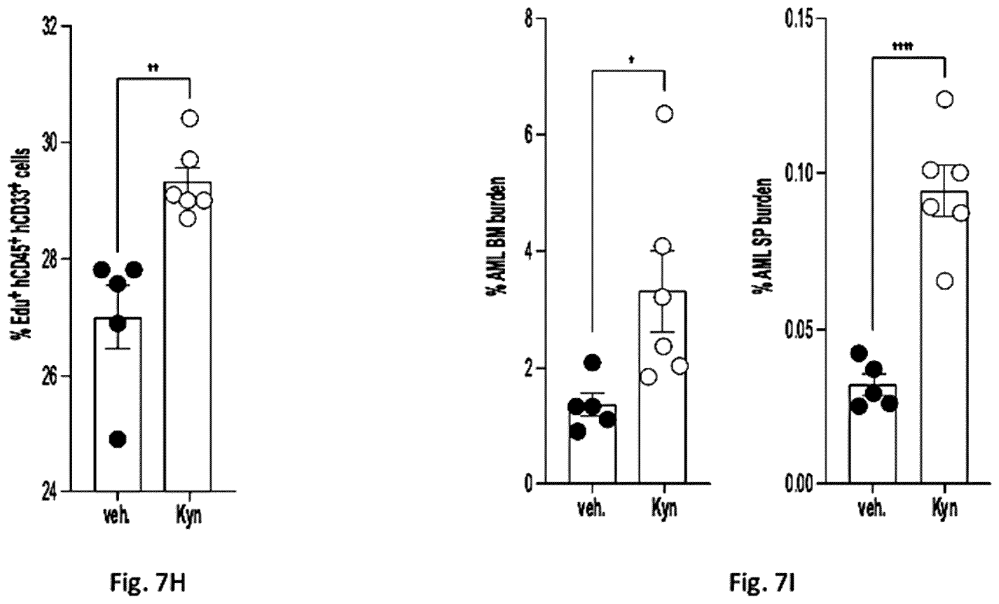

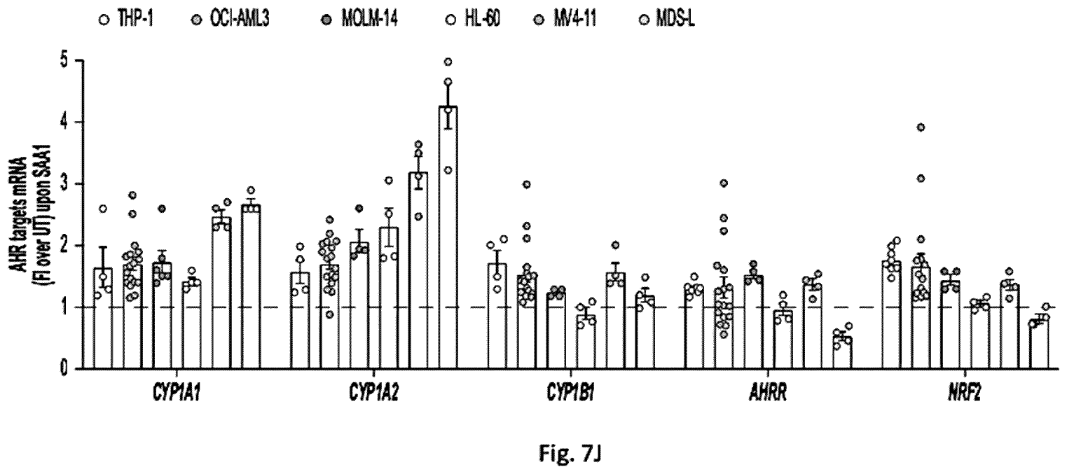

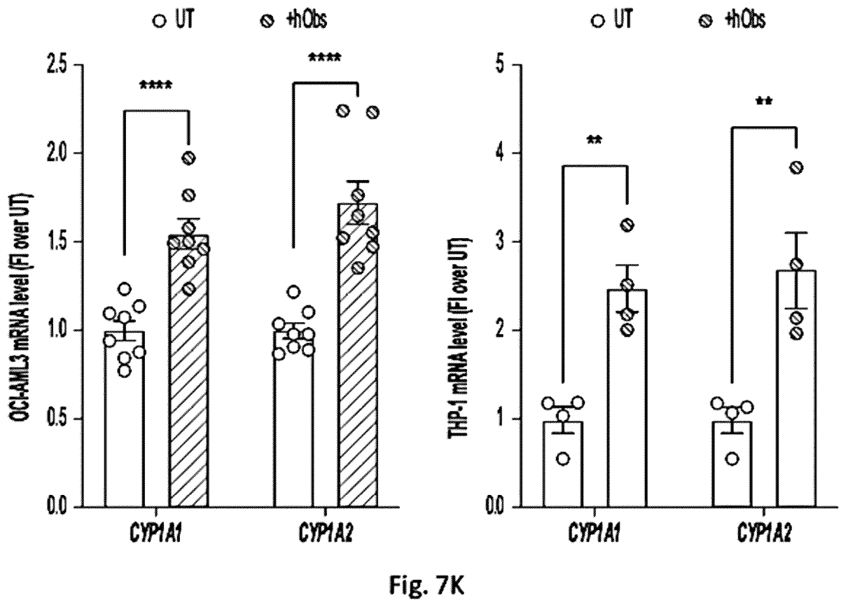

To this point, a compilation of data obtained from murine and human samples, and models of AML or MDS, demonstrate that leukemic cells stimulate a pro-inflammatory remodeling of the osteoblastic-niche. This mechanism may be a means for leukemia to operate a positive feedback loop that self-reinforces its progression, specifically through SAA1-mediated, HTR1B-dependent, upregulation of IDO1. To examine this hypothesis, we first tested the effect of SAA in leukemia cell proliferation. AML cell lines exposed to SAA1 (human) or SAA3 (mouse) showed an increased proliferation as compared to vehicle treated ones (FIG. 5A and FIG. 7A). Similarly, SAA1 promoted proliferation of lineage-depleted AML and MDS BM MNCs isolated from patient's aspirates (FIG. 5B). In parallel, IDO1 mRNA levels were increased in all the patient-derived BM MNCs upon SAA1 exposure (FIG. 5C).

To better understand the SAA-induced AML pro-proliferative activity in vivo, we took advantage of a patient-derived xenograft (PDX) model. Sublethally-irradiated NSG™-SGM3 (NSGS) mice were injected with either healthy human CD34+ cells (PDX healthy) or patient-derived AML cells (PDX AML), achieving a human engraftment range between 6-23% for the former, and 43-65% for the latter, 4 weeks after injection (FIG. 7B). Following BM isolation and CD45+ mouse cell depletion, total BM human cells were cultured and exposed to SAA1 for 24 h. While human cells from CD34+ healthy-injected mice were unresponsive to SAA1, patient-derived cells from AML-injected mice displayed a high proliferative activity as compared to their vehicle treated counterparts (FIG. 5D). Notably, IDO1 expression was only upregulated in response to SAA1 in the PDX-AML isolated human cells (FIG. 5E), mimicking our observations with patient-derived ex vivo cultures (FIG. 5C), and indicating that SAA1 induces at the same time IDO1 expression and proliferation of leukemia cells but not of healthy CD34+ cells.

To determine whether the SAA pro-proliferative activity observed in vitro and ex vivo was also reproduced in vivo, we treated PDX mice with recombinant human SAA1. SAA1 was administered i.v. at an equimolar dose to the one used for the in vitro and ex vivo assays for 2 or 8 days (FIG. 7C). Three hours before harvest, mice were injected with 5-ethynyl-2′-deoxyuridine (Edu) to analyze in vivo leukemic blasts cell cycle. SAA1 treatment yielded a maintained and prominent increase in the proliferative rate of leukemic blasts (hCD45+CD33+) after the 2- and the 8-day treatments as shown by the increase in Edu+ cells (S-phase; FIG. 5F) and the decrease in the G0-G1 cells while the G2-M phase was unvarying (FIG. 7D). In addition, 8-day treatment with SAA1 promoted survival of leukemic blasts (reduced the % of Sub-G1 apoptotic cells, FIG. 7D). Cumulatively, the increased proliferating rate and the decrease in apoptosis of leukemic blasts led to a 1.5-fold increase in AML burden in the BM at the completion of the 8-day treatment period (FIG. 5G).

To unequivocally assess whether the proliferation increase observed upon SAA exposure was a direct consequence of the concomitant upregulation of IDO1 expression, we performed CRISPR/Cas9 targeting of IDO1 in primary human AML cells isolated from the PDX model, achieving ˜70% deletion efficiency (FIG. 7E). Upon SAA1 exposure, IDO1-edited primary human AML cells failed to upregulate IDO1 expression as compared to control (Cas9 only) ones (FIG. 7F). More importantly, IDO1-targeted cells showed a 2-fold decrease in their proliferation rate in response to SAA1 as compared to control ones (FIG. 5H). Conversely, injection of Kyn in low AML burden PDX mice (to distinguish the stimulatory effect of exogenous Kyn from the one of AML cells) increases serum SAA3 (FIG. 7G) as well as the proliferative capacity of the leukemic blasts (FIG. 7H). As a consequence, AML burden increased in BM and SP of the Kyn-treated group (FIG. 7I).

These results suggest that SAA specifically promotes proliferation and cell cycle progression of leukemia cells. Moreover, SAA-induced proliferation occurs through upregulation of IDO1 expression.

### Example 28: SAA Engages the AHR Pathway to Increase IDO1 Expression

Since upregulation of IDO1 expression will trigger Kyn synthesis, we examined whether the Kyn-induced SAA1 secretion stimulates AML proliferation by activating Kyn signaling in AML cells. Kyn is an endogenous agonist of the aryl hydrocarbon receptor (AHR) (Opitz C A, et al. An endogenous tumour-promoting ligand of the human aryl hydrocarbon receptor. Nature. Nature Publishing Group; 2011; 478:197-203.), a ligand-activated transcription factor able to induce cell proliferation-reviewed in (Mulero-Navarro S, et al. New Trends in Aryl Hydrocarbon Receptor Biology. Front Cell Dev Biol. 2016; 4:45.)-. Therefore, we examined whether SAA1 induces AHR-dependent transcription of classical target genes. Indeed, exposure of human AML and MDS cell lines to SAA1 upregulated most of the main AHR targets genes (FIG. 7J). Similar to what we observed with IDO1 expression (FIG. 5E), CYP1A1 and CYP1A2 gene expression was only upregulated in response to SAA1 in the human BM cells isolated from the PDX-AML but not in the ones isolated from CD34+ healthy-injected mice (FIG. 5I), confirming the specificity of this mechanism for leukemic cells. We further confirmed these results in BM-MNCs of patients, showing that both AHR target genes were specifically upregulated in samples from AML patients but not from healthy subjects (FIG. 5J). Finally, we corroborated this AHR target gene activation pattern in the lineage-depleted AML and MDS BM MNCs isolated from patient's aspirates (FIG. 5K). Interestingly, an AHR activation signature was also upregulated in AML cells exposed to osteoblasts (FIG. 5L), and further confirmed in co-cultures of leukemic cells with human osteoblasts, which showed upregulation of the CYP1A1 and CYP1A2 genes upon osteoblasts exposure (FIG. 7K). All together, these data suggest that SAA production by osteoblasts upregulates IDO1 expression in AML cells through activation of the AHR pathway.

FIG. 5. SAA1 selectively promotes leukemic cell proliferation by upregulating IDO1 expression through activation of the AHR pathway. (A) Proliferation of human THP-1 and OCI-AML3 (n=22) and mouse WEHI-3B (n=8) AML cell lines exposed to SAA1 or SAA3 respectively (1 μg/ml, 24-72 h). Proliferation (B) and IDO1 mRNA levels (C) of human bone marrow mononuclear cells (BM-MNCs) isolated from MDS or AML (lineage-depleted) BM aspirates (n=8) and exposed to SAA1 (5 μg/ml, 24 h), paired t-test (D) Schematic of patient-derived xenograft (PDX) model used (left). Right: proliferation of total human BM cells isolated from the PDX mice injected with either healthy CD34⁺ (n=3) or patient-derived AML cells (n=8) exposed to vehicle (PBS) or SAA1 (1 μg/ml, 24 h). (E) IDO1 mRNA level from cells in (D); two-way ANOVA. In vivo proliferation of leukemic blasts (hCD45⁺CD33⁺) (F) and BM AML burden (G) in mice treated for 2 or 8 days with either vehicle (n=10 and n=7 respectively) or SAA1 (n=14 and n=9 respectively); 2-way ANOVA. (H) Proliferation of total human AML BM cells isolated from PDX mice and nucleofected with Cas9 (n=5) or Cas9 and the combination of sgRNA #126 (SEQ ID 82) and sgRNA #170 (SEQ ID 103) (n=8) exposed to vehicle or SAA1 (1 μg/ml, 24 h); two-way ANOVA. (I) mRNA level of CYP1A1 and CYP1A2 from cells in (D); two-way ANOVA. (J) Violin plots for mRNA levels of CYP1A1 and CYP1A2 in BM-MNCs from healthy (n=15) and AML (n=17) patients. (K) CYP1A1 and CYP1A2 mRNA levels from cells in (B). (L) GSEA analysis of AHR activation signature genes in THP-1 cells co-cultured with human osteoblasts for 24 h. All data expressed as mean±SEM. Statistical analysis was done with unpaired t-test unless otherwise stated. See also FIG. 7.

FIG. 7. SAA1 selectively promotes AML cell proliferation. Related to FIG. 5. (A) Proliferation of human AML cell lines (MOLM-14, KG-1a, Kasumi-1 and HL-60) exposed to SAA1 (1 μg/ml) for 24, 48 or 72 h, (n=8 for all cell lines); two-way ANOVA. (B) AML burden, spleen weight and liver weight (over body weight) in the PDX mice 4 weeks after transplant with either CD34⁺ healthy (n=3 mice) or patient-derived AML (n=8 mice) cells. (C) Diagram showing the short term (2-days) vs long-term (8-days) SAA1 in vivo treatments. (D) In vivo cell cycle analysis showing % of cells in G₀-G₁, G₂-M and Sub-G within the leukemic blasts (hCD45⁺CD33⁺) comparing 2-day vs 8-day treatments, in vehicle-(n=10 and n=7 respectively) or SAA1-treated (0.1 mg/kg; n=14 and n=9 respectively); 2-way ANOVA. On the right, representative flow-plots for BM AML burden (top) and proliferation analysis (bottom) in the 8-day treatment group. (E) Schematic of CRIPSR/Cas9 targeting of PDX-isolated AML human cells (left) and IDO1 mRNA level in human AML cells nucleofected with Cas9 (n=7) or Cas9 and the combination of sgRNA #126 (SEQ ID 82) and sgRNA #170 (SEQ ID 103) (n=9). (F) IDO1 mRNA level of cells in (E) cultured for 24 h with either vehicle or SAA1 (1 μg/ml), (n=3); two-way ANOVA. (G) Schematic of Kyn treatment in low-burden PDX (left) and SAA3 serum levels in NSGS mice injected with vehicle (n=5) or Kyn (20 mg/kg; n=6) for 1 week. (H) Percentage of blasts (hCD45⁺hCD33⁺) Edu⁺ cells of mice in (G). (I) AML burden in BM and SP of mice in (G). (J) mRNA level (expressed as FI over basal level in untreated cells: red line) of main AHR target genes in the indicated human AML and MDS cell lines exposed to SAA1 (1 μg/ml) o/n (n=4-8 for all cell lines except OCI-AML3 n=17). (K) mRNA level (FI over UT) of AHR targets in OCI-AML3 and THP-1 cells exposed to primary human osteoblasts for 24 h; 2-way ANOVA. All data expressed as mean±SEM. Statistical analysis was done with unpaired t-test unless otherwise stated.

### Example 29: Pharmacological Targeting of the Kynurenine-HTR1B-SAA-IDO1 Axis in Xenografts Impairs AML Proliferation

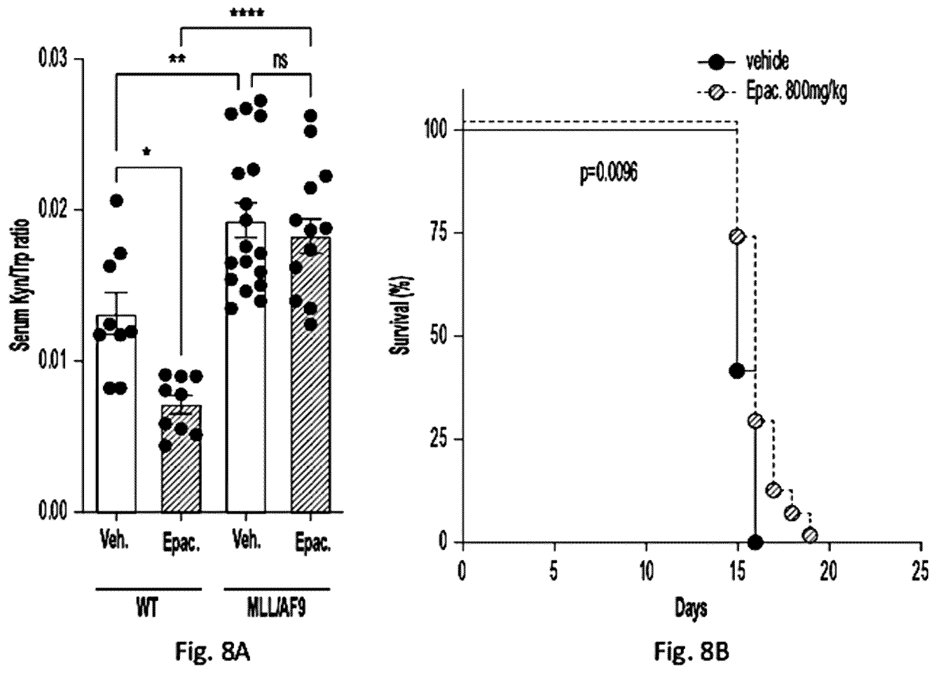

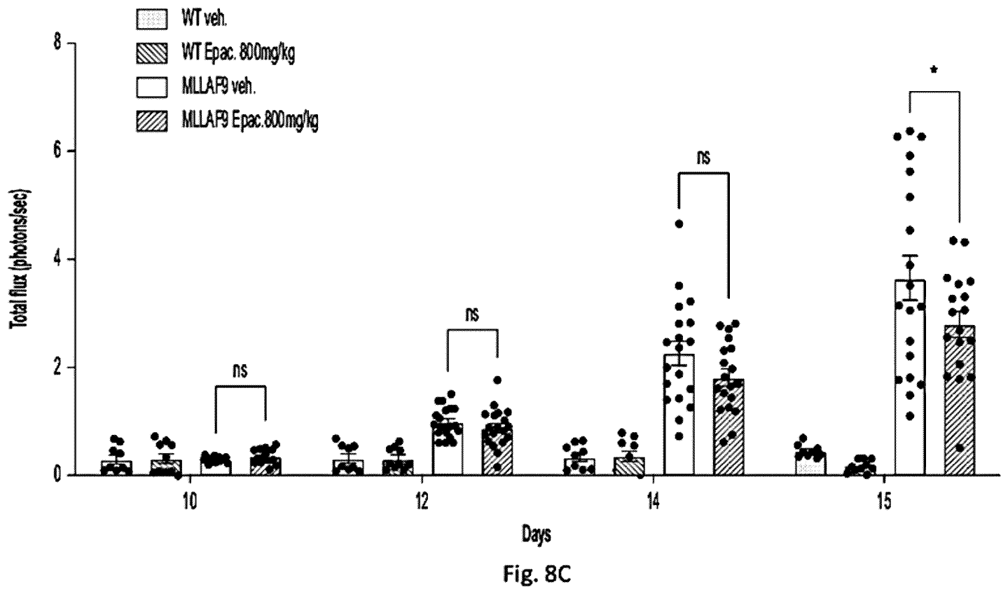

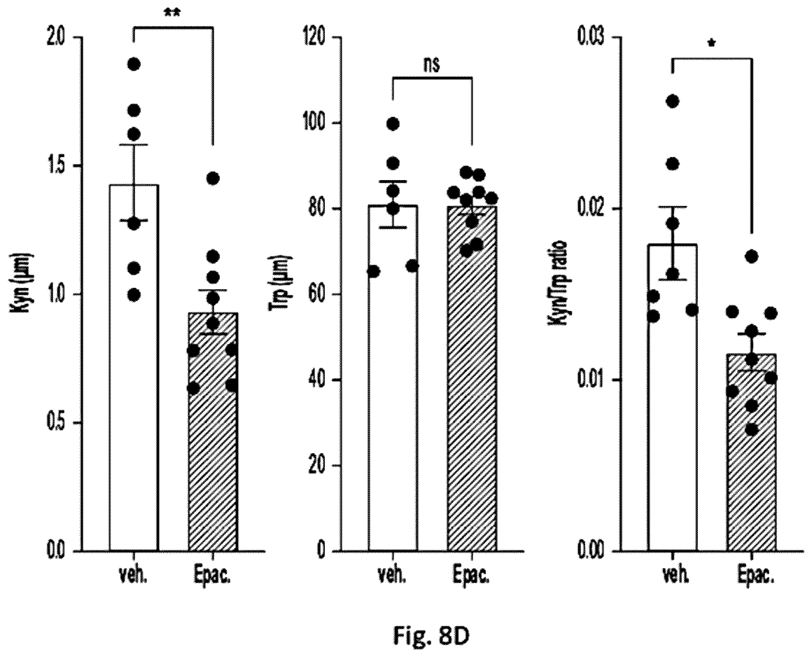

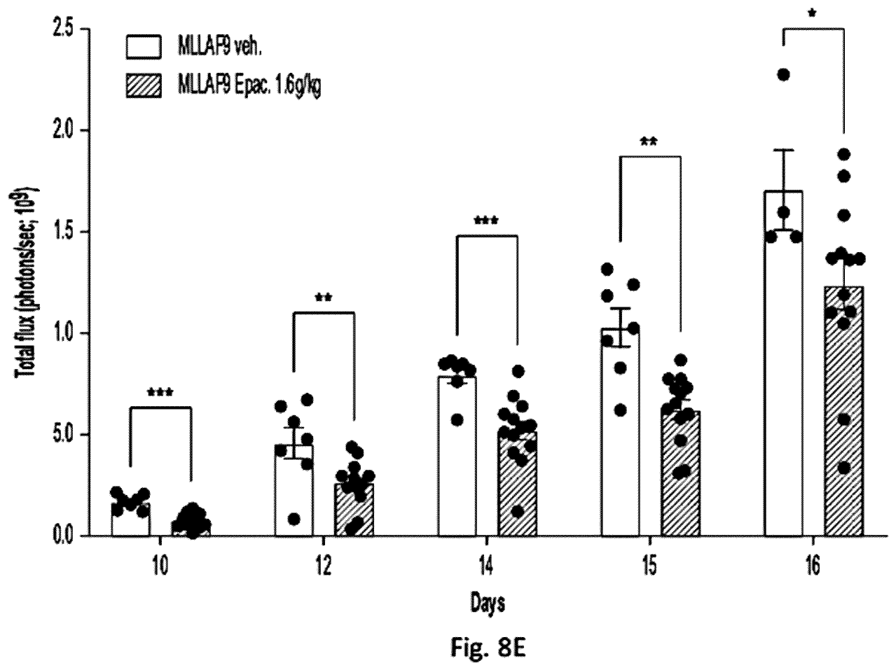

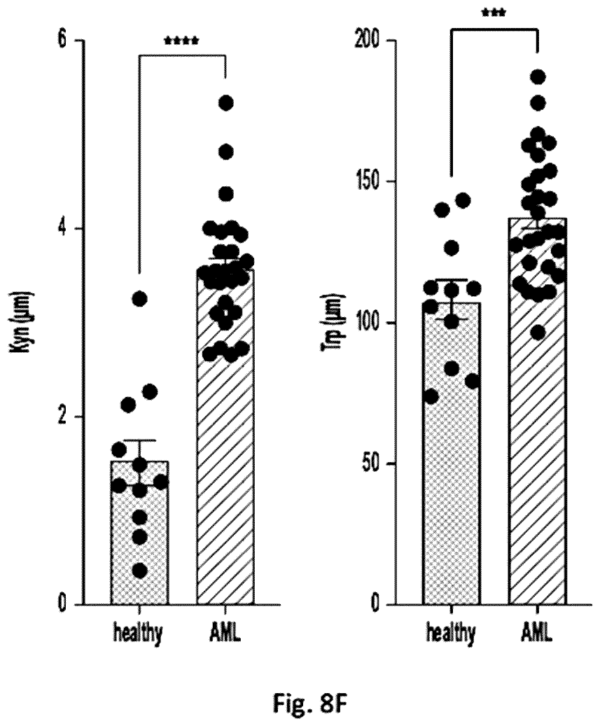

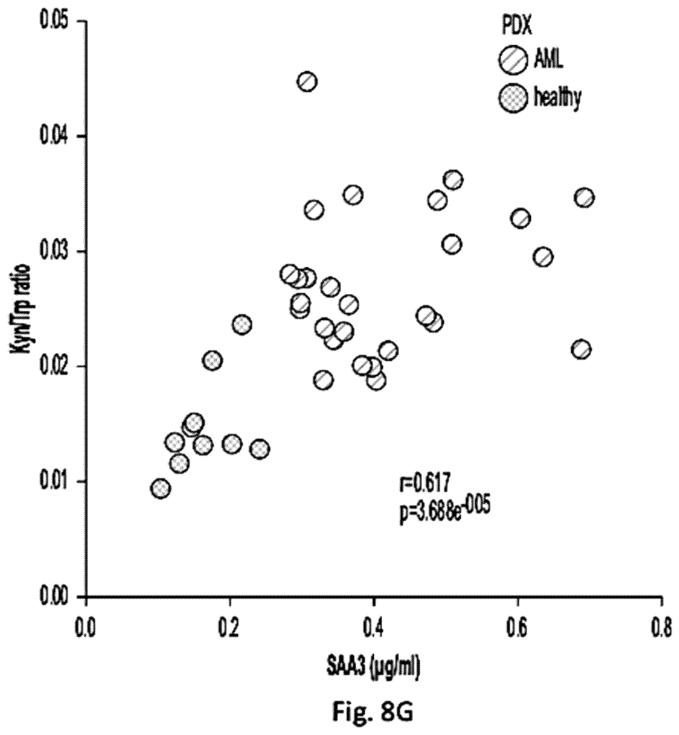

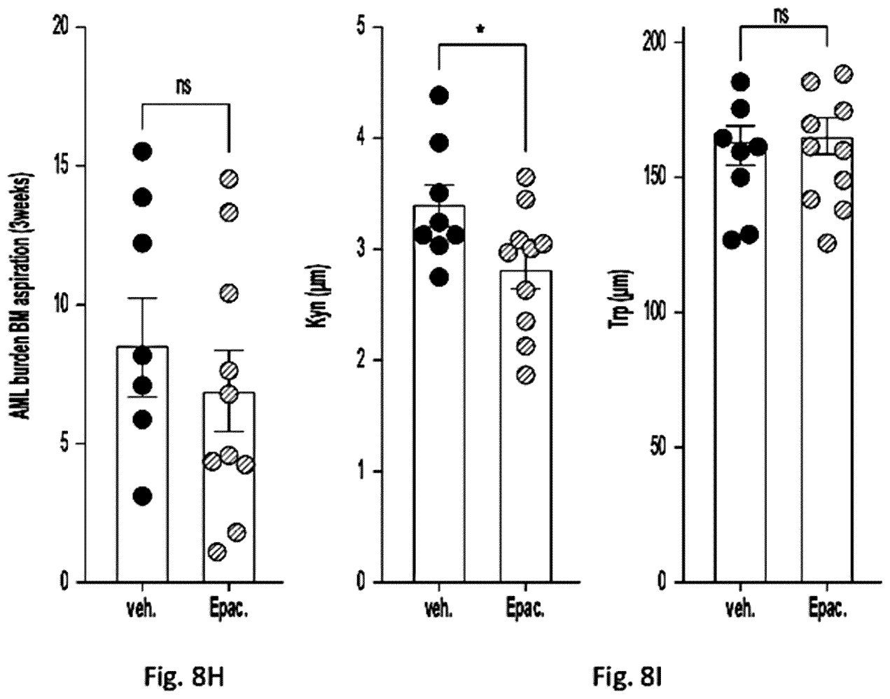

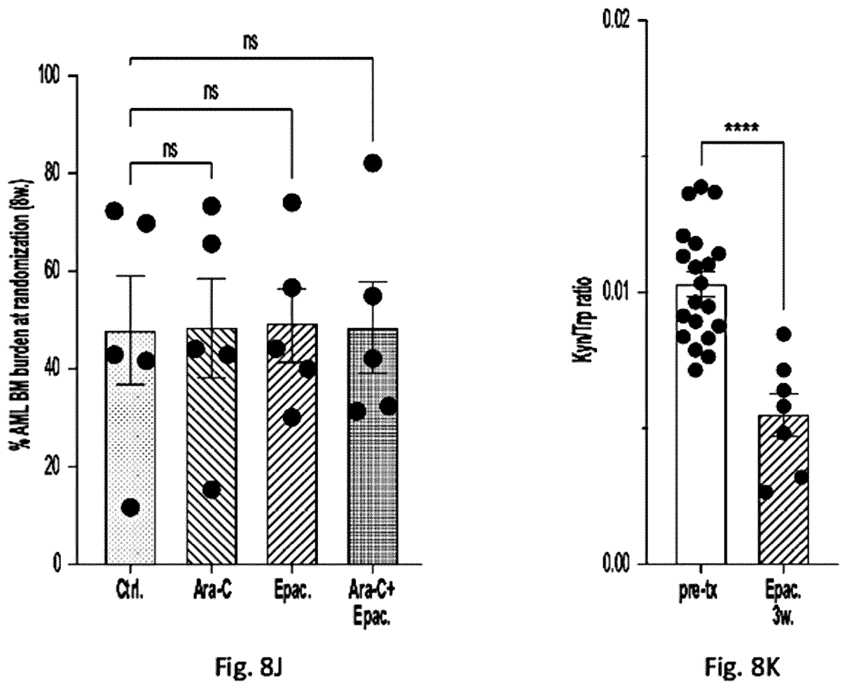

The demonstration that IDO1 ablation has potent anti-leukemic effects prompted us to explore the therapeutic potential of inhibiting IDO1 activity for leukemia growth. Therefore, we analyzed the effect of epacadostat, a potent, selective and competitive inhibitor of IDO1 enzymatic activity (Liu X, et al. Selective inhibition of IDO1 effectively regulates mediators of antitumor immunity. Blood. 2010; 115:3520-30,46 and Koblish H K, et al. Hydroxyamidine inhibitors of indoleamine-2,3-dioxygenase potently suppress systemic tryptophan catabolism and the growth of IDO-expressing tumors. Molecular Cancer Therapeutics. 2010; 9:489-98.), in leukemia progression. WT mice receiving epacadostat in an ad libitum diet (0.8 g/kg) showed a 54% reduction in their basal (no leukemia) circulating Kyn/Trp levels (FIG. 8A) and, consistent with previous reports (Yue E W, et al. INCB24360 (Epacadostat), a Highly Potent and Selective Indoleamine-2,3-dioxygenase 1 (IDO1) Inhibitor for Immuno-oncology. ACS Med Chem Lett. 2017; 8:486-91.), did not show any obvious systemic toxicity. In the MLL/AF9 leukemic mice, we observed a slight-yet significant-increase in survival when mice were treated with epacadostat (FIG. 8B). However, despite a tendency towards a slower leukemia progression (FIG. 8C), the in vivo pharmacology of epacadostat at the selected dose, was such that it did not reduce the 1.5-fold-increase in systemic Kyn/Trp levels observed in the MLL/AF9 leukemic mice (FIG. 8A). Doubling the epacadostat dose (1.6 g/kg) resulted in a 35% reduction in serum Kyn levels (FIG. 8D), a significant delay in AML burden (FIG. 8E) and a significantly prolonged survival as compared to the lower dose (FIG. 6A). Hence, reduction in Kyn levels proportionally affects leukemia burden and overall survival.

Subsequently, we investigated the effect of pharmacological inhibition of the Kyn pathway in a clinically relevant PDX model of human AML. First, we verified that regulation of the Kyn-HTR1B-SAA axis is reproduced in response to AML in xenografts. Consistent with our observations in murine models and patient samples, immunodeficient (NSGS) mice transplanted with patient-derived human AML cells showed higher SAA3 (FIG. 6B) and Kyn/Trp ratio (FIG. 6C and FIG. 8F) peripheral levels as compared to PDX mice transplanted with CD34+ healthy cells. Moreover, mirroring our observations in patient samples (FIG. 4I), we observed a positive correlation between both biomarkers and disease state (FIG. 8G). These results not only demonstrate the conserved response and activation of this axis in mammals, but they also reinforce the notion for assessment of both Kyn and SAA1 as biomarkers in the diagnosis of AML progression.

Patient-derived de novo AML cells were injected into sublethally irradiated NSGS mice, (FIG. 6D) and 3 weeks after transplantation BM aspiration was performed to randomize the groups (FIG. 8H). To control the daily intake of epacadostat, we opted for a 12-day regime of daily gavage (300 mg/kg). While achieving only a ˜20% reduction in Kyn/Trp levels in blood (FIG. 6E and FIG. 8I)—likely owing to the short duration of the treatment-, epacadostat-treated animals showed a concomitant ˜20% reduction in AML BM burden compared to the vehicle treated group (FIG. 6F). Moreover, in vivo assessment of the leukemic blast (hCD45+CD33+) cell cycle showed that epacadostat-treated leukemic blasts were less proliferative than the vehicle-treated ones (FIG. 6G). Interestingly, while the G0-G1 and G2-M populations remained unchanged, epacadostat treatment increased leukemic blasts apoptosis (FIG. 6H), contrary to the anti-apoptotic effects observed upon SAA1 treatment (FIG. 11D).

The therapeutic potential of targeting the kynurenine-HTR1B-SAA-IDO1 axis in an established PDX leukemia model was studied by inhibiting Kyn synthesis as an adjuvant treatment for chemotherapy (FIG. 6I). 8 weeks after transplant, at the time of randomization, BM aspiration showed ˜50% AML burden (FIG. 8J). Leukemic mice were then treated for 3 weeks with control chow, chemotherapy alone (Ara-C for 5 days; (48)), epacadostat diet (ad libitum, 1.6 g/kg) or combination therapy (Ara-C+Epacadostat). As previously described in this model (48), leukemic burden is decreased by day 8 after initiating therapy with single agent Ara-C, but relapse occurs consistently between 22-29 days after starting therapy (FIG. 6I). As expected with assessment at day 22, although not significant (probably due to the low number of mice), Ara-C alone treated mice had a modest decrease in overall leukemic burden likely indicating that relapse is underway (FIG. 6J). The effect of Ara-C was more sustained in the spleen than in BM in agreement with previous results in this model (48,49). Consistent with results above (FIG. 6F), epacadostat as a standalone intervention also decreased leukemic burden in the BM of NSG mice (FIG. 6J and FIG. 8K), and as Ara-C, it had a more pronounced effect in the spleen (FIG. 6K). Importantly, we assessed the effect of epacadostat/Ara-C combination at day 22 after initiating therapy. The combination treatment significantly decreased leukemic burden in the BM and spleen, although this effect was synergistic only in the BM (FIGS. 6J and 6K). Thus, epacadostat inhibition of IDO1 enhances the response to Ara-C in this pre-clinical model.

Collectively, our results reveal that leukemia cells subvert serotonin signaling in osteoblasts, inducing a self-perpetuating pro-inflammatory niche by exploiting the kynurenine-HTR1B-SAA-IDO1 axis (FIG. 6L). These results provide strong evidence for a central role of the kynurenine-HTR1B-SAA-IDO1 axis in human AML progression. Moreover, they provide proof-of-principle that targeting this axis can be therapeutically beneficial in ways that complement standard induction therapies and described immunosuppressive effects of Kyn.

FIG. 6A-6L shows the results of pharmacological targeting of the kynurenine-HTR1B-SAA-IDO1 axis in patient-derived xenografts. (A) Survival curve comparing vehicle (n=26), and epacadostat-treated mice (n=18 for 0.8 g/kg and n=13 for 1.6 g/kg). Kaplan-Meier curve with p-value of log rank (Mantel-Cox) test. SAA3 (B) and Kyn/Trp ratio (C) serum levels in NSGS mice transplanted with CD34⁺ healthy cells (n=11) or with patient-derived AML cells (n=27). (D) Schematic describing pharmacological targeting of IDO1 (epacadostat) in patient-derived AML xenograft (PDX) in NSGS mice. (E) Kyn/Trp ratio in serum of PDX mice 5 weeks after AML transplant and 2 weeks post-epacadostat treatment (n=8 vehicle; n=10 epacadostat). (F) Representative flow cytometry plots depicting % of human or mouse CD45⁺ cells in the BM of PDX mice (left) and AML burden in the BM of PDX mice at harvest (right) (n=8 vehicle; n=10 epacadostat). (G) Representative flow cytometry plots (left) and cell cycle analysis of leukemic blasts (CD45⁺CD33⁺) of PDX mice treated with either vehicle (n=8) or epacadostat (n=8). (H) Cell cycle analysis of mice in (G). (I) Schematic diagram showing the in vivo PDX mouse model treated with the combination therapy (Ara-C 60 mg/kg 1-5 days+Epacadostat 1.6 g/kg ad libitum 3 weeks). AML burden in BM (J) and spleen (K) 11 weeks after transplant, 3 weeks after combination therapy; control chow (ctrl. n=4), Ara-C (n=3), Epacadostat (Epac. n=4) and combination therapy (Ara-C+Epac. n=3); one-way ANOVA; unpaired t-test p values are shown for BM ctrl vs Ara-C and Epac groups. (L) Schematic model of the kynurenine-HTR1B-SAA-IDO1 axis depicting the AML-mediated osteoblastic self-reinforcing niche remodeling. All data expressed as mean±SEM. Statistical analysis done with unpaired t-test unless otherwise stated. See also FIG. 8.

FIG. 8. Epacadostat hampers AML progression. Related to FIG. 6. (A) Kyn/Trp ratio levels in WT mice injected or not with MLL/AF9 cells and treated with either vehicle or epacadostat (no leukemia: vehicle n=9, Epac. n=9; MLL/AF9-injected mice: vehicle n=18, Epac. n=14). One-way ANOVA. (B) Survival curve comparing leukemic mice treated with either vehicle (n=19) or epacadostat (n=19); Kaplan-Meier curve with p-value of log rank (Mantel-Cox) test. (C) In vivo leukemia burden quantification of mice shown in (A), treated with either vehicle or 0.8 g/kg epacadostat. (D) Kyn and Trp absolute levels and Kyn/Trp ratio in serum of WT mice injected with MLL/AF9 cells and treated with either vehicle (n=6) or 1.6 g/kg ad libitum epacadostat diet (n=9). (E) In vivo leukemia burden quantification of mice in (D). (F) Kyn and Trp absolute levels in serum of NSGS mice transplanted with either healthy CD34⁺ cells (n=11) or patient-derived AML cells (n=27). (G) Multiple variable data plot of SAA3, Kyn/Trp ratio serum levels and transplanted disease in NSGS mice transplanted with CD34⁺ healthy cells (n=11) or with patient-derived AML cells (n=27), Pearson correlation values are shown for Kyn/Trp ratio and SAA3 serum levels. (H) AML burden in BM aspirates from PDX in NSGS mice at randomization (3 weeks; n=8 vehicle, n=10 epacadostat). (I) Kyn and Trp levels in serum of PDX mice at harvest 5 weeks after transplant and 12 days post-epacadostat treatment (n=8 vehicle, n=10 epacadostat). (J) AML burden in BM aspiration 8 weeks after transplant of PDX NSG mice at randomization (n=5 for all groups). (K) Kyn/Trp ratio serum levels in all mice before treatment (n=20) and after 3 weeks of epacadostat diet (n=7). All data expressed as mean±SEM. Statistical analysis was done with unpaired t-test unless otherwise stated.

### Example 30: Inhibition of SAA1 Proliferation Assays

Monoclonal antibodies will be prepared against SAA1 using standard hybridoma techniques. Supernatants of the potential clones will be tested for their blocking ability in luciferase-reporter assays. The stable murine macrophage RAW 264.7 NFκB-Luc cells will be exposed to SAA1 in a dose-response and time-dependent manner to optimize the initial assay. After determining the optimal dosages of positive control (lipopolysaccharide, LPS), anti-SAA1 and duration of cells will be treated with the received antibody subclones, to assess their ability to block LPS and/or SAA1 NFκB activation.

Cell proliferation will be performed by using Cell Counting Kit 8 (WST-8, Abcam) as per manufacturer's instructions. Briefly, 0.03×10⁶ cells will be seeded on tissue-culture clear bottom microplates (Corning) in their corresponding media (100 μl). When indicated, cells will be treated with the indicated compounds and for the indicated time points. 10 μl/well of WST-8 solution will be added and incubated for 2 h at 37° C. before measuring absorbance at 460 nm. For each experiment, the absorbance of the blank wells (growth media and vehicle/treatment) will be subtracted from the values for those wells with cell In vitro: the indicated cell lines will be incubated in reduced-serum media and exposed to SAA1 (1 μg/ml) or SAA1+anti-SAA1 monoclonal antibodies for the 24-72 h as indicated.

Ex vivo xenografts (healthy CD34⁺ versus patient-derived AML): total BM from NSGS mice will be depleted of mouse cells with mouse CD45 magnetic beads (Miltenyi Biotec Cat #130-052-301, RRID: AB_2877061) and will represent negatively selected human cells to be used.

Ex vivo primary AML and MDS patient's samples: MNCs from fresh BM patients' aspirates will be isolated as previously described and depleted from mature hematopoietic cells (lineage Cell Depletion Kit, Miltenyi Biotec Cat #130-092-211). Isolated cells will be seeded on StemMACS HSC Expansion Media XF supplemented with StemMACS HSC Expansion Cocktail (Miltenyi Biotec, Cat #130-100-463 & 130-100-843) and then will be treated with either vehicle (PBS), SAA1 (5 μg/ml).

Monoclonal anti-SAA1 will inhibit SAA1 proliferation of the leukemic cells in a dose dependent manner. Specifically, blocking anti-SAA1 antibodies will show 1) anti-proliferative effect specifically to the targeted leukemic cells (i.e., not affect healthy ones), 2) broad applicability (not limited to the mutational landscape), and 3) prevention of relapse by disruption of the AML-niche crosstalk hijacked by leukemia to grow.

### Statistical Analysis

Sample size determination for in vivo experiments was estimated by considering a multifactorial variance analysis; a n=5 minimum number of mice assigned to each treatment group would reach a power of 0.85. The Type I error probability associated with our tests of the null hypothesis was 0.05. Samples and mice were assigned to the different experimental groups in a random fashion. Male and female mice were used. Investigators were unblinded. Blinding during animal experiments was not possible because mice underwent a specific leukemia injection diet supply and/or daily treatment. No data were excluded from the study. We confirm that all experiments were reproducible by repeating them a minimum of 2-times-generally 3-4-using different stocks of cell lines, patient or mouse samples and reagents. All single data points in all figures represent biological replicates, from separate mice, separate experiments (cell lines) or, in the case of primary cultures of human or murine osteoblasts, measurements were performed on independently grown cultures. In the case of human data, each data point corresponds to an independent patient sample. The binding experiments were reproduced by two independent groups at the Department of Psychiatry, Columbia University, (Dr. M. Quick) and at Division of Chemical Biology and Medicinal Chemistry, Eshelman School of Pharmacy, University of North Carolina at Chapel Hill (Dr. B. Roth). Replication of experiment details are found in each figure legend.

Statistics: All numerical results are reported as mean±SEM. Data fits of binding isotherms were performed using nonlinear regression analysis in GraphPad Prism (RRID:SCR_002798) and the best-fit values and errors represent the mean and SEM of the fit. All numerical values used for graphs and detailed statistical analysis can be found in the figure legends as well as summarized in Table 5. Data assumed normal distribution, and so statistical significance of the difference between experimental groups was analyzed mainly with one-way ANOVA, two-way ANOVA, and unpaired t-tests were used, depending on the number of groups and conditions, unless otherwise stated in the figure legend. Differences were considered statistically significant for p≤0.05 and denoted as: *p≤0.05; **p≤0.01; ***p≤0.001; ****p≤0.0001.

The values shown in Table Sare the mean±standard error of the mean (SEM). Half maximal inhibitory concentration (IC50); inhibitory constant (Ki); half maximal effective concentration (EC50); 5-hydroxytryptophan (5-HT); kynurenine (Kyn).

Data analysis software: All statistical analyses were performed with GraphPad Prism 9 (RRID:SCR_002798) software. In vivo quantification of leukemia progression was performed with Living Image v4.7.2 (Perkin Elmer, RRID:SCR_014247). Confocal images were analyzed using ImageJ (RRID:SCR_003070) software. Metabolomic data analysis was performed using Matplotlib for Python (RRID:SCR_008624). Flow cytometry data analysis was performed using FlowJo (RRID:SCR_008520) software. CRIPSR editing analysis was performed with the Synthego Performance Analysis, ICE Analysis. 2019. v2.0. Synthego. Biorender was used to create all the diagrams, cartoons and schematics shown along the manuscript, under the Columbia University academic license. RNAseq data analysis, was done using the following software: STAR 2.7 (RRID:SCR_004463), featurecounts 1.6.5 (RRID:SCR_012919), R 3.6.3, Python 3.7.3 (IPython, RRID:SCR_001658) and GSEApy 0.9.18.

Data Availability: The RNA sequencing data generated during this study are publicly available in Gene Expression Omnibus (GEO) at GSE154374 (RRID:SCR_005012). Original/source data for FIG. 9A is available at Protein Data Bank (#6E45, https://www.rcsb.org/structure/6E45). Derived data supporting the findings in FIG. 2I are shown in Table 1.

The foregoing description of the specific implementations will so fully reveal the general nature of the disclosure that others can, by applying knowledge within the skill of the relevant art(s), readily modify and/or adapt for various applications such specific implementations, without undue experimentation, without departing from the general concept of the present disclosure. Such adaptations and modifications are therefore intended to be within the meaning and range of equivalents of the disclosed implementations, based on the teaching and guidance presented herein. It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one skilled in the relevant art(s). It is to be understood that dimensions discussed or shown are drawings accordingly to one example and other dimensions can be used without departing from the disclosure.

The subject matter described above is provided by way of illustration only and should not be construed as limiting. Various modifications and changes can be made to the subject matter described herein without following the example embodiments and applications illustrated and described, and without departing from the true spirit and scope of the invention encompassed by the present disclosure, which is defined by the set of recitations in the following claims and by structures and functions or steps which are equivalent to these recitations.

TABLE 1

| Sample | | IPSSR Risk | | | |
|---|---|---|---|---|---|
| ID | WHO 2016 | Category | TPH1 | IDO1 | Ratio IDO1/TPH1 |
| MDS1 | MDS-EB-1 | High | 1.52 | 34.78 | 22.88157895 |
| MDS2 | MDS-MLD | Low | 0.39 | 17.96 | 46.05128205 |
| MDS3 | MDS-EB-2 | Very high | 0.42 | 32.96 | 78.47619048 |
| MDS4 | MDS-EB-1 | Intermediate | 0.60 | 23.06 | 38.43333333 |
| MDS5 | MDS-MLD | Low | 0.43 | 26.23 | 61 |
| MDS6 | MDS-EB-1 | High | 0.79 | 27.12 | 34.32911392 |
| MDS7 | MDS-MLD | Low | 0.99 | 20.53 | 20.73737374 |
| MDS8 | MDS-EB-2 | Intermediate | 0.99 | 20.55 | 20.75757576 |
| MDS9 | MDS-MLD | Intermediate | 0.55 | 33.20 | 60.36363636 |
| MDS10 | MDS-EB-1 | Intermediate | 0.50 | 20.36 | 40.72 |
| MDS11 | MDS-EB-1 | Intermediate | 0.35 | 22.90 | 65.42857143 |
| MDS12 | MDS-MLD | Low | 0.56 | 21.26 | 37.96428571 |
| MDS13 | N/A | N/A | 0.89 | 27.64 | 31.05617978 |
| MDS14 | MDS-EB-2 | Very high | 0.76 | 24.39 | 32.09210526 |
| MDS15 | MDS-EB-2 | Very high | 1.43 | 22.38 | 15.65034965 |
| MDS16 | MDS-MLD | High | 0.58 | 16.83 | 29.01724138 |
| MDS17 | MDS-EB-2 | Very high | 0.72 | 24.39 | 33.875 |
| MDS18 | MDS-MLD | N/A | 0.91 | 31.95 | 35.10989011 |
| MDS19 | MDS-EB-2 | N/A | 0.52 | 21.13 | 40.63461538 |
| MDS20 | MDS/MPN | N/A | 0.41 | 20.55 | 50.12195122 |
| MDS21 | MDS-MLD | Intermediate | 1.15 | 21.57 | 18.75652174 |
| MDS22 | MDS-EB-2 | High | 0.83 | 27.42 | 33.03614458 |
| MDS23 | MDS-EB-1 | N/A | 0.42 | 19.78 | 47.0952381 |
| MDS24 | MDS-MLD | Low | 0.90 | 22.81 | 25.34444444 |
| MDS25 | MDS-MLD | Intermediate | 1.52 | 40.39 | 26.57236842 |
| MDS26 | MDS-EB-2 | Intermediate | 0.72 | 28.85 | 40.06944444 |
| MDS27 | MDS-EB-2 | Intermediate | 0.64 | 26.86 | 41.96875 |
| MDS28 | MDS-MLD | Low | 0.39 | 37.61 | 96.43589744 |
| MDS29 | MDS-MLD | Intermediate | 0.95 | 35.71 | 37.58947368 |
| MDS31 | MDS-EB-1 | Very high | 0.45 | 25.66 | 57.02222222 |
| AML1 | AML | N/A | 1.94 | 38.99 | 20.09793814 |
| AML2 | AML | N/A | 1.06 | 35.58 | 33.56603774 |
| AML3 | AML | N/A | 0.53 | 44.88 | 84.67924528 |
| AML4 | AML | N/A | 0.91 | 25.27 | 27.76923077 |
| AML5 | AML | N/A | 1.01 | 40.87 | 40.46534653 |
| AML6 | AML | N/A | 0.48 | 26.71 | 55.64583333 |
| AML7 | AML | N/A | 2.57 | 25.96 | 10.10116732 |
| AML8 | AML | N/A | 1.71 | 25.85 | 15.11695906 |
| AML9 | AML | N/A | 2.18 | 30.57 | 14.02293578 |
| AML10 | AML | N/A | 0.66 | 27.97 | 42.37878788 |
| AML11 | AML | N/A | 0.33 | 21.99 | 66.63636364 |
| AML12 | AML | N/A | 1.57 | 30.58 | 19.47770701 |
| AML13 | AML | N/A | 0.63 | 29.21 | 46.36507937 |
| AML14 | AML | N/A | 1.49 | 32.63 | 21.89932886 |
| AML15 | AML | N/A | 0.83 | 23.83 | 28.71084337 |
| AML16 | AML | N/A | 0.73 | 31.74 | 43.47945205 |
| AML17 | AML | N/A | 0.95 | 30.78 | 32.4 |
| AML18 | AML | N/A | 2.33 | 27.48 | 11.79399142 |
| AML19 | AML | N/A | 1.10 | 23.85 | 21.68181818 |
| AML20 | AML | N/A | 0.61 | 35.49 | 58.18032787 |
| AML21 | AML | N/A | 1.04 | 25.72 | 24.73076923 |
| AML22 | AML | N/A | 0.72 | 22.69 | 31.51388889 |
| AML23 | AML | N/A | 0.47 | 18.89 | 40.19148936 |
| AML24 | AML | N/A | 1.80 | 28.36 | 15.75555556 |
| AML25 | AML | N/A | 1.04 | 29.74 | 28.59615385 |
| AML26 | AML | N/A | 1.10 | 30.83 | 28.02727273 |
| AML27 | AML | N/A | 0.57 | 26.72 | 46.87719298 |
| AML28 | AML | N/A | 0.58 | 42.96 | 74.06896552 |
| AML29 | AML | N/A | 1.00 | 34.51 | 34.51 |
| AML31 | AML | N/A | 0.67 | 43.80 | 65.37313433 |

TABLE 2

| | HTR1B | Radiolabeled | Competing | | | |
|---|---|---|---|---|---|---|
| FIG. | species | compound | compound | | Value | SEM |
| | | | | IC₅₀ (μM) | Log IC₅₀ (M) | |
| 2K | mouse | [³H]-5-HT | Kyn | 54.091 | −4.267 | 0.052 |
| | human | | | 24.444 | −4.612 | 0.071 |
| 2M | mouse | [³H]-5-HT | SB224289 | 1.2703 | −5.896 | 0.049 |
| | | [³H]-5-Kyn | | 7720.9 | −2.112 | 0.961 |
| 8J | mouse | [³H]-5-HT | 5-HT | 0.0811 | −7.091 | 0.005 |
| | human | | | 0.0597 | −7.224 | 0.076 |
| | | | | Kᵢ (μM) | Log Kᵢ (M) | |
| 8K | human | [³H]-GR125743 | Kyn | 17.306 | −4.762 | 0.051 |
| | | | 5-HT | 0.0156 | −7.808 | 0.021 |
| | | | Ergotamine | 0.0006 | −9.246 | 0.020 |
| | | | | EC₅₀ (nM) | Log EC₅₀ (M) | |
| 2L | human | CAMP Gᵢ agonist | 5-HT | 0.0298 | −10.526 | 0.038 |
| | | | Kyn | 771.68 | −6.113 | 0.206 |

TABLE 3
Off Target sites for mouse sgRNA 146 (PAM in bold):
CGCCAUGGUGAUGUACCCCA GGG (SEQ ID 1)

| | Mis | Chromo- | | | | SEQ ID |
|---|---|---|---|---|---|---|
| Off target site | matches | some | Cut site | PAM | Gene | NO. |
| AGCCATGATGATGCACCACA | 4 | chr10 | 60,341,410 | AGG | Cdh23 | 2 |
| CCCCATGGTGATGGACAGCA | 4 | chr13 | 50,226,577 | TGG | | 3 |
| CCCCATGGTGATGGACAGCA | 4 | chr13 | 50,283,597 | TGG | | 4 |
| CCCCATGGTGATGGACAGCA | 4 | chr13 | 50,579,019 | TGG | | 5 |
| CCCCAAGGTGATGTTCACCA | 4 | chr10 | 128,886,474 | AGG | Gdf11 | 6 |
| CGCCATGATTATGTCCCTCA | 4 | chr10 | 46,234,609 | TGG | RP23- | 7 |
| | | | | | 373D18.1 | |
| CACCATGGAGATCAACCCCA | 4 | chr12 | 110,661,535 | AGG | Dync1h1 | 8 |
| CACCATGGTGATGTACACAG | 4 | chr6 | 34,038,251 | TGG | Lrguk | 9 |
| CCCCATGGTGATGGATGCCA | 4 | chr14 | 21,020,749 | AGG | Vcl | 10 |
| AGCCATGGTGAGGTCCCCGA | 4 | chr11 | 100,431,148 | TGG | Nt5c3b | 11 |
| GGCCATGTGGATGAACCCCA | 4 | chr17 | 86,161,420 | TGG | | 12 |
| TGCCATGGGGATGCATCCCA | 4 | chr17 | 86,551,588 | TGG | Prkce | 13 |
| CCCCATGGTTATCTACCCAA | 4 | chr6 | 139,151,594 | AGG | | 14 |
| CGCCTTGCTGATGGTCCCCA | 4 | chr7 | 4,929,271 | CGG | Ssc5d | 15 |
| CCCCACGGTGATGAGCCCCA | 4 | chr7 | 6,709,157 | TGG | Peg3 | 16 |
| CACCATGGTGGTGTTCTCCA | 4 | chr1 | 123,785,091 | AGG | Dpp10 | 17 |
| CTCCATGGTGAAGAGCCCCA | 4 | chr3 | 57,478,764 | TGG | Wwtr1 | 18 |
| CTCCATTGAGATGTATCCCA | 4 | chr17 | 27,340,794 | GGG | | 19 |
| CTCCAAGGTGATGTCACCCA | 4 | chr6 | 120,926,177 | GGG | | 20 |
| CTCCATGATGCTGTCCCCCA | 4 | chr9 | 42,203,508 | AGG | RP24- | 21 |
| | | | | | 395P13.2 | |

TABLE 4
Off-target sites for mouse sgRNA 196 (PAM in bold):
CUGCCCACACUGAGCACGGA CGG (SEQ ID 22

| | | | | | | SEQ |
|---|---|---|---|---|---|---|
| | Mis | | | | | ID |
| Off target site | matches | Chromosome | Cut site | PAM | Gene | NO. |
| ATGCGCACACTGAGCAGGGA | 3 | chr18 | 53,410,283 | AGG | Ppic | 23 |
| CTGTCGACACTGAGCAAGGA | 3 | chr9 | 56,600,994 | TGG | | 24 |
| CTGCCCACACTGACAACCGA | 3 | chr17 | 8,903,091 | AGG | Pde10a | 25 |
| CTGCACACTCTGAGAACGGA | 3 | chr11 | 88,200,044 | AGG | | 26 |
| ATGCCCACACTGACCACGGC | 3 | chr15 | 8,702,734 | AGG | Slc1a3 | 27 |
| GAGACCACACTGAGCACGGA | 3 | chr13 | 55,670,662 | CGG | Ddx46 | 28 |
| CTGCCAACACTGAGTACGGT | 3 | chr7 | 46,358,129 | TGG | | 29 |
| CAGCCCACACTGAGCCAGGA | 3 | chr10 | 59,908,135 | GGG | | 30 |
| CTCCCCACACTGAGTACTGA | 3 | chr9 | 99,580,416 | GGG | Dbr1 | 31 |
| CTGCCCACCCAGAGCACAGA | 3 | chr4 | 79,291,332 | TGG | | 32 |
| CTGCACACTCTGAGAACGGA | 3 | KB469739.1 | 205,468 | AGG | | 33 |
| CTGCCCACACACAGCAGGGA | 3 | chr4 | 58,987,190 | AGG | | 34 |
| CTGCCCACCTTGAACACAGA | 4 | KQ030494.1 | 412,699 | AGG | | 35 |
| CTTCCCACACTGAAGAGGGA | 4 | chr16 | 10,864,916 | GGG | Rmi2 | 36 |
| CTGCACATACTGAGCATGTA | 4 | chr17 | 45,331,462 | AGG | | 37 |
| CTGCCCCCACAGGGCATGGA | 4 | chr6 | 107,900,958 | TGG | | 38 |
| CTGACCACACTCAGGATGGA | 4 | chr6 | 116,653,512 | AGG | Rassf4 | 39 |
| GTGGCCACACTGTGCACAGA | 4 | chr2 | 78,597,530 | AGG | | 40 |

TABLE 5
Off-target sites for mouse sgRNA 203 (PAM in bold):
CAGUCCGUCCGUGCUCAGUG TGG (SEQ ID 41).

| | | | | | | SEQ |
|---|---|---|---|---|---|---|
| | Mis | Chromo- | | | | ID |
| Off target site | matches | some | Cut site | PAM | Gene | NO. |
| CAGTCCTTCCTTTCTCAGTG | 3 | chr13 | 93,024,734 | AGG | | 42 |
| AAGTCCGTGTGTGCTCAGTG | 3 | chr16 | 92,658,948 | CGG | Runx1 | 43 |
| CGCCCCGTCCGTGCTCAGTG | 3 | chr13 | 55,670,655 | TGG | Ddx46 | 44 |
| CAGTCCGTCAGAGCTCTGTG | 3 | chr1 | 69,447,470 | TGG | | 45 |
| CAGTCCGTCCATCCACAGGG | 4 | chr5 | 111,839,858 | TGG | | 46 |
| CAGTCCCACAGTGCACAGTG | 4 | chr7 | 19,965,411 | TGG | | 47 |
| CTGTCAGTCCTTGATCAGTG | 4 | chrX | 67,871,707 | TGG | | 48 |
| CAGTCTTTCCTTGCTCAGAG | 4 | chr4 | 155,513,186 | TGG | Gnb1 | 49 |
| GAGTCCGTCCGTGATCACAG | 4 | chr8 | 117,341,474 | TGG | Cmip | 50 |
| CATGCAGTCCGTGCTCAGTA | 4 | chr17 | 29,776,863 | AGG | | 51 |
| AAGCCCATCTGTGCTCAGTG | 4 | chr17 | 51,966,262 | AGG | | 52 |
| CACTCCGTCCTTTGTCAGTG | 4 | chr13 | 113,684,953 | TGG | | 53 |
| CAGTCAGTCCCTGGACAGTG | 4 | chr5 | 36,203,728 | GGG | Sorcs2 | 54 |
| CAGTCAGTCTCTGGTCAGTG | 4 | chr12 | 86,777,361 | GGG | | 55 |
| CAGTGCGTCTCAGCTCAGTG | 4 | chr11 | 96,300,894 | CGG | Hoxb6 | 56 |
| CCGTCCGTCCCAGCTCAGCG | 4 | chr1 | 136,365,041 | GGG | | 57 |
| CATTCCTTCTGTGCTCAGTT | 4 | chrX | 48,841,373 | TGG | | 58 |
| CAGTCAGTCCCAGCCCAGTG | 4 | chr16 | 5,821,183 | TGG | | 59 |
| CAGTCCCTCGGCCCTCAGTG | 4 | chr13 | 8,995,828 | CGG | Gtpbp4 | 60 |

TABLE 6
Off-target sites for mouse sgRNA 610 (PAM in bold):
UAGGGAACAGCAAUAUUGCG GGG (SEQ ID 61)

| | | | | | | SEQ |
|---|---|---|---|---|---|---|
| | Mis | Chromo- | | | | ID |
| Off target site | matches | some | Cut site | PAM | Gene | NO |
| TAGGGATCAGCACTATTGGG | 3 | chr3 | 86,365,291 | AGG | Lrba | 62 |
| TAGGGAACAGCAATATTATA | 3 | chr7 | 71,213,776 | AGG | | 63 |
| TAGTGAACAGCAATTTTGTG | 3 | chr2 | 76,604,029 | GGG | | 64 |
| TAGGGAACAGCACTATTAGG | 3 | chr6 | 4,819,803 | AGG | | 65 |
| TAGGAAACAGTAATATTGCT | 3 | chr8 | 110,392,094 | GGG | Hydin | 66 |
| TAGAGAACATCAGTATTGCG | 3 | chr4 | 83,939,713 | TGG | | 67 |
| TAGGGAACAGCACTATTAGG | 3 | chr15 | 75,875,839 | AGG | | 68 |
| TAGGGAACAGGAATCTTGGG | 3 | chr7 | 63,666,183 | AGG | Otud7a | 69 |
| TAGGGAATAGCACTATTGGG | 3 | chr8 | 101,517,216 | GGG | | 70 |
| CAGGGAACAGCACTATTGCA | 3 | chr1 | 6,277,631 | AGG | | 71 |
| TAGGGAACGGCACTATTGGG | 3 | chr5 | 10,798,113 | AGG | | 72 |
| TAAGGAAAAGCAATATTGAG | 3 | chr15 | 92,388,776 | GCG | | 73 |
| TAGGGAGCGGCACTATTGGG | 4 | chr14 | 120,132,592 | AGG | | 74 |
| TAGGGAACGGCACTATTAGG | 4 | chr10 | 38,883,142 | AGG | | 75 |
| TGGGGAACAGCACTATTAAG | 4 | chr11 | 106,508,914 | AGG | Tex2 | 76 |
| TAGGGAATAGCACTATTAGG | 4 | chr11 | 118,822,144 | AGG | Rbfox3 | 77 |
| TAGGGAGTAGCACTATTGGG | 4 | chr18 | 56,714,513 | AGG | Lmnb1 | 78 |
| CAGGGAGCAGCACTATTGGG | 4 | chr18 | 66,386,447 | AGG | | 79 |
| TAGGGAATAGCACTATTAAG | 4 | chr5 | 98,747,763 | AGG | 1700007G11Rik | 80 |
| TAGGGAATGGCAGTATTGAG | 4 | chr3 | 100,784,019 | AGG | | 81 |

TABLE 7
Off-target sites for human sgRNA 126 (PAM in bold):
GUGCAAGGCGCUGUGACUUG TGG (SEQ ID 82)

| | | | | | | SEQ |
|---|---|---|---|---|---|---|
| | Mis | Chromo- | | | | ID |
| Off target site | matches | some | Cut site | PAM | Gene | NO. |
| GTGCCAGGCTCTGTGACCTG | 3 | chr22 | 34,451,537 | AGG | | 83 |
| GTGGAAGGCGCTGTGGGTTG | 3 | chr12 | 98,644,930 | AGG | IKBIP | 84 |
| GTGCCTGGCTCTGTGACTTG | 3 | chr12 | 105,025,833 | AGG | C12orf45 | 85 |
| GTGGAAGGCACAGTGACTTG | 3 | chr20 | 36,811,135 | AGG | SOGA1 | 86 |
| GTGCTAGGGGCTGTGACTCG | 3 | chr16 | 88,840,725 | AGG | GALNS | 87 |
| TGGCAAGGCGCTGTGACTTC | 3 | chr2 | 90,110,362 | TGG | | 88 |
| TTGCATGGCGCTGAGACTTG | 3 | chr2 | 183,266,542 | GGG | | 89 |
| GGGCAAGTCGCTGTTACTTG | 3 | chr8 | 43,170,985 | GGG | HGSNAT | 90 |
| GTGCCAGGCTCTGTGACGTG | 3 | chr10 | 5,553,107 | TGG | | 91 |
| GTGCAGGCCGCTGTGGCTTG | 3 | chr8 | 144,149,383 | TGG | MROH1 | 92 |
| GAGGAAGGCGCTGTGACTGG | 3 | chr8 | 144,699,816 | GGG | | 93 |
| GTGCCAAGTGCTGTGACCTG | 4 | chr12 | 106,753,537 | AGG | RFX4 | 94 |
| ATGCCAGGCGCTGTGAATTA | 4 | chr4 | 73,594,564 | GGG | RASSF6 | 95 |
| GTGCAGGGAGCAGTGGCTTG | 4 | chr3 | 187,287,424 | TGG | MASP1 | 96 |
| GTGTCAGGCTCTGTGACTTT | 4 | chr11 | 6,748,716 | AGG | GVINP2 | 97 |
| GTGCAAGGCGCTGGGGGTAG | 4 | chr11 | 15,120,280 | AGG | INSC | 98 |
| GAGCAAGCTGCTGAGACTTG | 4 | chr21 | 38,693,857 | GGG | | 99 |
| GTGGAAAGCACTGGGACTTG | 4 | chr2 | 70,733,887 | GGG | ADD2 | 100 |
| GTGCCAGGCACTGTGCCTGG | 4 | chr2 | 205,055,086 | GGG | PARD3B | 101 |
| GTGCAAGGTGCTGGGACTAT | 4 | chr5 | 169,247,739 | AGG | SLIT3 | 102 |

TABLE 8
Off-target sites for human sgRNA 170 (PAM in bold):
UUUGCCCCACACAUAUGCCA UGG (SEQ ID 103)

| | | | | | | SEQ |
|---|---|---|---|---|---|---|
| | Mis | Chromo- | | | | ID |
| Off target site | matches | some | Cut site | PAM | Gene | NO. |
| TTTACCCCACGCATATGCCT | 3 | chr15 | 85,983,712 | GGG | | 104 |
| TCTGCCCCACACACATCCCA | 3 | chr16 | 88,983,219 | GGG | | 105 |
| TTTGCCACACAGATATGCCT | 3 | chr9 | 28,802,748 | TGG | | 106 |
| TTTCCGCCACACAGATGCCA | 3 | chr3 | 11,919,420 | GGG | | 107 |
| TTTTGCCCCCACATATGCCA | 3 | chr8 | 87,559,743 | TGG | CNBD1 | 108 |
| TTTGCCACCCACATATACCA | 3 | chr11 | 105,995,944 | TGG | MSANTD4 | 109 |
| ATTGTCCCACACATAAGCCA | 3 | chr15 | 59,335,564 | GGG | MYO1E | 110 |
| CTTCCCCCACTCATATGCCA | 3 | chr5 | 10,110,115 | AGG | | 111 |
| TTTGACACACACAGATGCCA | 3 | chr2 | 18,093,369 | GGG | KCNS3 | 112 |
| TTTGCACTACACACATGCCA | 3 | chr4 | 40,501,549 | AGG | RBM47 | 113 |
| GTTGCCCCACACCAATGCTA | 4 | chr19 | 32,911,796 | TGG | CEP89 | 114 |
| TTTCCTCCATACATATACCA | 4 | chr20 | 30,212,299 | AGG | | 115 |
| TTTTCCACACACATGTACCA | 4 | chr2 | 176,551,116 | AGG | | 116 |
| TTTTCCCCACAAATATCCCT | 4 | chr2 | 177,855,724 | GGG | PDE11A | 117 |
| ATTGCCCCACAGATGTCCCA | 4 | chr15 | 91,873,370 | GGG | SLCO3A1 | 118 |
| TTTGCCCCACAGAGATGCTT | 4 | chr16 | 76,534,118 | GGG | CNTNAP4 | 119 |
| TTTGACCCACCCACATGTCA | 4 | chr16 | 82,991,612 | AGG | CDH13 | 120 |
| CTGGCCCCAGACATATGACA | 4 | chr13 | 72,535,569 | TGG | | 121 |
| TTTGCCACAGTCATAGGCCA | 4 | chr17 | 76,617,286 | TGG | | 122 |
| TTTCCCCCACCCATATCCTA | 4 | chr15 | 26,595,407 | GGG | GABRB3 | 123 |

TABLE 9
Full Sequence List

| Sequence | | |
|---|---|---|
| ID # | Sequence | Organism |
| 1 | CGCCAUGGUGAUGUACCCCA | Synthetic |
| | GGG | |
| 2 | AGCCATGATGATGCACCACA | Synthetic |
| 3 | CCCCATGGTGATGGACAGCA | Synthetic |
| 4 | CCCCATGGTGATGGACAGCA | Synthetic |
| 5 | CCCCATGGTGATGGACAGCA | Synthetic |
| 6 | CCCCAAGGTGATGTTCACCA | Synthetic |
| 7 | CGCCATGATTATGTCCCTCA | Synthetic |
| 8 | CACCATGGAGATCAACCCCA | Synthetic |
| 9 | CACCATGGTGATGTACACAG | Synthetic |
| 10 | CCCCATGGTGATGGATGCCA | Synthetic |
| 11 | AGCCATGGTGAGGTCCCCGA | Synthetic |
| 12 | GGCCATGTGGATGAACCCCA | Synthetic |
| 13 | TGCCATGGGGATGCATCCCA | Synthetic |
| 14 | CCCCATGGTTATCTACCCAA | Synthetic |
| 15 | CGCCTTGCTGATGGTCCCCA | Synthetic |
| 16 | CCCCACGGTGATGAGCCCCA | Synthetic |
| 17 | CACCATGGTGGTGTTCTCCA | Synthetic |
| 18 | CTCCATGGTGAAGAGCCCCA | Synthetic |
| 19 | CTCCATTGAGATGTATCCCA | Synthetic |
| 20 | CTCCAAGGTGATGTCACCCA | Synthetic |
| 21 | CTCCATGATGCTGTCCCCCA | Synthetic |
| 22 | CUGCCCACACUGAGCACGGA | Synthetic |
| | CGG | |
| 23 | ATGCGCACACTGAGCAGGGA | Synthetic |
| 24 | CTGTCGACACTGAGCAAGGA | Synthetic |
| 25 | CTGCCCACACTGACAACCGA | Synthetic |
| 26 | CTGCACACTCTGAGAACGGA | Synthetic |
| 27 | ATGCCCACACTGACCACGGC | Synthetic |
| 28 | GAGACCACACTGAGCACGGA | Synthetic |
| 29 | CTGCCAACACTGAGTACGGT | Synthetic |
| 30 | CAGCCCACACTGAGCCAGGA | Synthetic |
| 31 | CTCCCCACACTGAGTACTGA | Synthetic |
| 32 | CTGCCCACCCAGAGCACAGA | Synthetic |
| 33 | CTGCACACTCTGAGAACGGA | Synthetic |
| 34 | CTGCCCACACACAGCAGGGA | Synthetic |
| 35 | CTGCCCACCTTGAACACAGA | Synthetic |
| 36 | CTTCCCACACTGAAGAGGGA | Synthetic |
| 37 | CTGCACATACTGAGCATGTA | Synthetic |
| 38 | CTGCCCCCACAGGGCATGGA | Synthetic |
| 39 | CTGACCACACTCAGGATGGA | Synthetic |
| 40 | GTGGCCACACTGTGCACAGA | Synthetic |
| 41 | CAGUCCGUCCGUGCUCAGUG | Synthetic |
| | TGG | |
| 42 | CAGTCCTTCCTTTCTCAGTG | Synthetic |
| 43 | AAGTCCGTGTGTGCTCAGTG | Synthetic |
| 44 | CGCCCCGTCCGTGCTCAGTG | Synthetic |
| 45 | CAGTCCGTCAGAGCTCTGTG | Synthetic |
| 46 | CAGTCCGTCCATCCACAGGG | Synthetic |
| 47 | CAGTCCCACAGTGCACAGTG | Synthetic |
| 48 | CTGTCAGTCCTTGATCAGTG | Synthetic |
| 49 | CAGTCTTTCCTTGCTCAGAG | Synthetic |
| 50 | GAGTCCGTCCGTGATCACAG | Synthetic |
| 51 | CATGCAGTCCGTGCTCAGTA | Synthetic |
| 52 | AAGCCCATCTGTGCTCAGTG | Synthetic |
| 53 | CACTCCGTCCTTTGTCAGTG | Synthetic |
| 54 | CAGTCAGTCCCTGGACAGTG | Synthetic |
| 55 | CAGTCAGTCTCTGGTCAGTG | Synthetic |
| 56 | CAGTGCGTCTCAGCTCAGTG | Synthetic |
| 57 | CCGTCCGTCCCAGCTCAGCG | Synthetic |
| 58 | CATTCCTTCTGTGCTCAGTT | Synthetic |
| 59 | CAGTCAGTCCCAGCCCAGTG | Synthetic |
| 60 | CAGTCCCTCGGCCCTCAGTG | Synthetic |
| 61 | UAGGGAACAGCAAUAUUGCG | Synthetic |
| | GGG | |
| 62 | TAGGGATCAGCACTATTGGG | Synthetic |
| 63 | TAGGGAACAGCAATATTATA | Synthetic |
| 64 | TAGTGAACAGCAATTTTGTG | Synthetic |
| 65 | TAGGGAACAGCACTATTAGG | Synthetic |
| 66 | TAGGAAACAGTAATATTGCT | Synthetic |
| 67 | TAGAGAACATCAGTATTGCG | Synthetic |
| 68 | TAGGGAACAGCACTATTAGG | Synthetic |
| 69 | TAGGGAACAGGAATCTTGGG | Synthetic |
| 70 | TAGGGAATAGCACTATTGGG | Synthetic |
| 71 | CAGGGAACAGCACTATTGCA | Synthetic |
| 72 | TAGGGAACGGCACTATTGGG | Synthetic |
| 73 | TAAGGAAAAGCAATATTGAG | Synthetic |
| 74 | TAGGGAGCGGCACTATTGGG | Synthetic |
| 75 | TAGGGAACGGCACTATTAGG | Synthetic |
| 76 | TGGGGAACAGCACTATTAAG | Synthetic |
| 77 | TAGGGAATAGCACTATTAGG | Synthetic |
| 78 | TAGGGAGTAGCACTATTGGG | Synthetic |
| 79 | CAGGGAGCAGCACTATTGGG | Synthetic |
| 80 | TAGGGAATAGCACTATTAAG | Synthetic |
| 81 | TAGGGAATGGCAGTATTGAG | Synthetic |
| 82 | GUGCAAGGCGCUGUGACUUG | Synthetic |
| | TGG | |
| 83 | GTGCCAGGCTCTGTGACCTG | Synthetic |
| 84 | GTGGAAGGCGCTGTGGGTTG | Synthetic |
| 85 | GTGCCTGGCTCTGTGACTTG | Synthetic |
| 86 | GTGGAAGGCACAGTGACTTG | Synthetic |
| 87 | GTGCTAGGGGCTGTGACTCG | Synthetic |
| 88 | TGGCAAGGCGCTGTGACTTC | Synthetic |
| 89 | TTGCATGGCGCTGAGACTTG | Synthetic |
| 90 | GGGCAAGTCGCTGTTACTTG | Synthetic |
| 91 | GTGCCAGGCTCTGTGACGTG | Synthetic |
| 92 | GTGCAGGCCGCTGTGGCTTG | Synthetic |
| 93 | GAGGAAGGCGCTGTGACTGG | Synthetic |
| 94 | GTGCCAAGTGCTGTGACCTG | Synthetic |
| 95 | ATGCCAGGCGCTGTGAATTA | Synthetic |
| 96 | GTGCAGGGAGCAGTGGCTTG | Synthetic |
| 97 | GTGTCAGGCTCTGTGACTTT | Synthetic |
| 98 | GTGCAAGGCGCTGGGGGTAG | Synthetic |
| 99 | GAGCAAGCTGCTGAGACTTG | Synthetic |
| 100 | GTGGAAAGCACTGGGACTTG | Synthetic |
| 101 | GTGCCAGGCACTGTGCCTGG | Synthetic |
| 102 | GTGCAAGGTGCTGGGACTAT | Synthetic |
| 103 | UUUGCCCCACACAUAUGCCA | Synthetic |
| | UGG | |
| 104 | TTTACCCCACGCATATGCCT | Synthetic |
| 105 | TCTGCCCCACACACATCCCA | Synthetic |
| 106 | TTTGCCACACAGATATGCCT | Synthetic |
| 107 | TTTCCGCCACACAGATGCCA | Synthetic |
| 108 | TTTTGCCCCCACATATGCCA | Synthetic |
| 109 | TTTGCCACCCACATATACCA | Synthetic |
| 110 | ATTGTCCCACACATAAGCCA | Synthetic |
| 111 | CTTCCCCCACTCATATGCCA | Synthetic |
| 112 | TTTGACACACACAGATGCCA | Synthetic |
| 113 | TTTGCACTACACACATGCCA | Synthetic |
| 114 | GTTGCCCCACACCAATGCTA | Synthetic |
| 115 | TTTCCTCCATACATATACCA | Synthetic |
| 116 | TTTTCCACACACATGTACCA | Synthetic |
| 117 | TTTTCCCCACAAATATCCCT | Synthetic |
| 118 | ATTGCCCCACAGATGTCCCA | Synthetic |
| 119 | TTTGCCCCACAGAGATGCTT | Synthetic |
| 120 | TTTGACCCACCCACATGTCA | Synthetic |
| 121 | CTGGCCCCAGACATATGACA | Synthetic |
| 122 | TTTGCCACAGTCATAGGCCA | Synthetic |
| 123 | TTTCCCCCACCCATATCCTA | Synthetic |

What is claimed is:

1. A method of treating leukemia comprising administering a therapeutically effective amount of an inhibitor of indoleamine 2,3 dioxygenase to a mammal in need thereof.

2. The method of claim 1, wherein the mammal is a human.

3. The method of claim 2, wherein the leukemia is acute myeloid leukemia or acute lymphoid leukemia.

4. The method of claim 3, wherein the inhibitor comprises indiximod, epacadostat, BMS-986205, navoximod, PF-0684003, KHK2455 or LY3381916 or combinations thereof.

5. The method of claim 4, wherein the inhibitor comprises epacadostat.

6. The method of claim 1, wherein the inhibitor is administered orally, intravenously, intramuscularly, topically, arterially, or subcutaneously.

7. A method of inhibiting indoleamine 2,3 dioxygenase expression comprising introducing into a eukaryotic cell an engineered, non-naturally occurring Clustered Regularly Interspaced Short Palindromic Repeats (CRISPR)-CRISPR associated (Cas) (CRISPR-Cas) system comprising one or more vectors comprising, contacting a cell with a vector comprising: a) at least one nucleotide sequence encoding a CRISPR-Cas system guide RNA that hybridizes with nucleotide sequences of exons 3 or 4 encoding for indoleamine 2,3 dioxygenase, and, b) a nucleotide sequence encoding a Cas protein.

8. The method of claim 7, wherein the Cas protein is Cas9.

9. A method of treating leukemia in a subject, comprising administering an effective amount of a modulator of indoleamine 2,3 dioxygenase to a subject.

10. The method of claim 9, wherein the modulator binds to the enzyme catalytic site of indoleamine 2,3 dioxygenase.

11. The method of claim 9, wherein the modulator is a small molecule, a polynucleotide, or an antibody or antigen-binding portion thereof.

12. The method of claim 11, wherein is modulator is a nucleic acid chosen from the group consisting of a single-stranded DNA (ssDNA), a double-stranded DNA (dsDNA), a donor/template DNA, s cDNA. a DNA encoding one or more RNAs, a sgRNA, a guide RNA (gRNA), a prime editing guide RNA (pegRNA), a microRNA (miRNA) inhibitor, a miRNA mimic, a small interfering RNA (siRNA), small synthetic RNA, a synthetic RNA, an antisense oligonucleotide, a short hairpin RNA (shRNA), a double-stranded RNA (dsRNA), an antisense RNA, a ribozyme, and combinations thereof.

13. The method of claim 12, wherein the polynucleotide is a small interfering RNA (siRNA) or an antisense molecule.

14. The method of claim 13, wherein the modulator comprises a CRISPR/Cas system.

15. The method of claim 14, wherein the CRISPR-Cas system is in the form of RNA, plasmid and protein.

16. The method of claim 9, wherein the modulator is administered orally, intravenously, intramuscularly, topically, arterially, or subcutaneously.

17. A method of treating myelodysplastic syndrome comprising administering a therapeutically effective amount of an inhibitor of indoleamine 2,3 dioxygenase to a mammal in need thereof.

18. The method of claim 17, wherein the mammal is a human.

19. The method of claim 18, wherein the inhibitor comprises indiximod, epacadostat, BMS-986205, navoximod, PF-0684003, KHK2455 or LY3381916 or combinations thereof.

20. The method of claim 19, wherein the inhibitor comprises epacadostat.

21. The method of claim 17, wherein the inhibitor is administered orally, intravenously, intramuscularly, topically, arterially, or subcutaneously.

22. A method of treating leukemia comprising administering a therapeutically effective amount of an inhibitor of serum amyloid A1 (SAA1) to a mammal in need thereof.

23. The method of claim 22, wherein the mammal is a human.

24. The method of claim 23, wherein the leukemia is acute myeloid leukemia or acute lymphoid leukemia.

25. The method of claim 24, wherein the inhibitor comprises an anti-SAA1 antibody or antigen-binding portion or combinations thereof.

26. The method of claim 25, wherein the anti-SAA1 antibody is administered orally, intravenously, intramuscularly, topically, arterially, or subcutaneously.

27. A method of treating myelodysplastic syndrome comprising administering a therapeutically effective amount of an inhibitor of serum amyloid A1 (SAA1) to a mammal in need thereof.
